(19) United States
(12) Patent Application Publication
(10) Pub. No.: US 2025/0352981 A1
(43) Pub. Date: Nov. 20, 2025

(54) PHOSPHOROUS BORON-BONDED COMPOUNDS FOR CAPTURING, STORING AND/OR UTILIZING CARBON DIOXIDE AND RELATED PRODUCTS COMPOSITIONS METHODS AND SYSTEMS

(71) Applicants: California Institute of Technology, Pasadena, CA (US); North Carolina Agricultural & Technical State University, Greensboro, NC (US)

(72) Inventors: William A. Goddard, Pasadena, CA (US); Aarya Riasati, Northridge, CA (US); Charles B. Musgrave, Evanston, IL (US); Aleksandrs Prokofjevs, Greensboro, NC (US); Nathaniel Yeboah, Greensboro, NC (US)

(21) Appl. No.: 19/209,734

(22) Filed: May 15, 2025

Related U.S. Application Data
(60) Provisional application No. 63/648,281, filed on May 16, 2024.

(51) Int. Cl. B01J 20/22; B01D 53/62; B01D 53/78; B01D 53/81; B01J 20/28; B01J 20/34; C07C 1/12

(52) U.S. Cl. CPC B01J 20/223; B01D 53/62; B01D 53/78; B01D 53/81; B01J 20/22; B01J 20/28059; B01J 20/2808; B01J 20/28083; B01J 20/28085; B01J 20/345; C07C 1/12; B01D 2251/302; B01D 2257/504; B01D 2258/06

(57) ABSTRACT

Provided herein phosphorous-boron bonded compounds which are configured for capturing CO₂ with high affinity and tunability to specific CO₂ capture needs and related products, compositions, methods and systems for capturing storing and/or utilizing carbon dioxide.

### CROSS REFERENCE TO RELATED APPLICATIONS

The present application is claims priority to U.S. provisional application No. 63/648,281, entitled “Borane—Phosphine Molecules for Greenhouse Gas Capture and Sequestration,” filed on May 16, 2024 with docket number CIT 9165-P, the content of which is incorporated herein by reference in its entirety.

### STATEMENT OF GOVERNMENT INTEREST

This invention was made with government support under Grant No. DE-SC0022230 awarded by U.S. Department of Energy. The government has certain rights in the invention.

### FIELD

The present disclosure relates to carbon capture, storage and utilization technologies. In particular, the present disclosure relates to phosphorous-boron bonded compounds for capturing storing and or utilization of carbon dioxide, and related products, compositions, methods and systems.

### BACKGROUND

Capturing carbon dioxide (CO₂) is an important goal in the fight against climate change, aiming to prevent this greenhouse gas from entering the atmosphere from industrial sources and power generation. Carbon capture and storage (CCS) technologies and technologies directed to carbon capture utilization and storage (CCUS) have been developed in particular for decarbonizing sectors that are difficult to electrify, such as cement and steel production, and for achieving net-zero emissions targets.

Despite significant investments and technological advances, however technical difficulties remain in ensuring safe, permanent storage as well as the need for some CCS and CCUS approaches to employ significant amounts of renewable energy and infrastructure investment. As a result, effective CO₂ capture and recycling remain highly challenging.

### SUMMARY

Described herein are phosphorous-boron bonded compounds which are configured for capturing CO₂ with high affinity and tunability to specific CO₂ capture needs and related products, compositions, methods and systems for capturing storing and/or utilizing cycling carbon dioxide.

According to a first aspect, a phosphorous-boron bonded compound is described which is a phosphine-borane of Formula I

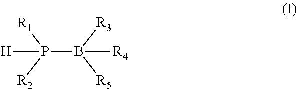

wherein
R₁, R₂ R₃, R₄ and R₅ are each independently H, F or a substituted or unsubstituted alkyl, aryl, alkoxy, aryloxy or dialkylamino group, or group of Formula II

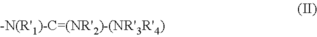

in which R′₁, R′₂, R′₃, and R′₄ are independently a substituted or unsubstituted alkyl or aryl group, and
R₁, R₂, R₃ R₄ and R₅ are configured to provide the phosphine-borane with a P—H bond having pKa 5≤pKa≤25 at 298K, at 1 atm.

According to a second aspect, a phosphorous-boron bonded compound is described which is a cyclic phosphine-borane of Formula III

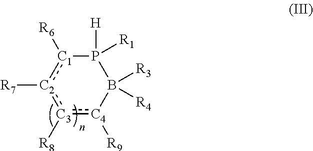

wherein
n is 0 or 1
R₁, R₃, and R₄ are each independently H, F or a substituted or unsubstituted alkyl, aryl, alkoxy, aryloxy or dialkylamino group, or group of Formula II

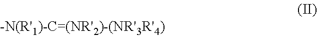

in which R′₁, R′₂, R′₃, and R′₄ are independently a substituted or unsubstituted alkyl or aryl group
R₆ is H or is joined to R₇ to form a substituted or unsubstituted and optionally heteroatom containing aliphatic or aromatic 5-membered or 6-membered ring
R₇ is H or is joined to R₆ or R₈ to form a substituted or unsubstituted and optionally heteroatom containing aliphatic or aromatic 5-membered or 6-membered ring
R₈ is H or is joined to R₇ or R₉ to form a substituted or unsubstituted and optionally heteroatom containing aliphatic or aromatic 5-membered or 6-membered ring and
R₉ is H or is joined to R₈ to form a substituted or unsubstituted and optionally heteroatom containing aliphatic or aromatic 5-membered or 6-membered ring.

According to a third aspect, a phosphorous-boron bonded compound is described which is a phosphine-borane salt of Formula IV

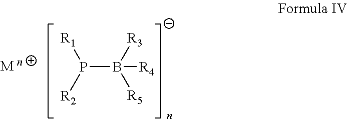

wherein
n is an integer from 1 to 3
R₁, R₂ R₃, R₄ and R₅ are each independently H, F or a substituted or unsubstituted alkyl, aryl, alkoxy, aryloxy or dialkylamino group, or group of Formula II

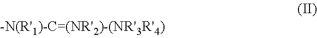

in which R′₁, R′₂, R′₃, and R′₄ are independently a substituted or unsubstituted alkyl or aryl group,
M is Li, Na, K, Rb, Cs or tetraalkylammonium when n=1; Mg, Ca, Sr, Ba when n=2 or Al when n=3, and
R₁, R₂, R₃ R₄ and R₅ are in configured such that the phosphine borane exhibits a reactivity toward CO₂ with a ΔG value <0 kcal/mol at 298K, at 1 atm.

According to a fourth aspect, a phosphorous-boron bonded compound is described which is a phosphinoborane-carbon dioxide complex of Formula V

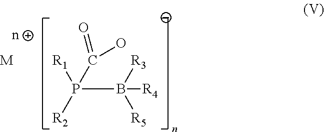

wherein
n is an integer from 1 to 3
R₁, R₂ R₃, R₄ and R₅ are each independently H, F or a substituted or unsubstituted alkyl, aryl, alkoxy, aryloxy or dialkylamino group, or group of Formula II

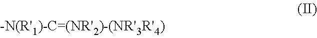

in which R′₁, R′₂, R′₃, and R′₄ are independently a substituted or unsubstituted alkyl or aryl group, and
M is Li, Na, K, Rb, Cs or tetraalkylammonium when n=1; Mg, Ca, Sr, Ba when n=2 or Al when n=3.

According to a fifth aspect, a phosphorous boron-bonded composition comprising at least one phosphorous-boron bonded compound selected from a phosphine-borane of Formula I, a cyclic phosphine-borane of Formula III and a phosphine-borane salt of Formula IV, together with a suitable vehicle.

According to a sixth aspect, a method and systems are described to make a phosphorous boron-bonded composition of the disclosure.

The method comprises mixing the at least one phosphorous boron-bonded compound with the suitable vehicle to obtain a phosphorous boron-bonded composition in which the at least one phosphorous boron-bonded compound is contained in the vehicle.

The system comprises one or more of the at least one phosphorous-boron bonded compound of the disclosure, in combination with one or more suitable vehicle, in the system of the sixth aspect at least one of the phosphorous-boron bonded compound is comprised in a form and amount configured to be mixed with the one or more suitable vehicle to provide the phosphorous boron-bonded composition of the disclosure.

According to a seventh aspect, a method and systems are described for capturing carbon dioxide from a target environment using one or more phosphorous boron-bonded compounds of the disclosure.

The method comprises: contacting the target environment with at least one phosphorous boron-bonded compound of the disclosure selected from a phosphine-borane of Formula I, a cyclic phosphine-borane of Formula III and a phosphine-borane salt of Formula IV. In the method, the contacting is performed for a time and under condition to allow binding of CO2 if present with the at least one phosphorous boron-bonded compound to form a phosphino-borane-carbon dioxide complex which is the phosphino-borane-carbon dioxide complex Formula V of the present disclosure.

The system comprises at least one phosphorous-boron bonded compound of the disclosure selected from a phosphine-borane of Formula I, a cyclic phosphine-borane of Formula III and a phosphine-borane salt of Formula IV, in combination with an equipment configured for contacting at least one phosphorous-boron bonded compound with the target environment to provide a phosphino-borane-carbon dioxide complex which is the phosphino-borane-carbon dioxide complex Formula V of the present disclosure.

According to an eighth aspect, a phosphino-borane composition is described comprising a phosphino-borane-carbon dioxide complex within a suitable vehicle, wherein the phosphinoborane-carbon dioxide complex is a complex of Formula V of the present disclosure.

According to a ninth aspect, a method and a system are described for converting carbon dioxide from a target environment to a carbon dioxide reaction product.

The method comprises: capturing the carbon dioxide from the target environment by contacting the target environment with one or more phosphorous boron-bonded compounds of the disclosure selected from a phosphine-borane of Formula I, a cyclic phosphine-borane of Formula III and a phosphine-borane salt of Formula IV, the contacting performed to provide the phosphorous boron-bonded compound of Formula V. The method further comprises reacting the compound of Formula V with a reducing agent and/or an alkylating agent to obtain the carbon dioxide reaction product.

The system comprises at least one phosphorous boron-bonded compound of the disclosure selected from a phosphine-borane of Formula I, a cyclic phosphine-borane of Formula III and a phosphine-borane salt of Formula IV, in combination with a reducing agent and/or an alkylating agent for converting a phosphinoborane-carbon dioxide complex to a carbon dioxide reaction product,

According to a tenth aspect, method and a system are described for making a carbon dioxide reaction product.

The method comprises reacting the compound of Formula V with a reducing agent and/or an alkylating agent to obtain the carbon dioxide reaction product of Formula VI

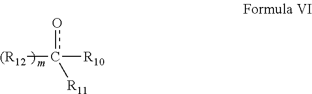

wherein
m is 0 or 1;
R₁₀, =H, OH, O-alkyl, O-aryl, CH₃, C(OH)H₂, C(O)H or C(O)OH
R₁₁ and R₁₂ are each independently H, OH, O-alkyl or O-aryl and
   wherein when m=0 and R₁₀=OH, R₁₁ is H, O-alkyl, or O-aryl

The system comprises one or more compound of Formula V with a reducing agent and/or an alkylating agent to in configuration allowing combination to obtain the carbon dioxide reaction product of Formula VI.

According to eleventh aspect a computer implemented method is described for selecting a substituent configuration of a phosphorous boron-bond compound of the disclosure, the substituent configuration having energy change(ΔGₛₑₜ) equal to or lower than a set free energy change (ΔGₛₑₜ) for carbon dioxide binding.

The method comprises modeling structures of the phosphorous boron-bond compound with varying substituents and counterions in a simulated environment, to obtain modeled structures of the phosphorous boron-bond compound in the simulated environment.

The method further comprises modeling a plurality of structures of the phosphorous boron-bond compound, the plurality of structures comprising different substituents attached to the phosphorus atom, said substituents including electron-donating groups and electron-withdrawing groups.

The method also comprises modeling structures of the phosphorous boron-bond compound in presence or absence of one or more counterion obtaining a plurality of modeled structures of the phosphorous boron-bond compound.

The method additionally comprises calculating a free energy change (ΔG) for carbon dioxide binding for each modeled structure of the plurality of modeled structures by determining the free energies of the respective lowest-energy reactant and product conformers, to obtain calculated free energy change (ΔG) of carbon dioxide binding of the modeled structures of the phosphorous boron-bond compound.

The method further comprising selecting a substituent configuration and counterions having a calculated free energy changes (ΔG) equal or to or lower than the set free energy change (ΔGₛₑₜ).

In the method the phosphorous boron-bond compound of the disclosure selected from a phosphine-borane of Formula I, a cyclic phosphine-borane of Formula III and a phosphine-borane salt of Formula IV.

According to a twelfth aspect, a device or system are described for capturing carbon dioxide from a fluid. The device or system comprise:
a) a capture inlet configured to receive the fluid;
b) a capture reaction chamber in fluid communication with the capture inlet, the capture reaction chamber containing a carbon-dioxide-absorbing material;
c) a capture outlet in fluid communication with the capture reaction chamber, configured to discharge the phosphine-borane-carbon-dioxide complex from the capture reaction chamber.

In the device or system of the twelfth aspect, the carbon-dioxide-absorbing material comprises at least one phosphorous boron-bond compound selected from a phosphine-borane of Formula I, a cyclic phosphine-borane of Formula III and a phosphine-borane salt of Formula IV,

In the device or system of the twelfth aspect, the carbon-dioxide-absorbing material is further configured to present at least one phosphorous boron-bond compound for contacting with carbon dioxide gas (CO2) from the fluid passing through the capture reaction chamber to produce a phosphine-borane-carbon-dioxide complex of Formula V; and

According to a thirteenth aspect, a device or system are described for capturing carbon dioxide from a fluid. The device or system comprise:
a) a conversion inlet configured to receive a phosphine-borane-carbon-dioxide complex of Formula V;
b) a conversion reaction chamber in fluid communication with the release inlet, the release reaction chamber being configured to convert the phosphine-borane-carbon-dioxide complex of Formula V into the CO₂ reaction product of Formula VI in presence of a reducing agent; and
c) a conversion outlet in fluid communication with the release reaction chamber, configured to discharge the CO₂ reaction product of Formula VI from the release reaction chamber.

According to a fourteenth aspect, a device or system are described for converting carbon dioxide in a fluid into a carbon dioxide reaction product. The device or system comprise:
a) a capture inlet configured to receive the fluid;
b) a capture reaction chamber in fluid communication with the capture inlet, the capture reaction chamber containing a carbon-dioxide-absorbing material comprising at least one phosphorous boron-bond compound selected from a phosphine-borane of Formula I, a cyclic phosphine-borane of Formula III and a phosphine-borane salt of Formula IV;
c) a capture outlet in fluid communication with the capture reaction chamber, configured to discharge the phosphine-borane-carbon-dioxide complex of Formula V from the capture reaction chamber into a release inlet configured to receive the phosphine-borane-carbon-dioxide complex of Formula V;
d) a conversion reaction chamber in fluid communication with the release inlet, the release reaction chamber being configured to convert the phosphine-borane-carbon-dioxide complex of Formula V into carbon dioxide reaction product of Formula VI; and
e) a conversion outlet in fluid communication with the release reaction chamber, configured to discharge the carbon dioxide reaction product of Formula VI from the release reaction chamber.

In the device and system of the fourteenth aspect, the carbon-dioxide-absorbing material is configured to present at least one phosphorous boron-bond compound for contacting with carbon dioxide gas (CO2) from the fluid passing through the capture reaction chamber to produce a phosphine-borane-carbon-dioxide complex of Formula V.

According to a fifteenth aspect, a device or system are described for scrubbing carbon dioxide out of a fluid. The device or system comprise:
a) an inlet for the fluid into the device;
b) a filter comprising at least one phosphorous boron-bond compound selected from a phosphine-borane of Formula I, a cyclic phosphine-borane of Formula III and a phosphine-borane salt of Formula IV,
   the filter configured to present at least one phosphorous boron-bond compound for contacting with carbon dioxide gas from the fluid passing through the capture reaction chamber to produce a phosphine-borane-carbon-dioxide complex of Formula V; and
c) an outlet expelling the fluid from the device after passing through the filter, the fluid have at least a portion of the phosphine-borane-carbon-dioxide complex of Formula V removed from the filter.

The phosphorous-boron bonded compounds herein described and related products, compositions, methods and systems, allow in several embodiments capturing storing and/or utilizing carbon dioxide. with high affinity (ΔG value <−5 kcal/mol at 298K, at 1 atm).

The phosphorous-boron bonded compounds herein described and related products, compositions, methods and systems, allow in several embodiments providing phosphorous-boron bonded compounds tuned to specific CO₂ capture needs.

In particular, the phosphorous-boron bonded compounds herein described and related products, compositions, methods and systems, have a configuration with easily tunable side groups, easily tunable ΔG values, and easily tunable kinetic properties from the transition state, with more sterically encumbered side groups decreasing the rate of reaction if desired and thus allowing different types of CO₂ capture.

The phosphorous-boron bonded compounds herein described and related products, compositions, methods and systems, can be used in connection with applications wherein capturing of carbon dioxide, related storage and utilization and in particular carbon dioxide recycling are desired.

Exemplary applications, comprise green technologies and environmental application for Carbon Capture, Utilization, and Storage (CCUS), such as capturing CO₂ from industrial flue gases or directly from the atmosphere, optionally followed by long-term storage (sequestration) or conversion into useful chemicals (utilization), and/or air quality improvement by removing CO₂ as a component of broader air purification or gas separation technologies.

Exemplary applications, further comprise design, prediction, synthesis, and characterization of phosphorous boron-bonded compounds with specific functional properties in material science applications as well as development of adsorbents: Creating new solid or liquid materials capable of selectively adsorbing specific gases like CO₂.

Exemplary applications, additionally comprise reagent developments for the synthesis and uses of the phosphorous boron-bonded compounds in synthetic chemistry application:

The details of one or more embodiments of the disclosure are set forth in the accompanying drawings and the description below. Other features and objects will be apparent from the description and drawings, and from the claims.

### BRIEF DESCRIPTION OF DRAWINGS

The accompanying drawings, which are incorporated into and constitute a part of this specification, illustrate one or more embodiments of the present disclosure and, together with the description of example embodiments, explain the principles and implementations of the disclosure.

FIG. 1A shows a schematic exemplary illustration of the reactivity of an exemplary Phosphido-Boranes (BoPh's) of the disclosure with CO₂.

FIG. 1B. shows a schematic exemplary illustration of the reactivity of an exemplary BoPh with electron-donating and electron-withdrawing substituents on both the boron and phosphorus moieties. The binding energy between the electron-donating and electron-withdrawing substituents is substantial, by 23 kcal/mol.

FIG. 1C shows a schematic exemplary illustration of the reactivity of an exemplary BoPh with electron-donating and electron-withdrawing substituents on both the boron and phosphorus moieties. Electron-donating based BoPhs have much lower DGs compared to the electron-withdrawing analogues. Compounds 2.1-2.3 are contain electron-withdrawing R-groups, and compounds 2.4-2.5 contain electron-donating R-groups.

FIG. 2 shows a schematic illustrations of the effect of the counterion on overall ΔG of the reaction of exemplary Phosphido-Boranes (BoPh's) with CO₂ and the effect of Li⁺ on stability.

FIG. 3 shows a schematic representation of the effect of distance of the counterions on the ΔG and the effect of Li⁺ on CO₂ binding energetics.

FIG. 4 shows a schematic representation of an exemplary reaction scheme for CO₂ capture with phosphorous-boron bonded compounds of the present disclosure.

FIG. 5 shows a schematic representation of the DFT-predicted CO₂ binding energies (kcal/mol) in THF solvent with Li counterions for exemplary BoPh's of the present disclosure Here P is purple, B is green, C is grey, H is white, and Li is blue. pKa values were tabulated in water solvent, following the equation

$$pKa = \frac{1}{2.3 RT} D ,$$

where D is the free energy change associated with dissociation of BH⁺ to B⁻ and H⁺ in water solvent. The value of H⁺ is determined to be −259.5 kcal/mol, for the solvation free energy change of a proton.

FIG. 6 shows a schematic representation of explicit solvation of the [(Ph)₂PBH₃] phosphido-borane. Explicit solvation increases overall ΔG by +2.9 kcal/mol, but it is still bound by 9.9 kcal/mol.

FIG. 7 shows a schematic representation of DFT-predicted binding and transition state energies at 300 K for the Li and Na-containing diphenylphosphido-boranes in THF solvent. The pink X⁺ indicates the counterion. The blue and black paths indicate the potential energy surfaces for Li⁺ and Na⁺, respectively.

FIG. 8 shows a schematic representation of DFT-predicted CO₂ binding energies (kcal/mol) in THE solvent with Li counterions for various BoPh's. In the illustration of FIG. 8, P is purple, B is green, C is grey, H is white, Li is dark blue, N is light blue, O is red, and F is turquoise.

FIG. 9 shows a schematic representation of DFT-predicted CO₂ binding energies (kcal/mol) in THF solvent with methyl and phenyl BoPh's with no counterion, and with Li⁺, Na⁺, and K⁺. Here P is purple, B is green, C is grey, H is white, Li is dark blue, Na is pink, K is yellow, and O is red.

FIG. 10 shows the ³¹P NMR resonance spectra of Ph₂BoPh (FIG. 10 Panel a), Li[Ph₂PBH₃] salt (FIG. 10 Panel b) and the Li[Ph₂PBH₃]—CO₂ adduct (FIG. 10 Panel c). in particular FIG. 10 shows a) ³¹P NMR of Li[Ph₂PBH₃](red spectra), (162 MHz, THF-d₈) δ 1.16-−0.79 (m)₋₁ b) ³¹P NMR of LiPh₂PBH₃ (blue spectra), (162 MHz, THF-d₈) δ −32.87 (d, J=60.7 Hz), and c) ³¹P NMR of LiPh₂PBH₃—CO₂(green spectra), (162 MHz, THF-d₈) δ 12.86.

FIG. 11 shows the ¹H NMR of FIG. 11 Panel a) Ph₂PHBH₃ (red), FIG. 11 Panel b) LiPh₂PBH₃(blue) and FIG. 11 Panel c) LiPh₂PBH₃—CO₂(green). In particular the illustration of FIG. 11 shows FIG. 11 Panel a)¹H NMR of Ph₂PHBH₃ (red spectra), (400 MHz, THF-d₈) δ 7.75-7.65 (m, 5H), 7.53-7.40 (m, 7H), 6.83 (q, J=7.1 Hz, 1H), 5.88 (q, J=7.1 Hz, 1H), 1.04 (dd, J=199.4, 94.5 Hz, 4H). FIG. 11 Panel b) ¹H NMR of LiPh₂PBH₃(blue spectra), (400 MHz, THF-d₈) δ 7.43 (ddd, J=8.0, 6.1, 1.5 Hz, 5H), 7.00 (td, J=7.2, 1.3 Hz, 5H), 6.93-6.85 (m, 2H), 0.67 (dd, J=174.9, 86.6 Hz, 5H). FIG. 11 Panel c)¹H NMR of LiPh₂PBH₃—CO₂(green spectra), (400 MHz, THF-d₈) δ 7.80 (ddt, J=9.9, 6.7, 1.6 Hz, 5H), 7.41-7.28 (m, 7H), 1.42-0.60 (m, 4H).

FIG. 12 shows ¹³C NMR of FIG. 12 Panel a) Ph₂PHBH₃ (red), FIG. 12 Panel b) LiPh₂PBH₃(blue) and FIG. 12 Panel c) LiPh₂PBH₃—CO₂(green). In particular the illustration of FIG. 12 shows FIG. 12 Panel a)¹³C NMR of Ph₂PHBH₃ (red spectra), (101 MHz, THF-d₈) δ 132.88, 132.79, 131.29, 131.27, 128.87, 128.76, 66.22, 66.00, 24.15, 23.95. FIG. 12 Panel b)¹³C NMR of LiPh₂PBH₃(blue), (101 MHz, THF-d₈) δ 135.32, 135.17, 128.21, 128.16, 125.68, 68.87, 68.73, 68.51, 26.73, 26.61, 26.53, 26.41, 26.33. FIG. 12 Panel c)¹³C NMR of LiPh₂PBH₃—CO₂(green spectra), (101 MHz, THF-d₈) δ 172.95, 172.11, 135.36, 135.28, 131.62, 131.60, 129.47, 129.37, 68.86, 68.73, 68.64, 68.51, 26.74, 26.61, 26.54, 26.41, 26.33.

FIG. 13 shows ¹H NMR spectra of Ph₂PHBH₃.

FIG. 14 shows ³¹P NMR spectra of Ph₂PHBH₃.

FIG. 15 shows ¹¹B NMR spectra of Ph₂PHBH₃.

FIG. 16 shows ¹³C NMR spectra of Ph₂PHBH₃.

FIG. 17 shows ¹H NMR Spectrum of NaPh₂PBH₃.

FIG. 18 shows ³¹P NMR Spectrum of NaPh₂PBH₃.

FIG. 19 shows ¹¹B NMR Spectrum of NaPh₂PBH₃.

FIG. 20 shows ¹³C NMR Spectrum of NaPh₂PBH₃.

FIG. 21 shows ¹H NMR Spectrum of LiPh₂PBH₃.

FIG. 22 shows ³¹P NMR Spectrum of LiPh₂PBH₃.

FIG. 23 shows a ¹¹B NMR Spectrum of LiPh₂PBH₃.

FIG. 24 shows ¹³C NMR Spectrum of LiPh₂PBH₃.

FIG. 25 shows a ¹H NMR Spectrum of KPh₂PBH₃.

FIG. 26 shows a ³¹P NMR Spectrum of KPh₂PBH₃.

FIG. 27 a ¹¹B NMR Spectrum of KPh₂PBH₃.

FIG. 28 shows ¹³C NMR Spectrum of KPh₂PBH₃.

FIG. 29 shows ¹H NMR Spectrum of NaPh₂PBH₃—CO₂ Adduct.

FIG. 30 shows ³¹P NMR Spectrum of NaPh₂PBH₃—CO₂ Adduct.

FIG. 31 shows ¹¹B NMR Spectrum of NaPh₂PBH₃—CO₂ Adduct.

FIG. 32 shows ¹³C NMR Spectrum of NaPh₂PBH₃—CO₂ Adduct.

FIG. 33 shows ¹H NMR Spectrum of LiPh₂PBH₃—CO₂ Adduct.

FIG. 34 shows ³¹P NMR Spectrum of LiPh₂PBH₃—CO₂ Adduct.

FIG. 35 shows ¹¹B NMR Spectrum of LiPh₂PBH₃—CO₂ Adduct.

FIG. 36 shows ¹³C NMR Spectrum of LiPh₂PBH₃—CO₂ Adduct.

FIG. 37 shows ¹H NMR Spectrum of KPh₂PBH₃—CO₂ Adduct.

FIG. 38 shows ³¹P NMR Spectrum of KPh₂PBH₃—CO₂ Adduct.

FIG. 39 shows ¹¹B NMR Spectrum of KPh₂PBH₃—CO₂ Adduct.

FIG. 40 shows ¹³C NMR Spectrum of KPh₂PBH₃—CO₂ Adduct.

FIG. 41 shows a ³¹P NMR of (a) NaPh₂PBH₃—CO₂ (without exposure) (b) NaPh₂PBH₃—CO₂ (1 hr exposure to atmosphere) (c) NaPh₂PBH₃—CO₂ (exposed sample stored for 3 days.

FIG. 42 shows ¹H NMR Spectrum of Et₂PHBH₃.

FIG. 43 shows ³¹P NMR Spectrum of Et₂PHBH₃.

FIG. 44 shows ¹¹B NMR Spectrum of Et₂PHBH₃.

FIG. 45 shows ¹³C NMR Spectrum of Et₂PHBH₃.

FIG. 46 shows ¹H NMR Spectrum of Li(Et₂PBH₃).

FIG. 47 shows ³¹P NMR Spectrum of Li(Et₂PBH₃).

FIG. 48 shows ¹¹B NMR Spectrum of Li(Et₂PBH₃).

FIG. 49 shows ¹³C NMR Spectrum of Li(Et₂PBH₃).

FIG. 50 shows 1H NMR Spectrum of Li(Et₂PBH₃)—CO₂ Adducts.

FIG. 51 shows ³¹P NMR Spectrum of Li(Et₂PBH₃)—CO₂ Adduct.

FIG. 52 shows ¹¹B NMR Spectrum of Li(Et₂PBH₃)—CO₂ Adduct.

FIG. 53 shows ¹¹B NMR Spectrum of Li(Et₂PBH₃)—CO₂ Adduct.

FIG. 54 shows TGA of LiETPBH₃—CO₂ Adduct.

FIG. 55 shows VT NMR of NaPh₂PBH₃ and NaPh₂PBH₃—CO₂ Adduct in equilibrium.

FIG. 56 shows ³¹P NMR of Attempted O-Alkylation of NaPh₂PBH₃—CO₂ Adduct.

FIG. 57 shows ³¹P NMR of Attempted O-Alkylation of LiEt₂PBH₃—CO₂ Adduct.

FIG. 58: shows the NMR Spectra of (a) Ph₂PHBH₃, (b) Na[Ph₂PBH₃] and (c) Na[Ph₂PBH₃]—CO₂.

FIG. 59 shows a Single Crystal Analysis of Na(Ph₂PBH₃)—CO₂ Adduct.

FIG. 60 shows an example capture reaction chamber utilizing the phosphine-borane compounds to capture carbon dioxide from a fluid.

FIG. 61 shows an example conversion reaction chamber configured to further process the phosphine-borane-carbon-dioxide complex into a carbon dioxide (or carbon) based product.

FIG. 62 shows an example system combining the capture reaction chamber of FIG. 60 with a subsequent conversion reaction chamber of FIG. 61 to further process the phosphine-borane-carbon-dioxide complex into other carbon/carbon dioxide products.

FIG. 63 shows an example filter device that uses phosphine-borane to scrub carbon dioxide from air.

### DETAILED DESCRIPTION

Described herein are phosphorous-boron bonded compounds which are configured for capturing CO₂ and related products, compositions, methods and systems for capturing storing and/or utilizing cycling carbon dioxide.

A “phosphorous-boron bonded compound,” as used herein, refers to a chemical compound comprising at least one phosphorus atom and at least one boron atom, wherein the phosphorus atom is directly bonded to the boron atom via a covalent or coordinate bond. Exemplary phosphorous-boron bonded compound organic or inorganic molecules in which the phosphorus atom is in a trivalent or pentavalent state and is complexed to boron, for example, through the formation of a P—B bond as found in phosphine-borane or phosphite-borane adducts, or in cyclic or acyclic structures containing a phosphorus-boron linkage In phosphorous-boron bonded compound in the sense of the disclosure The boron moiety may be present as a borane (e.g., BH₃) or as a substituted boron group, and the phosphorus moiety can be present as a phosphine, phosphinite, phosphonite, phosphite, or other organophosphorus derivative, optionally bearing additional substituents such as alkyl, aryl, or heteroatom-containing groups as will be understood by a skilled person

Phosphorous-boron bonded compounds of the present disclosure (herein also BoPhs) are characterized by a phosphorus center directly bonded to a borane moiety (BR′₃), with the substituents bound the phosphorus and boron being configured to tune the electro donating properties of the phosphorous atom and the electro withdrawing properties of the boron atom.

In phosphorous-boron bonded compounds of the present disclosure, the boron atom in BH₃ acts as a Lewis acid, accepting electron density from the phosphorus atom, which functions as a Lewis base and this interaction stabilizes the compound while enhancing its reactivity toward carbon dioxide.

In phosphorous-boron bonded compounds of the present disclosure substituents attached to the phosphorus atom, affect the electron density electron density and nucleophilicity of the phosphorus center and can comprise as alkyl groups (e.g., methyl, ethyl, butyl), aryl groups (e.g., phenyl, fluorophenyl) as will be understood by a skilled person upon reading of the present disclosure

In phosphorous-boron bonded compounds of the present disclosure, substituents on the borane moiety, affect the electron density of the BR′₃ are possible and could modify the Lewis acidity of the borane. Exemplary substituents on the borane moiety comprise hydrogen atoms (BH₃), though substitutions with alkyl, aryl, or other groups as will be understood by a skilled person upon reading of the present disclosure

In phosphorous-boron bonded compounds of the present disclosure, reactive forms configured to react with carbon dioxide comprise an anionic P—B complex and a counterion which enhance the native properties of the BoPh by stabilizing the anionic complex and affecting CO₂ binding. Exemplary counterions comprise lithium (Li⁺), but potentially sodium (Na⁺), potassium (K⁺), or bulky organic cations (e.g., tetraalkylammonium), or other counterions identifiable by a skilled person upon reading of the present disclosure.

In embodiments of the present disclosure, phosphorous-boron bonded compounds can be designed and configured by selection of the substituents and counterions for reactive forms to obtained tailored CO2 binding properties, suitable to various CO₂ capture storage and utilization applications, such as direct air capture or industrial flue gas treatment.

In particular, a skilled person will understand that in phosphorous-boron bonded compounds of the present disclosure, the negative charge formally reside on the phosphorus atom and the anionic character of the reactive form significantly increases the nucleophilicity of phosphorus compared to neutral phosphines (e.g., R₃P). in particular, in phosphorous-boron bonded compounds the negatively charged phosphorus is highly nucleophilic, enabling it to attack the electrophilic carbon atom in CO₂. This initiates the formation of a strong adduct, where CO₂ binds to the phosphorus center. The enhanced nucleophilicity is a key factor in the compound's ability to capture CO₂ efficiently, even at low concentrations (e.g., 410 ppm in air) as will be understood by a skilled person upon reading of the present disclosure.

A skilled person will understand that in phosphorous-boron bonded compounds the borane moiety (BH₃) has a duel role First the borane moiety stabilizes the negative charge on the phosphorus through the P—B bond, preventing unwanted side reactions and maintaining the compound's reactivity. Additionally, once CO₂ binds to the phosphorus, the boron atom can interact with the oxygen atoms of CO₂, creating a cooperative binding mechanism.

In phosphorous-boron bonded compounds of the present disclosure, the phosphorus binding to the carbon of CO₂ and boron interacting with the oxygen atoms-results in a highly stable CO₂ adduct, which is reflected in the favorable binding free energies (ΔG) reported in the related paper, such as −24.0 kcal/mol for compounds with ethyl substituents on phosphorus.

According phosphorous-boron bonded compounds of the present disclosure, are configured for performing CO₂ capture with high affinity and tunability as will be understood by a skilled person upon reading of the present disclosure In particular the high affinity:results from the combination of a nucleophilic, negatively charged phosphorus and a stabilizing borane moiety. This is especially valuable for applications like direct air capture, where strong binding is needed to extract CO₂ from dilute sources. The tunability results from the fact that while the P—B bond and anionic phosphorus are fixed features, the substituents (R) and counterion (X⁺) can be varied to fine-tune the binding strength. For example: Electron-Donating R Groups (e.g., ethyl): Increase phosphorus nucleophilicity, leading to stronger CO₂ binding, while Electron-Withdrawing R Groups (e.g., phenyl): Reduce nucleophilicity, enabling weaker, potentially reversible CO₂ binding.

In phosphorous-boron bonded compounds of the present disclosure the negatively charged phosphorus center directly bonded to a boron moiety is not only a structural characterizing features of the phosphorous-boron bonded compounds of the present disclosure but also the functional core of these compounds' CO₂ capture mechanism. the negatively charged phosphorus center directly bonded to a boron allows phosphorous-boron bonded compounds here described to outperform neutral phosphine-borane adducts (e.g., R₃P—BH₃) and other Lewis acid-base systems by forming stronger, more stable CO₂ adducts.

Accordingly phosphorous-boron bonded compounds of the disclosure a versatile class of compounds that can be configured for specific CO₂ capture needs, such as permanent sequestration or reversible capture-release cycles and additional uses identifiable by a skilled person upon reading of the present disclosure

In some embodiments herein described the phosphorous-boron bonded compound comprise phosphine-borane of Formula I

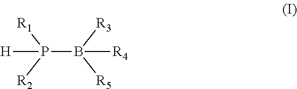

wherein
R₁, R₂ R₃, R₄ and R₅ are each independently H, F or a substituted or unsubstituted alkyl, aryl, alkoxy, aryloxy or dialkylamino group, or group of Formula II

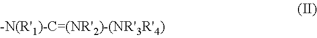

in which R′₁, R′₂, R′₃, and R′₄ are independently a substituted or unsubstituted alkyl or aryl group, and
R₁, R₂, R₃, R₄ and R₅ are configured to provide the phosphine-borane with a P—H bond having pKa 5≤pKa≤25 at 298K, at 1 atm

The term “pKa”, as defined herein, refers to the negative base-10 logarithm of the acid dissociation constant (Ka) of a compound in solution, expressed mathematically as:

$$pKa = - {log}_{10} ( Ka )$$

where Ka represents the equilibrium constant for the dissociation of an acid (HA) into its conjugate base (A⁻) and a proton (H⁺) in aqueous or solvent-based systems.

pKa can also be experimentally determined with methods identifiable by a skilled person in view of the solvent such as
potentiometric titration which encompasses titration of a solution of the acid with a strong base (or vice versa) while monitoring pH with an electrode; the pKa is derived from the inflection point of the resulting sigmoid titration curve
   or
NMR based determination which uses chemical shift imaging (CSI) to track pH-dependent changes in ¹H NMR signals. A reference compound (e.g., 2,6-dihydroxybenzoic acid) is first characterized, enabling pKa determination for other molecules in aqueous-organic solvent mixtures
   as will be understood by a skilled person.

In the context of this application, pKa quantifies the propensity of a molecule to donate or retain protons under specified conditions, thereby characterizing its acid strength. A lower pKa value indicates a stronger acid (greater proton-donating ability), while a higher pKa denotes a weaker acid as will be understood by a skilled person. The measurement or calculation of pKa, as disclosed, encompasses methods including but not limited to potentiometric titration, spectrophotometric analysis, or computational modeling, wherein the dissociation state of the compound is correlated with pH-dependent properties.

pKa values of referenced compound can be adjusted through molecular modifications (e.g., substituent effects, steric hindrance) to achieve desired performance characteristics in the final product or process as will be understood by a skilled person.

In particular, configurations with controlled pKA value can be obtained by selecting suitable electron withdrawing substituents and electron donating substituents of an initial compound, considering that electron withdrawing substituents decrease the pKa of an initial compound, whereas electron donating R-groups increase the pKa of the compound. Examples of electron-withdrawing groups are —CF3, —F, CH2OH, —Ph, and additional groups identifiable by a skilled person. Examples of electron-donating groups are —CNMe2, -t-Bu, -Et, -Me, —NH2, and additional groups identifiable by a skilled person.

In some embodiments of the phosphine-borane of Formula I, the pKa of the P—H bond is 10≤pKa≤25.

In some embodiments of the phosphine-borane of Formula I, the pKa of the P—H bond is 12≤pKa≤23.

In some embodiments of the phosphine-borane of Formula I, the pKa of the P—H bond is 5≤pKa≤17.

In some embodiments of the phosphine-borane of Formula I, the pKa of the P—H bond is 10≤pKa≤16.

In some embodiments of the phosphine-borane of Formula I, the substituted or unsubstituted alkyl or aryl are substituted or unsubstituted fluoroalkyl or fluoroaryl.

In some embodiments of the phosphine-borane of Formula I, R₁=aryl and R₂ and R₃ are both H.

In some embodiments of the phosphine-borane of Formula I, R₁=aryl and R₂, R₃, R₄ and R₅ are each H.

In some embodiments of the phosphine-borane of Formula I, R₁ and R₂=aryl and R₃, R₄ and R₅ are each H.

In some embodiments of the phosphine-borane of Formula I, R₁ and R₂=aryl and R₃, R₄ and R₅ are each F.

In some embodiments of the phosphine-borane of Formula I, R₁ and R₂=aryl and R₃, R₄ and R₅ are each C₆F₅.

In some embodiments of the phosphine-borane of Formula I, R₁=alkyl and R₂ and R₃ are both H.

In some embodiments of the phosphine-borane of Formula I, R₁ and R₂=alkyl and R₃, R₄ and R₅ are each H.

In some embodiments of the phosphine-borane of Formula I, R₁ and R₂=alkyl and R₃, R₄ and R₅ are each F.

In some embodiments of the phosphine-borane of Formula I, R₁ and R₂=alkyl and R₃, R₄ and R₅ are each C₆F₅.

In some embodiments of the phosphine-borane of Formula I, R₁=alkoxy and R₂ and R₃ are both H.

In some embodiments of the phosphine-borane of Formula I, R₁=alkoxy and R₂, R₃, R₄ and R₅ are each H.

In some embodiments of the phosphine-borane of Formula I, R₁ and R₂=alkoxy and R₃, R₄ and R₅ are each H.

In some embodiments of the phosphine-borane of Formula I, R₁ and R₂=alkoxy and R₃, R₄ and R₅ are each F.

In some embodiments of the phosphine-borane of Formula I, R₁ and R₂=alkoxy and R₃, R₄ and R₅ are each C₆F₅.

In some embodiments of the phosphine-borane of Formula I, R₁=dialkylamino and R₂ and R₃ are both H.

In some embodiments of the phosphine-borane of Formula I, R₁=dialkylamino and R₂, R₃, R₄ and R₅ are each H.

In some embodiments of the phosphine-borane of Formula I, R₁ and R₂=dialkylamino and R₃, R₄ and R₅ are each H.

In some embodiments of the phosphine-borane of Formula I, R₁ and R₂=dialkylamino and R₃, R₄ and R₅ are each F.

In some embodiments of the phosphine-borane of Formula I, R₁ and R₂=dialkylamino and R₃, R₄ and R₅ are each C₆F₅.

In some embodiments of the phosphine-borane of Formula I, R₁=fluoroalkyl and R₂ and R₃ are both H.

In some embodiments of the phosphine-borane of Formula I, R₁=fluoroalkyl and R₂, R₃, R₄ and R₅ are each H.

In some embodiments of the phosphine-borane of Formula I, R₁ and R₂=fluoroalkyl and R₃, R₄ and R₅ are each H.

In some embodiments of the phosphine-borane of Formula I, R₁ and R₂=fluoroalkyl and R₃, R₄ and R₅ are each F.

In some embodiments of the phosphine-borane of Formula I, R₁ and R₂=fluoroalkyl and R₃, R₄ and R₅ are each C₆F₅.

In some embodiments of the phosphine-borane of Formula I, n R₁=a group of Formula II and R₂ and R₃ are both H.

In some embodiments of the phosphine-borane of Formula I, R₁=a group of Formula II and R₂, R₃, R₄ and R₅ are each H.

In some embodiments of the phosphine-borane of Formula I, R₁ and R₂=a group of Formula II and R₃, R₄ and R₅ are each H.

In some embodiments of the phosphine-borane of Formula I, R₁ and R₂=a group of Formula II and R₃, R₄ and R₅ are each F.

In some embodiments of the phosphine-borane of Formula I, R₁ and R₂=a group of Formula II and R₃, R₄ and R₅ are each C₆F₅.

In some embodiments of the phosphine-borane of Formula I, R₁=F and R₂ and R₃ are both H.

In some embodiments of the phosphine-borane of Formula I, R₁=F and R₂, R₃, R₄ and R₅ are each H.

In some embodiments of the phosphine-borane of Formula I, R₁ and R₂=F and R₃, R₄ and R₅ are each H.

In some embodiments of the phosphine-borane of Formula I, each of R₁, R₂, R₃, R₄ and R₅ are F.

In some embodiments of the phosphine-borane of Formula I, R₁ and R₂=F and R₃, R₄ and R₅ are each C₆F₅.

In some embodiments of the phosphine-borane of Formula I, R₁=C₆F₅ and R₂ and R₃ are both H.

In some embodiments of the phosphine-borane of Formula I, R₁=C₆F₅ and R₂, R₃, R₄ and R₅ are each H.

In some embodiments of the phosphine-borane of Formula I, R₁ and R₂=C₆F₅ and R₃, R₄ and R₅ are each H.

In some embodiments of the phosphine-borane of Formula I, R₁ and R₂=C₆F₅ and R₃, R₄ and R₅ are each F.

In some embodiments of the phosphine-borane of Formula I, each of R₁, R₂, R₃, R₄ and R₅ are C₆F₅.

In some embodiments of the phosphine-borane of Formula I, R₁=aryl and R₂ and R₃ are both alkoxy.

In some embodiments of the phosphine-borane of Formula I, R₁=aryl and R₂, R₃, R₄ and R₅ are each alkoxy.

In some embodiments of the phosphine-borane of Formula I, R₁ and R₂=aryl and R₃, R₄ and R₅ are each alkoxy.

In some embodiments of the phosphine-borane of Formula I, R₁ and R₂=aryl and R₃ and R₄ are each alkoxy and are connected by a carbon chain of between 2 and 6 carbons.

In some embodiments of the phosphine-borane of Formula I, R₁=alkyl and R₂ and R₃ are both alkoxy.

In some embodiments of the phosphine-borane of Formula I, R₁ and R₂=alkyl and R₃, R₄ and R₅ are each alkoxy.

In some embodiments of the phosphine-borane of Formula I, R₁ and R₂=alkyl and R₃, R₄ and R₅ are each alkoxy.

In some embodiments, in which the phosphorous-boron bonded compound comprise phosphine-borane of Formula I, R₁ and R₂=alkyl and R₃ and R₄ are each alkoxy and are connected by a carbon chain of between 2 and 6 carbons.

In some embodiments of the phosphine-borane of Formula I, R₁, R₂ and R₃ are each alkoxy.

In some embodiments of the phosphine-borane of Formula I, each of R₁, R₂, R₃, R₄ and R₅ are alkoxy.

In some embodiments of the phosphine-borane of Formula I, R₁ and R₂=alkoxy and R₃ and R₄ are each alkoxy and are connected by a carbon chain of between 2 and 6 carbons.

In some embodiments of the phosphine-borane of Formula I, R₁=dialkylamino and R₂ and R₃ are both alkoxy.

In some embodiments of the phosphine-borane of Formula I, R₁=dialkylamino and R₂, R₃, R₄ and R₅ are each alkoxy.

In some embodiments of the phosphine-borane of Formula I, R₁ and R₂=dialkylamino and R₃, R₄ and R₅ are each alkoxy.

In some embodiments of the phosphine-borane of Formula I, R₁ and R₂=dialkylamino and R₃ and R₄ are each alkoxy and are connected by a carbon chain of between 2 and 6 carbons.

In some embodiments of the phosphine-borane of Formula I, R₁=a group of Formula II and R₂ and R₃ are both alkoxy.

In some embodiments of the phosphine-borane of Formula I, R₁=a group of Formula II and R₂, R₃, R₄ and R₅ are each alkoxy.

In some embodiments of the phosphine-borane of Formula I, R₁ and R₂=a group of Formula II and R₃, R₄ and R₅ are each alkoxy.

In some embodiments of the phosphine-borane of Formula I, R₁ and R₂=a group of Formula II and R₃ and R₄ are each alkoxy and are connected by a carbon chain of between 2 and 6 carbons.

In some embodiments of the phosphine-borane of Formula I, R₁=F and R₂ and R₃ are both alkoxy.

In some embodiments of the phosphine-borane of Formula I, R₁=F and R₂, R₃, R₄ and R₅ are each alkoxy.

In some embodiments of the phosphine-borane of Formula I, R₁ and R₂=F and R₃, R₄ and R₅ are each alkoxy.

In some embodiments of the phosphine-borane of Formula I, R₁ and R₂=F and R₃ and R₄ are each alkoxy and are connected by a carbon chain of between 2 and 6 carbons.

In some embodiments of the phosphine-borane of Formula I, R₁=C₆F₅ and R₂ and R₃ are both alkoxy.

In some embodiments of the phosphine-borane of Formula I, R₁=C₆F₅ and R₂, R₃, R₄ and R₅ are each alkoxy.

In some embodiments of the phosphine-borane of Formula I, R₁ and R₂=C₆F₅ and R₃, R₄ and R₅ are each alkoxy.

In some embodiments of the phosphine-borane of Formula I, R₁ and R₂=C₆F₅ and R₃ and R₄ are each alkoxy and are connected by a carbon chain of between 2 and 6 carbons.

In In some embodiments of the phosphine-borane of Formula I, R₃ is alkyl.

In some embodiments of the phosphine-borane of Formula I, both R₃ and R₄ are alkyl.

In some embodiments of the phosphine-borane of Formula I, R₃, R₄ and R₅ are each alkyl.

In some embodiments of the phosphine-borane of Formula I, R₃ is fluoroalkyl.

In some embodiments of the phosphine-borane of Formula I, both R₃ and R₄ are fluoroalkyl.

In some embodiments of the phosphine-borane of Formula I, R₃, R₄ and R₅ are each fluoroalkyl.

In some embodiments of the phosphine-borane of Formula I, R₁ is a group of Formula II and each of R′₁, R′₂, R′₃, and R′₄ are methyl groups.

In some embodiments of the phosphine-borane of Formula I, R₁ and R₂ are a group of Formula II and each of R′₁, R′₂, R′₃, and R′₄ are methyl groups.

In some embodiments of the phosphine-borane of Formula I, R₁ and R₂ are CH₃, C₂H₅ or C₄H₉ groups, R₃, R₄ and R₅ are each H and the pKa of the P—H bond is around 23.

In some embodiments of the phosphine-borane of Formula I, R₁ and R₂ are phenyl groups, R₃, R₄ and R₅ are each H and the pKa of the P—H bond is around 17.

In some embodiments of the phosphine-borane of Formula I, R₁ is a phenyl group, R₂ and R₃ are connected by a 3 carbon chain, R₄ and R₅ are each H and the pKa of the P—H bond is around 12.

In some embodiments of the phosphine-borane of Formula I, R₁ is a phenyl group, R₂ and R₃ are connected by a 4 carbon chain, R₄ and R₅ are each H and the pKa of the P—H bond is around 16.

In some embodiments of the phosphine-borane of Formula I, the compound further comprises part of the repeat unit of a polymer, where the polymer chain is connected through any one of R₁ to R₆.

In some embodiments of the phosphine-borane of Formula I, the compound further comprises part of the repeat unit of a polymer, where the polymer chain is connected through repeated B—P bonds as depicted by Formula Ta

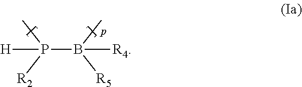

where R₁ and R₃ are B and P respectively; p is the number of P—B repeat units in the polymer.

In some embodiments of the phosphine-borane of Formula I, R₁ is B, such that the structure of Formula I comprises a bis-phosphine-borane.

In some embodiments herein described the phosphorous-boron bonded compound comprise a cyclic phosphine-borane of Formula III

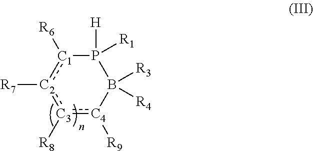

wherein
n is 0 or 1
R₁, R₃, and R₄ are each independently H, F or a substituted or unsubstituted alkyl, aryl, alkoxy, aryloxy or dialkylamino group, or group of Formula II

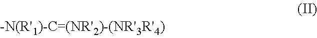

in which R′₁, R′₂, R′₃, and R′₄ are independently a substituted or unsubstituted alkyl or aryl group
R₆ is H or is joined to R₇ to form a substituted or unsubstituted and optionally heteroatom containing aliphatic or aromatic 5 membered or 6-membered ring
R₇ is H or is joined to R₆ or R₈ to form a substituted or unsubstituted and optionally heteroatom containing aliphatic or aromatic 5 membered or 6-membered ring
R₈ is H or is joined to R₇ or R₉ to form a substituted or unsubstituted and optionally heteroatom containing aliphatic or aromatic 5 membered or 6-membered ring and
R₉ is H or is joined to R₈ to form a substituted or unsubstituted and optionally heteroatom containing aliphatic or aromatic 5 membered or 6-membered ring

A bis-phosphineborane, with two anionic borane units bound to a central phosphorus, a boroxine type structure, where, instead of oxygens, there are phosphorus type moieties.

In some embodiments of the cyclic phosphine-borane of Formula III, the substituted or unsubstituted alkyl or aryl are substituted or unsubstituted fluoroalkyl or fluoroaryl.

In some embodiments of the cyclic phosphine-borane of Formula III, R₁=aryl and R₃ and R₄ are both H.

In some embodiments of the cyclic phosphine-borane of Formula III, R₁=aryl and R₃ and R₄ are both F.

In some embodiments of the cyclic phosphine-borane of Formula III, R₁=aryl and R₃ and R₄ are both C₆F₅.

In some embodiments of the cyclic phosphine-borane of Formula III, R₁=alkyl and R₃ and R₄ are both H.

In some embodiments of the cyclic phosphine-borane of Formula III, R₁=alkyl and R₃ and R₄ are both F.

In some embodiments of the cyclic phosphine-borane of Formula III, R₁=alkyl and R₃ and R₄ are both C₆F₅.

In some embodiments of the cyclic phosphine-borane of Formula III, R₁=alkoxy and R₃ and R₄ are both H.

In some embodiments of the cyclic phosphine-borane of Formula III, R₁=alkoxy and R₃ and R₄ are both F.

In some embodiments of the cyclic phosphine-borane of Formula III, R₁=alkoxy and R₃ and R₄ are both C₆F₅.

In some embodiments of the cyclic phosphine-borane of Formula III, R₁=dialkylamino and R₃ and R₄ are both H.

In some embodiments of the cyclic phosphine-borane of Formula III, R₁=dialkylamino and R₃ and R₄ are both F.

In some embodiments of the cyclic phosphine-borane of Formula III, R₁=dialkylamino and R₃ and R₄ are both C₆F₅.

In some embodiments of the cyclic phosphine-borane of Formula III, R₁=fluoroalkyl and R₃ and R₄ are both H.

In some embodiments of the cyclic phosphine-borane of Formula III, R₁=fluoroalkyl and R₃ and R₄ are both F.

In some embodiments of the cyclic phosphine-borane of Formula III, R₁=fluoroalkyl and R₃ and R₄ are both C₆F₅.

In some embodiments of the cyclic phosphine-borane of Formula III, R₁=a group of Formula II and R₃ and R₄ are both H.

In some embodiments of the cyclic phosphine-borane of Formula III, R₁=a group of Formula II and R₃ and R₄ are both F.

In some embodiments of the cyclic phosphine-borane of Formula III, R₁=a group of Formula II and R₃ and R₄ are both C₆F₅.

In some embodiments of the cyclic phosphine-borane of Formula III, R₁=F and R₃ and R₄ are both H.

In some embodiments of the cyclic phosphine-borane of Formula III, R₁, R₃ and R₄ are each F.

In some embodiments of the cyclic phosphine-borane of Formula III, R₁=F and R₃ and R₄ are both C₆F₅.

In some embodiments of the cyclic phosphine-borane of Formula III, R₁=C₆F₅ and R₃ and R₄ are both H.

In some embodiments of the cyclic phosphine-borane of Formula III, R₁=C₆F₅ and R₃ and R₄ are both F.

In some embodiments of the cyclic phosphine-borane of Formula III, R₁, R₃ and R₄ are each C₆F₅.

In some embodiments of the cyclic phosphine-borane of Formula III, R₁=aryl and R₃ and R₄ are both alkoxy.

In some embodiments of the cyclic phosphine-borane of Formula III, R₁=aryl and R₃ and R₄ are each alkoxy and are connected by a carbon chain of between 2 and 6 carbons.

In some embodiments of the cyclic phosphine-borane of Formula III, R₁=alkyl and R₃ and R₄ are both alkoxy.

In some embodiments of the cyclic phosphine-borane of Formula III, R₁=alkyl and R₃ and R₄ are each alkoxy and are connected by a carbon chain of between 2 and 6 carbons.

In some embodiments of the cyclic phosphine-borane of Formula III, R₁, R₃ and R₄ are each alkoxy.

In some embodiments of the cyclic phosphine-borane of Formula III, R₁=alkoxy and R₃ and R₄ are each alkoxy and are connected by a carbon chain of between 2 and 6 carbons.

In some embodiments of the cyclic phosphine-borane of Formula III, R₁=dialkylamino and R₃ and R₄ are both alkoxy.

In some embodiments of the cyclic phosphine-borane of Formula III, R₁=dialkylamino and R₃ and R₄ are each alkoxy and are connected by a carbon chain of between 2 and 6 carbons.

In some embodiments of the cyclic phosphine-borane of Formula III, R₁=a group of Formula II and R₃ and R₄ are both alkoxy.

In some embodiments of the cyclic phosphine-borane of Formula III, R₁=a group of Formula II and R₃ and R₄ are each alkoxy and are connected by a carbon chain of between 2 and 6 carbons.

In some embodiments of the cyclic phosphine-borane of Formula III, R₁=F and R₃ and R₄ are both alkoxy.

In some embodiments of the cyclic phosphine-borane of Formula III, R₁=F and R₃ and R₄ are each alkoxy and are connected by a carbon chain of between 2 and 6 carbons.

In some embodiments of the cyclic phosphine-borane of Formula III, R₁=C₆F₅ and R₃ and R₄ are both alkoxy.

In some embodiments of the cyclic phosphine-borane of Formula III, R₁=C₆F₅ and R₃ and R₄ are each alkoxy and are connected by a carbon chain of between 2 and 6 carbons.

In some embodiments of the cyclic phosphine-borane of Formula III, R₃ is alkyl.

In some embodiments of the cyclic phosphine-borane of Formula III, both R₃ and R₄ are alkyl.

In some embodiments of the cyclic phosphine-borane of Formula III, R₃ is fluoroalkyl.

In some embodiments of the cyclic phosphine-borane of Formula III, both R₃ and R₄ are fluoroalkyl.

In some embodiments of the cyclic phosphine-borane of Formula III, R₁ is a group of Formula II and each of R′₁, R′₂, R′₃, and R′₄ are methyl groups.

In some embodiments of the cyclic phosphine-borane of Formula III, n=0 and R₆ and R₇ comprise part of a benzene ring.

In some embodiments of the cyclic phosphine-borane of Formula III, n=0 and R₆ and R₇ comprise part of a naphthalene, anthracene, tetracene or pentacene ring.

In some embodiments of the cyclic phosphine-borane of Formula III, n=0 and R₆ and R₇ comprise part of a cyclopentane ring.

In some embodiments of the cyclic phosphine-borane of Formula III, n=0 and R₆ and R₇ comprise part of a cyclohexane ring.

In some embodiments of the cyclic phosphine-borane of Formula III, n=0 and R₆ and R₇ comprise part of a decalin ring.

In some embodiments of the cyclic phosphine-borane of Formula III, n=0 and R₆ and R₇ comprise part of a pyridine ring.

In some embodiments of the cyclic phosphine-borane of Formula III, n=0 and R₆ and R₇ comprise part of a thiophene ring.

In some embodiments of the cyclic phosphine-borane of Formula III, n=0 and R₆ and R₇ comprise part of a furan ring.

In some embodiments of the cyclic phosphine-borane of Formula III, n=0 and R₆ and R₇ comprise part of a piperidine ring.

In some embodiments of the cyclic phosphine-borane of Formula III, n=0 and R₆ and R₇ comprise part of a pyrrole ring.

In some embodiments of the cyclic phosphine-borane of Formula III, n=0 and R₆ and R₇ comprise part of a pyrrolidine ring.

In some embodiments of the cyclic phosphine-borane of Formula III, n=0 and R₆ and R₇ comprise part of a morpholine ring.

In some embodiments of the cyclic phosphine-borane of Formula III, n=1 and R₆ and R₇ comprise part of a benzene ring.

In some embodiments of the cyclic phosphine-borane of Formula III, n=1 and R₆ and R₇ comprise part of a naphthalene, anthracene, tetracene or pentacene ring.

In some embodiments of the cyclic phosphine-borane of Formula III, n=1 and R₆ and R₇ comprise part of a cyclopentane ring.

In some embodiments of the cyclic phosphine-borane of Formula III, n=1 and R₆ and R₇ comprise part of a cyclohexane ring.

In some embodiments of the cyclic phosphine-borane of Formula III, n=1 and R₆ and R₇ comprise part of a decalin ring.

In some embodiments of the cyclic phosphine-borane of Formula III, n=1 and R₆ and R₇ comprise part of a pyridine ring.

In some embodiments of the cyclic phosphine-borane of Formula III, n=1 and R₆ and R₇ comprise part of a thiophene ring.

In some embodiments of the cyclic phosphine-borane of Formula III, n=1 and R₆ and R₇ comprise part of a furan ring.

In some embodiments of the cyclic phosphine-borane of Formula III, n=1 and R₆ and R₇ comprise part of a piperidine ring.

In some embodiments of the cyclic phosphine-borane of Formula III, n=1 and R₆ and R₇ comprise part of a pyrrole ring.

In some embodiments of the cyclic phosphine-borane of Formula III, n=1 and R₆ and R₇ comprise part of a pyrrolidine ring.

In some embodiments of the cyclic phosphine-borane of Formula III, n=1 and R₆ and R₇ comprise part of a morpholine ring.

In some embodiments of the cyclic phosphine-borane of Formula III, n=0 and R₇ and R₉ comprise part of a benzene ring.

In some embodiments of the cyclic phosphine-borane of Formula III, n=0 and R₇ and R₉ comprise part of a naphthalene, anthracene, tetracene or pentacene ring.

In some embodiments of the cyclic phosphine-borane of Formula III, n=0 and R₇ and R₉ comprise part of a cyclopentane ring.

In some embodiments of the cyclic phosphine-borane of Formula III, n=0 and R₇ and R₉ comprise part of a cyclohexane ring.

In some embodiments of the cyclic phosphine-borane of Formula III, n=0 and R₇ and R₉ comprise part of a decalin ring.

In some embodiments of the cyclic phosphine-borane of Formula III, n=0 and R₇ and R₉ comprise part of a pyridine ring.

In some embodiments of the cyclic phosphine-borane of Formula III, n=0 and R₇ and R₉ comprise part of a thiophene ring.

In some embodiments of the cyclic phosphine-borane of Formula III, n=0 and R₇ and R₉ comprise part of a furan ring.

In some embodiments of the cyclic phosphine-borane of Formula III, n=0 and R₇ and R₉ comprise part of a piperidine ring.

In some embodiments of the cyclic phosphine-borane of Formula III, n=0 and R₇ and R₉ comprise part of a pyrrole ring.

In some embodiments of the cyclic phosphine-borane of Formula III, n=0 and R₇ and R₉ comprise part of a pyrrolidine ring.

In some embodiments of the cyclic phosphine-borane of Formula III, n=0 and R₇ and R₉ comprise part of a morpholine ring.

In some embodiments of the cyclic phosphine-borane of Formula III, wherein n=1 and R₇ and R₈ comprise part of a benzene ring.

In some embodiments of the cyclic phosphine-borane of Formula III, n=1 and R₇ and R₈ comprise part of a naphthalene, anthracene, tetracene or pentacene ring.

In some embodiments of the cyclic phosphine-borane of Formula III, n=1 and R₇ and R₈ comprise part of a cyclopentane ring.

In some embodiments of the cyclic phosphine-borane of Formula III, n=1 and R₇ and R₈ comprise part of a cyclohexane ring.

In some embodiments of the cyclic phosphine-borane of Formula III, n=1 and R₇ and R₈ comprise part of a decalin ring.

In some embodiments of the cyclic phosphine-borane of Formula III, n=1 and R₇ and R₈ comprise part of a pyridine ring.

In some embodiments of the cyclic phosphine-borane of Formula III, n=1 and R₇ and R₈ comprise part of a thiophene ring.

In some embodiments of the cyclic phosphine-borane of Formula III, n=1 and R₇ and R₈ comprise part of a furan ring.

In some embodiments of the cyclic phosphine-borane of Formula III, n=1 and R₇ and R₈ comprise part of a piperidine ring.

In some embodiments of the cyclic phosphine-borane of Formula III, n=1 and R₇ and R₈ comprise part of a pyrrole ring.

In some embodiments of the cyclic phosphine-borane of Formula III, n=1 and R₇ and R₈ comprise part of a pyrrolidine ring.

In some embodiments of the cyclic phosphine-borane of Formula III, n=1 and R₇ and R₈ comprise part of a morpholine ring.

In some embodiments of the cyclic phosphine-borane of Formula III, n=1 and R₈ and R₉ comprise part of a benzene ring.

In some embodiments of the cyclic phosphine-borane of Formula III, n=1 and R₈ and R₉ comprise part of a naphthalene, anthracene, tetracene or pentacene ring.

In some embodiments of the cyclic phosphine-borane of Formula III, n=1 and R₈ and R₉ comprise part of a cyclopentane ring.

In some embodiments of the cyclic phosphine-borane of Formula III, n=1 and R₈ and R₉ comprise part of a cyclohexane ring.

In some embodiments of the cyclic phosphine-borane of Formula III, n=1 and R₈ and R₉ comprise part of a decalin ring.

In some embodiments of the cyclic phosphine-borane of Formula III, n=1 and R₈ and R₉ comprise part of a pyridine ring.

In some embodiments of the cyclic phosphine-borane of Formula III, n=1 and R₈ and R₉ comprise part of a thiophene ring.

In some embodiments of the cyclic phosphine-borane of Formula III, n=1 and R₈ and R₉ comprise part of a furan ring.

In some embodiments of the cyclic phosphine-borane of Formula III, n=1 and R₈ and R₉ comprise part of a piperidine ring.

In some embodiments of the cyclic phosphine-borane of Formula III, n=1 and R₈ and R₉ comprise part of a pyrrole ring.

In some embodiments of the cyclic phosphine-borane of Formula III, n=1 and R₈ and R₉ comprise part of a pyrrolidine ring.

In some embodiments of the cyclic phosphine-borane of Formula III, n=1 and R₈ and R₉ comprise part of a morpholine ring.

In some embodiments of the cyclic phosphine-borane of Formula III, R₁ and R₆ are joined by a carbon chain of between 2 and 6 atoms

In some embodiments of the cyclic phosphine-borane of Formula III, R₃ and R₉ are joined by a carbon chain of between 2 and 6 atoms

In some embodiments of the cyclic phosphine-borane of Formula III, R₁ and R₃ are joined by a carbon chain of between 2 and 6 atoms

In some embodiments of the cyclic phosphine-borane of Formula III, the compound further comprises part of the repeat unit of a polymer, where the polymer chain is connected through any one of R₁, R₃ or R₄. In particular in those embodiments R1, R3 or R4 form part of the connection to a polymer chain, with the structure of Formula III a side chain of this polymer e.g. [CH2-CH(CxR1-P—B(cycle))]n. That is, the structure of FIII is connected to the polymer via P (R1) or B (R3, R4) atoms as shown in the following Formula III(a) and Formula III(b)

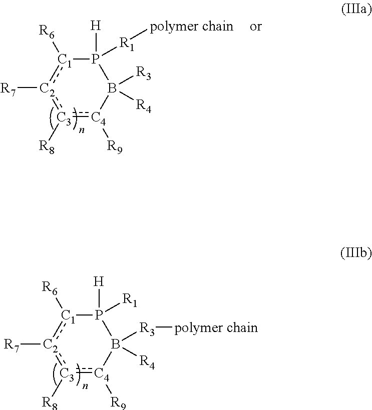

In some embodiments of the cyclic phosphine-borane of Formula III, the compound further comprises part of the repeat unit of a polymer, where the polymer chain is connected through any one of R₆ to R₉. In particular in those embodiments R6 to R9 form part of the connection to a polymer chain, with the structure of Formula III a side chain of this polymer e.g. [CH2-CH(CₓR₆—C—P—B(cycle))]n. That is, the structure of FIII is connected to the polymer via atoms that are not P or B (R1) or B (R3, R4) atoms

An exemplary configuration is depicted in the Formula (IIIc) below

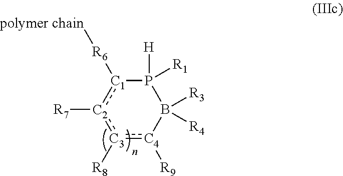

In some embodiments of the cyclic phosphine-borane of Formula III, the compound further comprises part of the repeat unit of a polymer, where the polymer chain is connected through repeated B—P bonds. as depicted by in Formula (IIId) below

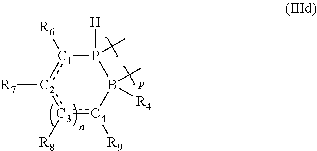

where R₁ and R₃ are B and P respectively; p is the number of P—B repeat units in the polymer.

In some embodiments of the cyclic phosphine-borane of Formula III, R₁ is B, such that the structure of Formula III comprises a bis-phosphine-borane.

In some embodiments herein described the phosphorous-boron bonded compound comprise a phosphine-borane salt of Formula IV

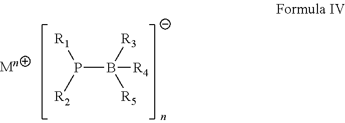

wherein
n is an integer from 1 to 3
R₁, R₂, R₃, R₄ and R₅ are each independently H, F or a substituted or unsubstituted alkyl, aryl, alkoxy, aryloxy or dialkylamino group, or group of Formula II

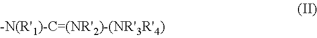

in which R′₁, R′₂, R′₃, and R′₄ are independently a substituted or unsubstituted alkyl or aryl group,
M is Li, Na, K, Rb, Cs or tetraalkylammonium when n=1; Mg, Ca, Sr, Ba when n=2 or Al when n=3, and
R₁, R₂, R₃, R₄ and R₅ are in configured such that the phosphine borane exhibits a reactivity toward CO₂ with a ΔG value <0 kcal/mol at 298K, at 1 atm.

ΔG values can be measured for example by quantifying with 1H or 31P NMR a reactant/product ratios at equilibrium and calculating Keq and derive ΔG via:

$$Δ G = - RT ln {K}_{eq} \qquad ( 1 )$$

and with other methods identifiable by a skilled person.

In embodiments of phosphorous-boron bonded compound of Formula IV, ΔG value can be modified in view of the configuration of the substituents and more notably by the selection of the counterion which can modify the overall ΔG from extremely favorable with the reaction being thus irreversible, to less favorable, and thus reversible, to almost completely irreversible as will be understood by a skilled person upon reading of the present disclosure.

In particular, in embodiments of phosphorous-boron bonded compound of Formula IV, in the indicated range of ΔG value the compounds exhibit reactivity towards SO₂ and NOₓ, but prefer CO₂. Therefore in embodiments of phosphorous-boron bonded compound of Formula IV, the ΔG value, is most favorable for CO₂ capture as a result of its abundance in the atmosphere and overall ΔG value. Within the indicated range of ΔG value compounds with ΔG values that are between 0 and −5 (0>ΔG≥−5) are configured for reversible CO₂ capture, and phosphorous-boron bonded compound with ΔG values <−5 are designed for irreversible CO₂ capture.

In some preferred embodiments of phosphorous-boron bonded compound of Formula IV, M can be or comprise Lithium (Li⁺) as the Primary Counterion. Lithium (Li⁺) has a small ionic radius (0.76 Å) and a high charge density, making it strongly polarizing and capable of forming tight ion pairs with the [R₂P—BR′₃] anion. This increases the stability of the product complex as a result of tight ion-pair formation between the CO₂—BH₃⁻ moiety and the Li⁺, which forms a binding pocket. This binding pocket enhances the stability of the product complex by over +17 kcal in the BoPh case with R-groups as phenyl on the phosphorus.

In the preferred embodiments of phosphorous-boron bonded compound of Formula IV, where M is Li⁺ the tight ion pairing with Li⁺ enhances the compound's stability by reducing the anion's reactivity and susceptibility to side reactions. This strong electrostatic interaction helps maintain the structural integrity of the compound over time. The distance has between the X⁺ and the binding pocket has a drastic effect on ΔG of CO₂ binding. As the distance is perturbed from the ideal 1.95 Å between X⁺ and BH₃⁻, and 1.81 Å between X⁺ and CO₂, ΔG increases from −12.8 kcal/mol to −6.9 kcal/mol as the distance between the X⁺, (which, in this case, Li⁺) is adjusted by 0.35 Å

In preferred embodiments of phosphorous-boron bonded compound of Formula IV, where M is Li⁺ Li⁺ significantly improves the energetics of CO₂ binding. In particular, this interaction: i) reduces the energy of the CO₂-bound state, leading to a more favorable binding free energy (e.g., ΔG=−24.0 kcal/mol for ethyl-substituted compounds), and ii) Bends the O—C—O angle from 1800 to approximately 130°, enhancing orbital overlap and strengthening the P—CO₂ bond.

Accordingly in the preferred embodiments of phosphorous-boron bonded compound of Formula IV, where M is Li⁺. The strong CO₂-binding affinity enabled by Li⁺ are preferred for applications like direct air capture or permanent CO₂ sequestration, where maximizing capture strength is essential. These effects are not as present in systems with larger cations, but are still present.

In some embodiments of phosphorous-boron bonded compound of Formula IV, M is Na⁺. In those embodiments, sodium (Na⁺) has a larger ionic radius (1.02 Å) than Li⁺, resulting in a lower charge density and weaker ion pairing with the anion.

In embodiments of phosphorous-boron bonded compound of Formula IV, where M is Na⁺ the looser ion pairing with Na⁺ provides less stabilization compared to Li⁺, potentially increasing the compound's reactivity but reducing its long-term stability.

In embodiments of phosphorous-boron bonded compound of Formula IV, where M is Na⁺, Na⁺ is less effective at coordinating with the oxygen atoms of bound CO₂ due to its larger size and weaker electrostatic influence. This leads to a less favorable binding energy (e.g., ΔG≈−20.0 kcal/mol), weaker than that observed with Li⁺.

Accordingly in embodiments of phosphorous-boron bonded compound of Formula IV, where M is Na⁺, the phosphorous-boron bonded compound of Formula IV are expected to be suitable for applications where slightly weaker CO₂ binding is acceptable, such as systems designed for easier regeneration of the capture material.

In some embodiments of phosphorous-boron bonded compound of Formula IV, M is K⁺. In embodiments of phosphorous-boron bonded compound of Formula IV, where M is K⁺, Potassium (K⁺) has an even larger ionic radius (1.38 Å), further reducing its charge density and resulting in very weak ion pairing.

In embodiments of phosphorous-boron bonded compound of Formula IV, where M is K⁺. the minimal stabilization provided by K⁺ can make the compound more prone to decomposition or unwanted reactions, compromising its stability.

In embodiments of phosphorous-boron bonded compound of Formula IV, where M is K⁺, the weak interaction between K⁺ and the anion or CO₂ leads to significantly reduced binding strength. This makes CO₂ capture less energetically favorable compared to Li⁺ or Na⁺.

Accordingly in embodiments of phosphorous-boron bonded compound of Formula IV, where M is K⁺, phosphorous-boron bonded compound are expected to be advantageous in reversible CO₂ capture systems where weak binding facilitates rapid release of CO₂ as will be understood by a skilled person upon reading of the present disclosure.

In some embodiments of phosphorous-boron bonded compound of Formula IV, M is bulky organic cations (e.g., Tetraalkylammonium [NR₄]⁺. In those embodiments the cations are large, with delocalized charge, leading to very weak ion pairing with the [R₂P-BR′₃]⁻ anion.

In embodiments of phosphorous-boron bonded compound of Formula IV, wherein M is bulky organic cations the weak ion pairing leaves the anion more “naked”, increasing its reactivity. While this may enhance the kinetics of CO₂ capture, it can reduce the compound's overall stability.

In embodiments of phosphorous-boron bonded compound of Formula IV, wherein M is bulky organic cations, the bulky organic cations do not effectively coordinate with the oxygen atoms of bound CO₂, resulting in weaker binding affinities compared to Li⁺, Na⁺, or K⁺.

Accordingly, in embodiments of phosphorous-boron bonded compound of Formula IV, wherein M is bulky organic cations these counterions are useful in liquid-phase CO₂ capture systems where solubility and fast reaction rates are prioritized over maximizing binding strength as will be understood by a skilled person upon reading of the present disclosure

In embodiments, of phosphorous-boron bonded compound of Formula IV, selection of the counterion M can be performed to affect the CO₂-Binding Affinity of the phosphorous-boron bonded compound, In particular, with respect to the exemplary Li⁺, Na⁺, K⁺ and bulky organic cations the CO₂-Binding Affinity is expected to be Li⁺>Na⁺>K⁺>Bulky Organic Cations. Li⁺ offers the strongest binding due to its ability to tightly pair with the anion and stabilize the CO₂ adduct. Binding strength decreases with larger counterions as coordination weakens.

In embodiments, of phosphorous-boron bonded compound of Formula IV, selection of the counterion M can be performed to affect the Ion pairing Strength: In particular, with respect to the exemplary Li⁺, Na⁺, K⁺ and bulky organic cations the ranking is Li⁺ (tight)>Na⁺>K⁺>bulky organic cations, in those embodiments, (loose) that tight ion pairing with Li⁺ maximizes stability and CO₂-binding strength, while looser pairing with larger cations increases reactivity but reduces control over the anion.

In embodiments, of phosphorous-boron bonded compound of Formula IV, selection of the counterion M can be performed to affect solubility and application: in this respect and with respect to the exemplary Li⁺, Na⁺, K⁺ and bulky organic cations Li⁺ salts are highly soluble in polar solvents (e.g., THF), making them versatile for many systems as will be understood by a skilled person upon reading of the present disclosure

Considerations which can be used to identify proper counterion M include the ability of smaller, more positively charged counterions (e.g. Li⁺, Na⁺, K). stabilize the product state, increasing the value of ΔG. Larger, more diffuse counterions are not able to as favorably stabilize the product, leading to decreases in ΔG, thus making the phosphorous-boron bonded compound of Formula IV more negative.

In some embodiments of the phosphine-borane salt of Formula IV, the substituted or unsubstituted alkyl or aryl are substituted or unsubstituted fluoroalkyl or fluoroaryl.

In some embodiments of the phosphine-borane salt of Formula IV, R₁=aryl and R₂ and R₃ are both H.

In some embodiments of the phosphine-borane salt of Formula IV, R₁=aryl and R₂, R₃, R₄ and R₅ are each H.

In some embodiments of the phosphine-borane salt of Formula IV, R₁ and R₂=aryl and R₃, R₄ and R₅ are each H.

In some embodiments of the phosphine-borane salt of Formula IV, R₁ and R₂=aryl and R₃, R₄ and R₅ are each F.

In some embodiments of the phosphine-borane salt of Formula IV, R₁ and R₂=aryl and R₃, R₄ and R₅ are each C₆F₅.

In some embodiments of the phosphine-borane salt of Formula IV, R₁=alkyl and R₂ and R₃ are both H.

In some embodiments of the phosphine-borane salt of Formula IV, R₁ and R₂=alkyl and R₃, R₄ and R₅ are each H.

In some embodiments of the phosphine-borane salt of Formula IV, R₁ and R₂=alkyl and R₃, R₄ and R₅ are each F.

In some embodiments of the phosphine-borane salt of Formula IV, R₁ and R₂=alkyl and R₃, R₄ and R₅ are each C₆F₅.

In some embodiments of the phosphine-borane salt of Formula IV, R₁=alkoxy and R₂ and R₃ are both H.

In some embodiments of the phosphine-borane salt of Formula IV, R₁=alkoxy and R₂, R₃, R₄ and R₅ are each H.

In some embodiments of the phosphine-borane salt of Formula IV, R₁ and R₂=alkoxy and R₃, R₄ and R₅ are each H.

In some embodiments of the phosphine-borane salt of Formula IV, R₁ and R₂=alkoxy and R₃, R₄ and R₅ are each F.

In some embodiments of the phosphine-borane salt of Formula IV, R₁ and R₂=alkoxy and R₃, R₄ and R₅ are each C₆F₅.

In some embodiments of the phosphine-borane salt of Formula IV, R₁=dialkylamino and R₂ and R₃ are both H.

In some embodiments of the phosphine-borane salt of Formula IV, R₁=dialkylamino and R₂, R₃, R₄ and R₅ are each H.

In some embodiments of the phosphine-borane salt of Formula IV, R₁ and R₂=dialkylamino and R₃, R₄ and R₅ are each H.

In some embodiments of the phosphine-borane salt of Formula IV, R₁ and R₂=dialkylamino and R₃, R₄ and R₅ are each F.

In some embodiments of the phosphine-borane salt of Formula IV, R₁ and R₂=dialkylamino and R₃, R₄ and R₅ are each C₆F₅.

In some embodiments of the phosphine-borane salt of Formula IV, R₁=fluoroalkyl and R₂ and R₃ are both H.

In some embodiments of the phosphine-borane salt of Formula IV, R₁=fluoroalkyl and R₂, R₃, R₄ and R₅ are each H.

In some embodiments of the phosphine-borane salt of Formula IV, R₁ and R₂=fluoroalkyl and R₃, R₄ and R₅ are each H.

In some embodiments of the phosphine-borane salt of Formula IV, R₁ and R₂=fluoroalkyl and R₃, R₄ and R₅ are each F.

In some embodiments of the phosphine-borane salt of Formula IV, R₁ and R₂=fluoroalkyl and R₃, R₄ and R₅ are each C₆F₅.

In some embodiments of the phosphine-borane salt of Formula IV, R₁=a group of Formula II and R₂ and R₃ are both H.

In some embodiments of the phosphine-borane salt of Formula IV, R₁=a group of Formula II and R₂, R₃, R₄ and R₅ are each H.

In some embodiments of the phosphine-borane salt of Formula IV, R₁ and R₂=a group of Formula II and R₃, R₄ and R₅ are each H.

In some embodiments of the phosphine-borane salt of Formula IV, R₁ and R₂=a group of Formula II and R₃, R₄ and R₅ are each F.

In some embodiments of the phosphine-borane salt of Formula IV, R₁ and R₂=a group of Formula II and R₃, R₄ and R₅ are each C₆F₅.

In some embodiments of the phosphine-borane salt of Formula IV, R₁=F and R₂ and R₃ are both H.

In some embodiments of the phosphine-borane salt of Formula IV, R₁=F and R₂, R₃, R₄ and R₅ are each H.

In some embodiments of the phosphine-borane salt of Formula IV, R₁ and R₂=F and R₃, R₄ and R₅ are each H.

In some embodiments of the phosphine-borane salt of Formula IV, each of R₁, R₂, R₃, R₄ and R₅ are F.

In some embodiments of the phosphine-borane salt of Formula IV, R₁ and R₂=F and R₃, R₄ and R₅ are each C₆F₅.

In some embodiments of the phosphine-borane salt of Formula IV, R₁=C₆F₅ and R₂ and R₃ are both H.

In some embodiments of the phosphine-borane salt of Formula IV, R₁=C₆F₅ and R₂, R₃, R₄ and R₅ are each H.

In some embodiments of the phosphine-borane salt of Formula IV, R₁ and R₂=C₆F₅ and R₃, R₄ and R₅ are each H.

In some embodiments of the phosphine-borane salt of Formula IV, R₁ and R₂=C₆F₅ and R₃, R₄ and R₅ are each F.

In some embodiments of the phosphine-borane salt of Formula IV, each of R₁, R₂, R₃, R₄ and R₅ are C₆F.

In some embodiments of the phosphine-borane salt of Formula IV, R₁=aryl and R₂ and R₃ are both alkoxy.

In some embodiments of the phosphine-borane salt of Formula IV, R₁=aryl and R₂, R₃, R₄ and R₅ are each alkoxy.

In some embodiments of the phosphine-borane salt of Formula IV, R₁ and R₂=aryl and R₃, R₄ and R₅ are each alkoxy.

In some embodiments of the phosphine-borane salt of Formula IV, R₁ and R₂=aryl and R₃ and R₄ are each alkoxy and are connected by a carbon chain of between 2 and 6 carbons.

In some embodiments of the phosphine-borane salt of Formula IV, R₁=alkyl and R₂ and R₃ are both alkoxy.

In some embodiments of the phosphine-borane salt of Formula IV, R₁ and R₂=alkyl and R₃, R₄ and R₅ are each alkoxy.

In some embodiments of the phosphine-borane salt of Formula IV, R₁ and R₂=alkyl and R₃, R₄ and R₅ are each alkoxy

In some embodiments of the phosphine-borane salt of Formula IV, R₁ and R₂=alkyl and R₃ and R₄ are each alkoxy and are connected by a carbon chain of between 2 and 6 carbons.

In some embodiments of the phosphine-borane salt of Formula IV, R₁, R₂ and R₃ are each alkoxy.

In some embodiments of the phosphine-borane salt of Formula IV, each of R₁, R₂, R₃, R₄ and R₅ are alkoxy.

In some embodiments of the phosphine-borane salt of Formula IV, R₁ and R₂=alkoxy and R₃ and R₄ are each alkoxy and are connected by a carbon chain of between 2 and 6 carbons.

In some embodiments of the phosphine-borane salt of Formula IV, R₁=dialkylamino and R₂ and R₃ are both alkoxy.

In some embodiments of the phosphine-borane salt of Formula IV, R₁=dialkylamino and R₂, R₃, R₄ and R₅ are each alkoxy.

In some embodiments of the phosphine-borane salt of Formula IV, R₁ and R₂=dialkylamino and R₃, R₄ and R₅ are each alkoxy.

In some embodiments of the phosphine-borane salt of Formula IV, R₁ and R₂=dialkylamino and R₃ and R₄ are each alkoxy and are connected by a carbon chain of between 2 and 6 carbons.

In some embodiments of the phosphine-borane salt of Formula IV, R₁=a group of Formula II and R₂ and R₃ are both alkoxy.

In some embodiments of the phosphine-borane salt of Formula IV, R₁=a group of Formula II and R₂, R₃, R₄ and R₅ are each alkoxy.

In some embodiments of the phosphine-borane salt of Formula IV, R₁ and R₂=a group of Formula II and R₃, R₄ and R₅ are each alkoxy.

In some embodiments of the phosphine-borane salt of Formula IV, R₁ and R₂=a group of Formula II and R₃ and R₄ are each alkoxy and are connected by a carbon chain of between 2 and 6 carbons.

In some embodiments of the phosphine-borane salt of Formula IV, R₁=F and R₂ and R₃ are both alkoxy.

In some embodiments of the phosphine-borane salt of Formula IV, R₁=F and R₂, R₃, R₄ and R₅ are each alkoxy.

In some embodiments of the phosphine-borane salt of Formula IV, R₁ and R₂=F and R₃, R₄ and R₅ are each alkoxy.

In some embodiments of the phosphine-borane salt of Formula IV, R₁ and R₂=F and R₃ and R₄ are each alkoxy and are connected by a carbon chain of between 2 and 6 carbons.

In some embodiments of the phosphine-borane salt of Formula IV, R₁=C₆F₅ and R₂ and R₃ are both alkoxy.

In some embodiments of the phosphine-borane salt of Formula IV, R₁=C₆F₅ and R₂, R₃, R₄ and R₅ are each alkoxy.

In some embodiments of the phosphine-borane salt of Formula IV, R₁ and R₂=C₆F₅ and R₃, R₄ and R₅ are each alkoxy.

In some embodiments of the phosphine-borane salt of Formula IV, R₁ and R₂=C₆F₅ and R₃ and R₄ are each alkoxy and are connected by a carbon chain of between 2 and 6 carbons.

In some embodiments of the phosphine-borane salt of Formula IV, R₃ is alkyl.

In some embodiments of the phosphine-borane salt of Formula IV, both R₃ and R₄ are alkyl.

In some embodiments of the phosphine-borane salt of Formula IV, R₃, R₄ and R₅ are each alkyl.

In some embodiments of the phosphine-borane salt of Formula IV, R₃ is fluoroalkyl.

In some embodiments of the phosphine-borane salt of Formula IV, both R₃ and R₄ are fluoroalkyl.

In some embodiments of the phosphine-borane salt of Formula IV, R₃, R₄ and R₅ are each fluoroalkyl.

In some embodiments of the phosphine-borane salt of Formula IV, R₁ is a group of Formula II and each of R′₁, R′₂, R′₃, and R′₄ are methyl groups.

In some embodiments of the phosphine-borane salt of Formula IV, R₁ and R₂ are a group of Formula II and each of R′₁, R′₂, R′₃, and R′₄ are methyl groups.

In some embodiments of the phosphine-borane salt of Formula IV, R₂ and R₈ are joined by one or two additional atoms such that the phosphine-borane forms part of a cyclic 5- or 6-membered ring structure.

In some embodiments of the phosphine-borane salt of Formula IV, M is Li, R₁ and R₂ are phenyl groups, R₃, R₄ and R₅ are each H and the ΔG value of CO₂ binding to the P atom is around −12.8 kcal/mol at 298K, at 1 atm.

In some embodiments of the phosphine-borane salt of Formula IV, M is Li, R₁ and R₂ are methyl groups, R₃, R₄ and R₅ are each H and the ΔG value of CO₂ binding to the P atom is around −23.1 kcal/mol at 298K, at 1 atm.

In some embodiments of the phosphine-borane salt of Formula IV, M is Li, R₁ and R₂ are ethyl groups, R₃, R₄ and R₅ are each H and the ΔG value of CO₂ binding to the P atom is around −24.0 kcal/mol at 298K, at 1 atm.

In some embodiments of the phosphine-borane salt of Formula IV, M is Li, R₁ and R₂ are butyl groups, R₃, R₄ and R₅ are each H and the ΔG value of CO₂ binding to the P atom is around −19.0 kcal/mol at 298K, at 1 atm.

In some embodiments of the phosphine-borane salt of Formula IV, M is Li, R₁ and R₂ are methoxy groups, R₃, R₄ and R₅ are each H and the ΔG value of CO₂ binding to the P atom is around −7.6 kcal/mol at 298K, at 1 atm.

In some embodiments of the phosphine-borane salt of Formula IV, M is Li, R₁ and R₂ are both F, R₃, R₄ and R₅ are each H and the ΔG value of CO₂ binding to the P atom is around −1.3 kcal/mol at 298K, at 1 atm.

In some embodiments of the phosphine-borane salt of Formula IV, M is Li, R₁ and R₂ are both C₆F₅ groups, R₃, R₄ and R₅ are each H and the ΔG value of CO₂ binding to the P atom is around −0.4 kcal/mol at 298K, at 1 atm.

In some embodiments of the phosphine-borane salt of Formula IV, M is Li, R₁ and R₂ are both —N(CH₃)—C═(NCH₃)—N(CH₃)₂ groups, R₃, R₄ and R₅ are each H and the ΔG value of CO₂ binding to the P atom is around −7.4 kcal/mol at 298K, at 1 atm.

In some embodiments of the phosphine-borane salt of Formula IV, M is Li, R₁ and R₂ are both dimethylamino groups, R₃, R₄ and R₅ are each H and the ΔG value of CO₂ binding to the P atom is around −14.3 kcal/mol at 298K, at 1 atm.

In some embodiments of the phosphine-borane salt of Formula IV, M is Na, R₁ and R₂ are methyl groups, R₃, R₄ and R₅ are each H and the ΔG value of CO₂ binding to the P atom is around −19.8 kcal/mol at 298K, at 1 atm.

In some embodiments of the phosphine-borane salt of Formula IV, M is K, R₁ and R₂ are methyl groups, R₃, R₄ and R₅ are each H and the ΔG value of CO₂ binding to the P atom is around −17.9 kcal/mol at 298K, at 1 atm.

In some embodiments of the phosphine-borane salt of Formula IV, M is Na, R₁ and R₂ are phenyl groups, R₃, R₄ and R₅ are each H and the ΔG value of CO₂ binding to the P atom is around −8.8 kcal/mol at 298K, at 1 atm.

In some embodiments of the phosphine-borane salt of Formula IV, M is K, R₁ and R₂ are phenyl groups, R₃, R₄ and R₅ are each H and the ΔG value of CO₂ binding to the P atom is around −4.9 kcal/mol at 298K, at 1 atm.

In some embodiments of the phosphine-borane salt of Formula IV, M is Li, R₁ is a phenyl group, R₂ and R₃ are connected by a 3 carbon chain, R₄ and R₅ are each H and the ΔG value of CO₂ binding to the P atom is around −20.2 kcal/mol at 298K, at 1 atm.

In some embodiments of the phosphine-borane salt of Formula IV, M is Li, R₁ is a phenyl group, R₂ and R₃ are connected by a 4 carbon chain, R₄ and R₅ are each H and the ΔG value of CO₂ binding to the P atom is around −13.4 kcal/mol at 298K, at 1 atm.

In some embodiments of the phosphine-borane salt of Formula IV, M is Li, R₁, R₂, R₃ and R₄ are methyl groups and R₅ is H and the ΔG value of CO₂ binding to the P atom is around −22.3 kcal/mol at 298K, at 1 atm.

In some embodiments of the phosphine-borane salt of Formula IV, the compound further comprises part of the repeat unit of a polymer where the polymer chain is connected through any one of R₁ to R₆.

In some embodiments of the phosphine-borane salt of Formula IV, the compound further comprises part of the repeat unit of a polymer, where the polymer chain is connected through repeated B—P bonds. An exemplary structure is reported in Formula IVa)

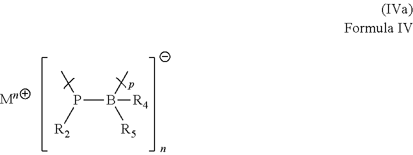

where the polymer is comprised of P—B repeat units such that R₁ and R₃ are B and P respectively; p is the number of P—B repeat units; “n” units of polymer to balance the n⁺ charges required by the identity of M.

In some embodiments of the phosphine-borane salt of Formula IV, R₁ is B, such that the structure of Formula IV comprises a bis-phosphine-borane.

In some embodiments of the phosphine-borane salt of Formula IV, ΔG for CO₂ binding to the resulting deprotonated complex is <−5 kcal/mol at 298K, 1 atm.

In some embodiments of the phosphine-borane salt of Formula IV, ΔG for CO₂ binding to the resulting deprotonated complex is 0<ΔG≤−5 kcal/mol at 298K, 1 atm.

In some embodiments, phosphorous boron-bonded compounds of the disclosure of Formula I, Formula III and/or Formula IV can be used to capture carbon dioxide.

The term “carbon dioxide” or “CO₂”, as referred to herein, is a chemical compound comprising one carbon atom covalently double bonded to two oxygen atoms, typically present as a colorless, odorless gas under standard temperature and pressure conditions

The term “capturing” or “capture” of CO₂, as used in this application, refers to any process, method, or system by which carbon dioxide is selectively removed, separated, or extracted from a gas stream, ambient air, or other source, and subsequently collected, sequestered, or otherwise prevented from being released into the atmosphere. Such processes may include, but are not limited to, physical or chemical absorption, adsorption, membrane separation, cryogenic separation, or other techniques designed to isolate and retain CO₂ for subsequent storage, utilization, or conversion.

The captured CO₂ can be stored in geological formations, utilized as a feedstock for chemical synthesis, or otherwise processed to mitigate greenhouse gas emissions and address climate as will be understood by a skilled person

Therefore, in the context of this disclosure, “CO₂ capturing” encompasses all technologies, materials, devices, and methods that effectuate the removal and retention of carbon dioxide from any source, such to industrial flue gases, natural gas streams, or ambient air from a target environment

The term “target environment,” as used herein, refers to any physical, chemical, biological, or operational setting, location, or medium in which a process, system, device, or composition is intended to function, interact, or achieve a desired effect or outcome. Exemplary target environments comprise natural or artificial surroundings such as atmospheric air, aquatic systems, terrestrial landscapes, industrial facilities, laboratory conditions, or any other milieu relevant to the application of the invention. A target environment can be characterized by specific parameters, such as temperature, pressure, composition, or the presence of particular substances, which are pertinent to the operation, efficacy, or an intended use of the environment.

In some embodiment, the target environment can comprise the Earth's atmosphere, where carbon dioxide exists as a trace gas at approximately 0.041% by volume. In the atmosphere, CO₂ is distributed globally, influenced by natural cycles such as photosynthesis, respiration, and the decomposition of organic matter, as well as by anthropogenic emissions from industrial activity, transportation, and energy production. The atmosphere's vastness and the dilute nature of CO₂ present unique challenges for removal, often requiring advanced technologies to extract even small quantities efficiently as will be understood by a skilled person.

In some embodiment, the target environment can comprise an ocean. Oceans act as a dynamic reservoir, absorbing and storing large amounts of CO₂ from the atmosphere through both physical dissolution and biological processes. The interaction between atmospheric CO₂ and seawater not only affects global carbon cycles but also leads to changes in ocean chemistry, such as acidification. The removal of CO₂ from oceanic environments is complex, given the vast volume of water and the interplay between dissolved inorganic carbon species.

In some embodiment, the target environment can comprise terrestrial and biological environments which can be major domains for CO₂ presence and cycling. In soils and forests, CO₂ is both produced and absorbed through processes like plant respiration, microbial activity, and organic matter decomposition. Forests and other vegetated landscapes act as carbon sinks, temporarily storing atmospheric CO₂ in plant biomass and soil organic matter. However, land-use changes, deforestation, and agricultural practices can release significant amounts of CO₂ back into the atmosphere.

In some embodiment, the target environment can comprise industrial environments, particularly those associated with the combustion of fossil fuels and the processing of minerals and chemicals, represent concentrated sources of CO₂ emissions. Power plants, cement factories, steel mills, and chemical refineries emit CO₂ in flue gases and process streams, often at much higher concentrations than found in ambient air. These point sources are considered highly desirable targets for CO₂ removal because the elevated concentrations make capture and subsequent storage or utilization more efficient and cost-effective.

In some embodiment, the target environment can comprise geological environments, such as underground reservoirs, saline aquifers, and basalt formations, are not only natural sources of CO₂ (through volcanic activity and gas seeps) but also serve as potential sites for the long-term storage of captured CO₂. These subsurface environments offer the capacity to sequester large volumes of CO₂ safely and permanently, thereby preventing its release into the atmosphere.

In some embodiment, the target environment can comprise waste management environments, including landfills and sites of organic waste decomposition, produce CO₂ (often alongside methane) as a byproduct of microbial breakdown of organic materials. These environments are increasingly targeted for gas capture systems to reduce greenhouse gas emissions and recover energy.

In some embodiments, methods directed to capture carbon dioxide from a target environment comprise one or more phosphorous boron-bonded compounds of the disclosure selected from one or more phosphine-borane of Formula I, one or more cyclic phosphine-borane of Formula III and one or more phosphine-borane salt of Formula IV can be reacted with CO₂ obtain a phosphino-borane-carbon dioxide complex Formula V

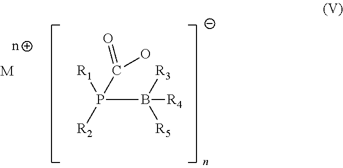

wherein
n is an integer from 1 to 3
R₁, R₂, R₃, R₄ and R₅ are each independently H, F or a substituted or unsubstituted alkyl, aryl, alkoxy, aryloxy or dialkylamino group, or group of Formula II

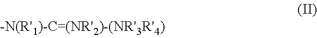

in which R′₁, R′₂, R′₃, and R′₄ are independently a substituted or unsubstituted alkyl or aryl group, and
M is Li, Na, K, Rb, Cs or tetraalkylammonium when n=1; Mg, Ca, Sr, Ba when n=2 or Al when n=3.

In some embodiments of the phosphinoborane-carbon dioxide complex of Formula V, the substituted or unsubstituted alkyl or aryl are substituted or unsubstituted fluoroalkyl or fluoroaryl.

In some embodiments of the phosphinoborane-carbon dioxide complex of Formula V, R₁=aryl and R₂ and R₃ are both H.

In some embodiments of the phosphinoborane-carbon dioxide complex of Formula V, R₁=aryl and R₂, R₃, R₄ and R₅ are each H.

In some embodiments of the phosphinoborane-carbon dioxide complex of Formula V, R₁ and R₂=aryl and R₃, R₄ and R₅ are each H.

In some embodiments of the phosphinoborane-carbon dioxide complex of Formula V, R₁ and R₂=aryl and R₃, R₄ and R₅ are each F.

In some embodiments of the phosphinoborane-carbon dioxide complex of Formula V, R₁ and R₂=aryl and R₃, R₄ and R₅ are each C₆F₄.

In some embodiments of the phosphinoborane-carbon dioxide complex of Formula V, R₁=alkyl and R₂ and R₃ are both H.

In some embodiments of the phosphinoborane-carbon dioxide complex of Formula V, R₁ and R₂=alkyl and R₃, R₄ and R₅ are each H.

In some embodiments of the phosphinoborane-carbon dioxide complex of Formula V, R₁ and R₂=alkyl and R₃, R₄ and R₅ are each F.

In some embodiments of the phosphinoborane-carbon dioxide complex of Formula V, R₁ and R₂=alkyl and R₃, R₄ and R₅ are each C₆F₄.

In some embodiments of the phosphinoborane-carbon dioxide complex of Formula V, R₁=alkoxy and R₂ and R₃ are both H.

In some embodiments of the phosphinoborane-carbon dioxide complex of Formula V, R₁=alkoxy and R₂, R₃, R₄ and R₅ are each H.

In some embodiments of the phosphinoborane-carbon dioxide complex of Formula V, R₁ and R₂=alkoxy and R₃, R₄ and R₅ are each H.

In some embodiments of the phosphinoborane-carbon dioxide complex of Formula V, R₁ and R₂=alkoxy and R₃, R₄ and R₅ are each F.

In some embodiments of the phosphinoborane-carbon dioxide complex of Formula V, R₁ and R₂=alkoxy and R₃, R₄ and R₅ are each C₆F₄.

In some embodiments of the phosphinoborane-carbon dioxide complex of Formula V, R₁=dialkylamino and R₂ and R₃ are both H.

In some embodiments of the phosphinoborane-carbon dioxide complex of Formula V, R₁=dialkylamino and R₂, R₃, R₄ and R₅ are each H.

In some embodiments of the phosphinoborane-carbon dioxide complex of Formula V, R₁ and R₂=dialkylamino and R₃, R₄ and R₅ are each H.

In some embodiments of the phosphinoborane-carbon dioxide complex of Formula V, R₁ and R₂=dialkylamino and R₃, R₄ and R₅ are each F.

In some embodiments of the phosphinoborane-carbon dioxide complex of Formula V, R₁ and R₂=dialkylamino and R₃, R₄ and R₅ are each C₆F₄.

In some embodiments of the phosphinoborane-carbon dioxide complex of Formula V, R₁=fluoroalkyl and R₂ and R₃ are both H.

In some embodiments of the phosphinoborane-carbon dioxide complex of Formula V, R₁=fluoroalkyl and R₂, R₃, R₄ and R₅ are each H.

In some embodiments of the phosphinoborane-carbon dioxide complex of Formula V, R₁ and R₂=fluoroalkyl and R₃, R₄ and R₅ are each H.

In some embodiments of the phosphinoborane-carbon dioxide complex of Formula V, R₁ and R₂=fluoroalkyl and R₃, R₄ and R₅ are each F.

In some embodiments of the phosphinoborane-carbon dioxide complex of Formula V, R₁ and R₂=fluoroalkyl and R₃, R₄ and R₅ are each C₆F₄.

In some embodiments of the phosphinoborane-carbon dioxide complex of Formula V, R₁=a group of Formula II and R₂ and R₃ are both H.

In some embodiments of the phosphinoborane-carbon dioxide complex of Formula V, R₁=a group of Formula II and R₂, R₃, R₄ and R₅ are each H.

In some embodiments of the phosphinoborane-carbon dioxide complex of Formula V, R₁ and R₂=a group of Formula II and R₃, R₄ and R₅ are each H.

In some embodiments of the phosphinoborane-carbon dioxide complex of Formula V, R₁ and R₂=a group of Formula II and R₃, R₄ and R₅ are each F.

In some embodiments of the phosphinoborane-carbon dioxide complex of Formula V, R₁ and R₂=a group of Formula II and R₃, R₄ and R₅ are each C₆F₄.

In some embodiments of the phosphinoborane-carbon dioxide complex of Formula V, R₁=F and R₂ and R₃ are both H.

In some embodiments of the phosphinoborane-carbon dioxide complex of Formula V, R₁=F and R₂, R₃, R₄ and R₅ are each H.

In some embodiments of the phosphinoborane-carbon dioxide complex of Formula V, R₁ and R₂=F and R₃, R₄ and R₅ are each H.

In some embodiments of the phosphinoborane-carbon dioxide complex of Formula V, each of R₁, R₂, R₃, R₄ and R₅ are F.

In some embodiments of the phosphinoborane-carbon dioxide complex of Formula V, R₁ and R₂=F and R₃, R₄ and R₅ are each C₆F₄.

In some embodiments of the phosphinoborane-carbon dioxide complex of Formula V, R₁=C₆F₅ and R₂ and R₃ are both H.

In some embodiments of the phosphinoborane-carbon dioxide complex of Formula V, R₁=C₆F₅ and R₂, R₃, R₄ and R₅ are each H.

In some embodiments of the phosphinoborane-carbon dioxide complex of Formula V, R₁ and R₂=C₆F₅ and R₃, R₄ and R₅ are each H.

In some embodiments of the phosphinoborane-carbon dioxide complex of Formula V, R₁ and R₂=C₆F₅ and R₃, R₄ and R₅ are each F.

In some embodiments of the phosphinoborane-carbon dioxide complex of Formula V, each of R₁, R₂, R₃, R₄ and R₅ are C₆F₄.

In some embodiments of the phosphinoborane-carbon dioxide complex of Formula V, R₁=aryl and R₂ and R₃ are both alkoxy.

In some embodiments of the phosphinoborane-carbon dioxide complex of Formula V, R₁=aryl and R₂, R₃, R₄ and R₅ are each alkoxy.

In some embodiments of the phosphinoborane-carbon dioxide complex of Formula V, R₁ and R₂=aryl and R₃, R₄ and R₅ are each alkoxy.

In some embodiments of the phosphinoborane-carbon dioxide complex of Formula V, R₁ and R₂=aryl and R₃ and R₄ are each alkoxy and are connected by a carbon chain of between 2 and 6 carbons.

In some embodiments of the phosphinoborane-carbon dioxide complex of Formula V, R₁=alkyl and R₂ and R₃ are both alkoxy.

In some embodiments of the phosphinoborane-carbon dioxide complex of Formula V, R₁ and R₂=alkyl and R₃, R₄ and R₅ are each alkoxy.

In some embodiments of the phosphinoborane-carbon dioxide complex of Formula V, R₁ and R₂=alkyl and R₃, R₄ and R₅ are each alkoxy

In some embodiments of the phosphinoborane-carbon dioxide complex of Formula V, R₁ and R₂=alkyl and R₃ and R₄ are each alkoxy and are connected by a carbon chain of between 2 and 6 carbons.

In some embodiments of the phosphinoborane-carbon dioxide complex of Formula V, R₁, R₂ and R₃ are each alkoxy.

In some embodiments of the phosphinoborane-carbon dioxide complex of Formula V, each of R₁, R₂, R₃, R₄ and R₅ are alkoxy.

In some embodiments of the phosphinoborane-carbon dioxide complex of Formula V, R₁ and R₂=alkoxy and R₃ and R₄ are each alkoxy and are connected by a carbon chain of between 2 and 6 carbons.

In some embodiments of the phosphinoborane-carbon dioxide complex of Formula V, R₁=dialkylamino and R₂ and R₃ are both alkoxy.

In some embodiments of the phosphinoborane-carbon dioxide complex of Formula V, R₁=dialkylamino and R₂, R₃, R₄ and R₅ are each alkoxy.

In some embodiments of the phosphinoborane-carbon dioxide complex of Formula V, R₁ and R₂=dialkylamino and R₃, R₄ and R₅ are each alkoxy.

In some embodiments of the phosphinoborane-carbon dioxide complex of Formula V, R₁ and R₂=dialkylamino and R₃ and R₄ are each alkoxy and are connected by a carbon chain of between 2 and 6 carbons.

In some embodiments of the phosphinoborane-carbon dioxide complex of Formula V, R₁=a group of Formula II and R₂ and R₃ are both alkoxy.

In some embodiments of the phosphinoborane-carbon dioxide complex of Formula V, R₁=a group of Formula II and R₂, R₃, R₄ and R₅ are each alkoxy.

In some embodiments of the phosphinoborane-carbon dioxide complex of Formula V, R₁ and R₂=a group of Formula II and R₃, R₄ and R₅ are each alkoxy.

In some embodiments of the phosphinoborane-carbon dioxide complex of Formula V, R₁ and R₂=a group of Formula II and R₃ and R₄ are each alkoxy and are connected by a carbon chain of between 2 and 6 carbons.

In some embodiments of the phosphinoborane-carbon dioxide complex of Formula V, R₁=F and R₂ and R₃ are both alkoxy.

In some embodiments of the phosphinoborane-carbon dioxide complex of Formula V, R₁=F and R₂, R₃, R₄ and R₅ are each alkoxy.

In some embodiments of the phosphinoborane-carbon dioxide complex of Formula V, R₁ and R₂=F and R₃, R₄ and R₅ are each alkoxy.

In some embodiments of the phosphinoborane-carbon dioxide complex of Formula V, R₁ and R₂=F and R₃ and R₄ are each alkoxy and are connected by a carbon chain of between 2 and 6 carbons.

In some embodiments of the phosphinoborane-carbon dioxide complex of Formula V, R₁=C₆F₅ and R₂ and R₃ are both alkoxy.

In some embodiments of the phosphinoborane-carbon dioxide complex of Formula V, R₁=C₆F₅ and R₂, R₃, R₄ and R₅ are each alkoxy.

In some embodiments of the phosphinoborane-carbon dioxide complex of Formula V, R₁ and R₂=C₆F₅ and R₃, R₄ and R₅ are each alkoxy.

In some embodiments of the phosphinoborane-carbon dioxide complex of Formula V, R₁ and R₂=C₆F₅ and R₃ and R₄ are each alkoxy and are connected by a carbon chain of between 2 and 6 carbons.

In some embodiments of the phosphinoborane-carbon dioxide complex of Formula V, R₃ is alkyl.

In some embodiments of the phosphinoborane-carbon dioxide complex of Formula V, both R₃ and R₄ are alkyl.

In some embodiments of the phosphinoborane-carbon dioxide complex of Formula V, R₃, R₄ and R₅ are each alkyl.

In some embodiments of the phosphinoborane-carbon dioxide complex of Formula V, R₃ is fluoroalkyl.

In some embodiments of the phosphinoborane-carbon dioxide complex of Formula V, both R₃ and R₄ are fluoroalkyl.

In some embodiments of the phosphinoborane-carbon dioxide complex of Formula V, R₃, R₄ and R₅ are each fluoroalkyl.

In some embodiments of the phosphinoborane-carbon dioxide complex of Formula V, R₁ is a group of Formula II and each of R′₁, R′₂, R′₃, and R′₄ are methyl groups.

In some embodiments of the phosphinoborane-carbon dioxide complex of Formula V, R₁ and R₂ are a group of Formula II and each of R′₁, R′₂, R′₃, and R′₄ are methyl groups.

In some embodiments of the phosphinoborane-carbon dioxide complex of Formula V, R₂ and R₅ are joined by one or two additional atoms such that the phosphine-borane forms part of a cyclic 5- or 6-membered ring structure.

In some embodiments of the phosphinoborane-carbon dioxide complex of Formula V, M is Li, R₁ and R₂ are phenyl groups, R₃, R₄ and R₅ are each H

In some embodiments of the phosphinoborane-carbon dioxide complex of Formula V, M is Li, R₁ and R₂ are methyl groups, R₃, R₄ and R₅ are each H

In some embodiments of the phosphinoborane-carbon dioxide complex of Formula V, M is Li, R₁ and R₂ are ethyl groups, R₃, R₄ and R₅ are each H

In some embodiments of the phosphinoborane-carbon dioxide complex of Formula V, M is Li, R₁ and R₂ are butyl groups, R₃, R₄ and R₅ are each H

In some embodiments of the phosphinoborane-carbon dioxide complex of Formula V, M is Li, R₁ and R₂ are methoxy groups, R₃, R₄ and R₅ are each H

In some embodiments of the phosphinoborane-carbon dioxide complex of Formula V, M is Li, R₁ and R₂ are both F, R₃, R₄ and R₅ are each H

In some embodiments of the phosphinoborane-carbon dioxide complex of Formula V, M is Li, R₁ and R₂ are both C₆F₅ groups, R₃, R₄ and R₅ are each H

In some embodiments of the phosphinoborane-carbon dioxide complex of Formula V, M is Li, R₁ and R₂ are both —N(CH₃)—C═(NCH₃)—N(CH₃)₂ groups, R₃, R₄ and R₅ are each H

In some embodiments of the phosphinoborane-carbon dioxide complex of Formula V, M is Li, R₁ and R₂ are both dimethylamino groups, R₃, R₄ and R₅ are each H

In some embodiments of the phosphinoborane-carbon dioxide complex of Formula V, M is Na, R₁ and R₂ are methyl groups, R₃, R₄ and R₅ are each H

In some embodiments of the phosphinoborane-carbon dioxide complex of Formula V, M is K, R₁ and R₂ are methyl groups, R₃, R₄ and R₅ are each H

In some embodiments of the phosphinoborane-carbon dioxide complex of Formula V, M is Na, R₁ and R₂ are phenyl groups, R₃, R₄ and R₅ are each H

In some embodiments of the phosphinoborane-carbon dioxide complex of Formula V, M is K, R₁ and R₂ are phenyl groups, R₃, R₄ and R₅ are each H

In some embodiments of the phosphinoborane-carbon dioxide complex of Formula V, M is Li, R₁ is a phenyl group, R₂ and R₃ are connected by a 3 carbon chain, R₄ and R₅ are each H

In some embodiments of the phosphinoborane-carbon dioxide complex of Formula V, M is Li, R₁ is a phenyl group, R₂ and R₃ are connected by a 4 carbon chain, R₄ and R₅ are each H

In some embodiments of the phosphinoborane-carbon dioxide complex of Formula V, M is Li, R₁, R₂, R₃ and R₄ are methyl groups and R₅ is H

In some embodiments of the phosphinoborane-carbon dioxide complex of Formula V, R₁ and R₂ are CH₃, C₂H₅ or C₄H₉, R₃, R₄ and R₅ are each H and M is Na, K, Rb, Cs, tetraalkylammonium, Mg, Ca, Sr, Ba or Al.

In some embodiments of the phosphinoborane-carbon dioxide complex of Formula V, R₁ and R₂ are phenyl groups, R₃, R₄ and R₅ are each H and M is Na, K, Rb, Cs, tetraalkylammonium, Mg, Ca, Sr, Ba or Al.

In some embodiments of the phosphinoborane-carbon dioxide complex of Formula V, R₁ and R₂ are dialkylamino groups, R₃, R₄ and R₅ are each H and M is Na, K, Rb, Cs, tetraalkylammonium, Mg, Ca, Sr, Ba or Al.

In some embodiments of the phosphinoborane-carbon dioxide complex of Formula V, wherein the compound further comprises part of the repeat unit of a polymer where the polymer chain is connected through any one of R₁ to R₆. An exemplary structure is reported as Formula IVb) below

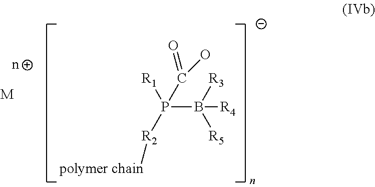

where R₂ is linked to a polymer main chain.

In some embodiments of the phosphinoborane-carbon dioxide complex of Formula V, wherein the compound further comprises part of the repeat unit of a polymer, where the polymer chain is connected through repeated B—P bonds.

An exemplary structure is reported in Formula IV(c) below

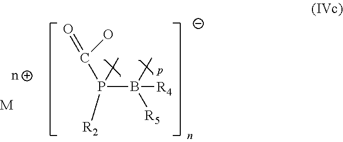

where the polymer is comprised of P—B repeat units such that R₁ and R₃ are B and P respectively; p is the number of P—B repeat units; “n” units of polymer to balance the n+ charges required by the identity of M

In some embodiments of the phosphinoborane-carbon dioxide complex of Formula V, R₁ is B, such that the structure of Formula I comprises a bis-phosphine-borane.

In some embodiments formation of the phosphino-borane-carbon dioxide complex Formula V can also be confirmed by computational validation as will be understood by a skilled person upon reading of the present disclosure.

In some embodiments the method for capturing carbon dioxide from a target environment using one or more phosphorous boron-bonded compounds of the disclosure. comprises: contacting the target environment with the at least one phosphorous boron-bonded compound of the disclosure selected from a phosphine-borane of Formula I, a cyclic phosphine-borane of Formula III and a phosphine-borane salt of Formula IV, to obtain a phosphino-borane-carbon dioxide complex which is the phosphino-borane-carbon dioxide complex Formula V of the present disclosure.

In embodiments where the one or more phosphorous boron-bonded compounds of the disclosure comprises one or more phosphine-borane of Formula I and/or one or more cyclic phosphine-borane of Formula III the contacting is performed in presence of a base to convert the phosphine borane compounds in a phosphine-borane salt of Formula IV,

The term “base” in the context of the disclosure indicates a substance (typically a strong Lewis base or Brønsted base) that abstracts a proton (H⁺) or hydride (H⁻) from the phosphorous boron-bonded compounds, generating an anionic phosphide-borane species that pairs with a cationic counterion to form a salt. This reaction exploits the acidity of P—H or B—H bonds in phosphine-boranes, enabling deprotonation and salt formation as will be understood by a skilled person. Bases in the sense of the disclosure can accordingly enable salt formation by deprotonation, hydride abstraction, or stabilization of zwitterionic species, with counterions ([BArF₄]⁻, [OTf]⁻) playing a critical role in solubility and reactivity as will also be understood by a skilled person

Exemplary bases which can used in combination with phosphorous boron-bonded compounds of the disclosure in methods and systems to capture CO2 of the disclosure comprise alkali metal amides (e.g., LiHMDS, NaNH₂, LDA) which are capable of abstract protons from P—H bonds to generate phosphide-borane anions, forming salts like [R₂P⁻·BH₃·Li⁺], cyclic alkyl amino carbenes (CAACs) which act as stoichiometric hydrogen acceptors, enabling dehydrogenation and salt formation via zwitterionic intermediates, e.g., [CAAC(H)]⁺[RPH(BH₃)]⁻), alkali metal alkoxides (e.g., LiOtBu)) tertiary amines (e.g., Et₃N, TMEDA) Grignard reagents (e.g., RMgX) which are capable to deprotonate phosphine-boranes (RPH₂·BH₃) to form carbanion complexes, and ionic bases (e.g., LiOTf)

In some embodiments the base can comprise one or more amide, alkoxide, hydroxide, amide, aryloxide (and in particular phenoxide), amine or silazide bases

In some embodiments the base can be lithium tert-butoxide and the concentration of the phosphino-borane composition is between 1M and 0.001 M.

In some embodiments the base can be sodium tert-butoxide and the concentration of the phosphino-borane composition is between 1M and 0.001 M.

In some embodiments the base can be is sodium amide and the concentration of the phosphino-borane composition is between 1M and 0.001 M.

In some embodiments the base can be potassium hydroxide and the concentration of the phosphino-borane composition is between 1M and 0.001 M.

In some embodiments the base can be tetramethylammonium hydroxide and the concentration of the phosphino-borane composition is between 1M and 0.001 M.

In some embodiments the base can be lithium bis(trimethylsilyl)amide and the concentration of the phosphino-borane composition is between 1M and 0.001 M.

In embodiments of methods of the disclosure directed to the capture of CO2 where the one or more phosphorous boron-bonded compounds of the disclosure comprises one or more phosphine-borane of Formula I and/or one or more cyclic phosphine-borane of Formula III one or more bases can be present in a reaction environment or added with the one or more phosphorous boron-bonded compounds for simultaneous combined or sequential use to perform the CO2 capture according to methods and systems of the disclosure. In some embodiments, of methods directed to capture carbon dioxide from a target environment, the base can be presented in a reaction environment dissolved in a solvent.

In some embodiments, of methods directed to capture carbon dioxide from a target environment the CO₂ is dissolved in a solvent.

In some embodiments, of methods directed to capture carbon dioxide from a target environment the CO₂ is in the gas phase.

In some embodiments, of methods directed to capture carbon dioxide from a target environment the CO₂ is present in a flowing stream of gas introduced to the composition in solvent.

In some embodiments, of methods directed to capture carbon dioxide from a target environment the CO₂ is present in a static mixture of gases.

In some embodiments, of methods directed to capture carbon dioxide from a target environment the CO₂ is present in a flowing stream of gas introduced to the composition in solvent.

In some embodiments, of methods directed to capture carbon dioxide from a target environment the CO₂ is present in a static mixture of gases.

The time and extent to which a phosphine-borane of Formula I, a cyclic phosphine-borane of Formula III and a phosphine-borane salt of Formula IV, binds a CO2, as well as the reversibility of the interaction, is affected by the electronic and steric properties of substituents on phosphine-borane of Formula I, a cyclic phosphine-borane of Formula III and a phosphine-borane salt of Formula IV and the nature of counterions present for a phosphine-borane salt of Formula IV.

In some embodiment, where irreversible binding of a phosphine-borane salt of Formula I with CO₂ the ΔG for CO₂ binding to the resulting deprotonated complex of Formula IV is <−5 kcal/mol at 298K and 1 atm, preferably −5<ΔG<−30 kcal/mol at 298K and 1 atm, more preferably −7<ΔG<−25 kcal/mol at 298K and 1 atm, more preferably −13<ΔG<−25 kcal/mol at 298K and 1 atm, −19<ΔG<−24 kcal/mol at 298K and 1 atm and −13<ΔG<−21 kcal/mol at 298K and 1 atm., depending on the type of phosphine-borane salt and the desired irreversibility.

In some embodiment, where eversible binding of a phosphine-borane salt of Formula I with CO₂ the ΔG for CO₂ binding to the resulting deprotonated complex of Formula IV, is −0<ΔG<−5 kcal/mol at 298K and 1 atm, −0.4<ΔG<−5 kcal/mol at 298K and 1 atm, −0.4<ΔG<−4.9 kcal/mol at 298K and 1 atm, −1.3<ΔG<−4.9 kcal/mol at 298K and 1 atm, −0.4<ΔG<−1.3 kcal/mol at 298K and 1 atm, depending on the type of phosphine-borane salt and the desired reversibility.

Configuration of the phosphine-borane of Formula I, a cyclic phosphine-borane of Formula III and a phosphine-borane salt of Formula IV, can be engineered to obtain reversible or irreversible CO₂ capture as will be understood by a skilled person upon reading of the disclosure.

In particular, in phosphine-borane of Formula I, a cyclic phosphine-borane of Formula III and a phosphine-borane salt of Formula IV, electron withdrawing substituents decrease the overall ΔG of CO₂ binding thus lowering the reversibility of the CO₂ binding. This is a result of decreased electron-density on the nucleophile, reducing the affinity for CO₂ capture via nucleophilic attack either via inductive or resonance effects. Examples of electron-withdrawing compounds are —CF3, —F, CH2OH, —Ph, and additional electron-withdrawing groups identifiable by a skilled person upon reading of the present disclosure.

In phosphine-borane of Formula I, a cyclic phosphine-borane of Formula III and a phosphine-borane salt of Formula IV, electron donating substituents increase the overall ΔG of CO2 binding thus increasing the reversibility of the CO2 binding. Electron donating groups funnel electron-density towards the center parent moiety where nucleophilic attack occurs, increasing the reactivity of the reactant and hence increase the propensity of CO2 capture. Examples of electron-donating compounds are —CNMe2, -t-Bu, -Et, -Me, —NH2, and additional electron donating groups identifiable by a skilled person upon reading of the present disclosure.

Additionally, in phosphine-borane salt of Formula IV, smaller, more positively charged counterions stabilize the product state, increase the value of ΔG and thus increase reversibility. This includes Li⁺, Na⁺, and additional counterions identifiable by a skilled person upon reading of the present disclosure. Larger, more diffuse counterions are not able to as favorably stabilize the product, leading to decreases in ΔG and thus to a decrease in reversibility as would be understood by skilled person upon reading of the present disclosure.

Thus, in embodiments of methods of the disclosure directed to the capture of CO₂ strategic configuration of substituents and counterions allows precise control over binding strength (ΔG) and practical reversibility, enabling tailored applications in catalysis, sensing, or gas storage as would be understood by skilled person upon reading of the present disclosure.

In some embodiments, where irreversible CO₂ capture is desired, in phosphine-borane of Formula I, a cyclic phosphine-borane of Formula III and a phosphine-borane salt of Formula IV, Mⁿ⁺=Li, Na, Mg, Al; substituents, and especially R₁ and R₂ are electron donating groups preferably in phosphine-borane of Formula I, a cyclic phosphine-borane of Formula III and a phosphine-borane salt of Formula IV, R₁ and R₂=alkyl>comprising a cyclic P—B>dimethylamino>alkoxy>guanidyl (Formula II), and more preferably R₃, R₄, R₅=alkyl˜H>aryl>fluoralkyl, fluoraryl, reported in order of increasingly more negative ΔG as will be understood by a skilled person.

In some embodiments, where irreversible CO₂ capture is desired, in phosphine-borane of Formula I, a cyclic phosphine-borane of Formula III and a phosphine-borane salt of Formula IV, R₁ and R₂=C₂H₅ and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=C₄H₉ and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=N(CH₃)₂ and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=OCH₃ and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=—N(CH₃)—C═(NCH₃)—N(CH₃)₂ and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=CH₃ and R₃, R₄ and R₅=CH₃.

In some embodiments, where irreversible CO₂ capture is desired, in phosphine-borane of Formula I, a cyclic phosphine-borane of Formula III and a phosphine-borane salt of Formula IV, R₁ and R₂=CH₃, and R₃, R₄ and R₅=H.

In some embodiments, where irreversible CO₂ capture is desired, in phosphine-borane of Formula I, a cyclic phosphine-borane of Formula III and a phosphine-borane salt of Formula IV, R₁ and R₂=Ph and R₃, R₄ and R₅=H.

In some embodiments, where reversible CO₂ capture is desired, in phosphine-borane of Formula I, a cyclic phosphine-borane of Formula III and a phosphine-borane salt of Formula IV, M⁺=K, Rb, Cs, tetraalkylammonium; substituents=EWG, especially R₁ and R₂=Electron withdrawing groups. Preferably in phosphine-borane of Formula I, a cyclic phosphine-borane of Formula III and a phosphine-borane salt of Formula IV, R₁ and R₂=aryl>F>fluoroaryl˜fluoroalkyl, more preferably R₃, R₄, R₅=H>aryl>fluoralkyl, fluoraryl, reported in order of increasingly more negative ΔG as will be understood by a skilled person.

In some embodiments, where reversible CO₂ capture is desired, in phosphine-borane of Formula I, a cyclic phosphine-borane of Formula III and a phosphine-borane salt of Formula IV, R₃, R₄, R₅ are alkyl-H, aryl, fluoralkyl, and/or fluoroaryl groups.

In some embodiments, where reversible CO₂ capture is desired, in phosphine-borane of Formula I, a cyclic phosphine-borane of Formula III and a phosphine-borane salt of Formula IV, R₁ and R₂=F and R₃, R₄ and R₅=H.

In some embodiments, where reversible CO₂ capture is desired, in phosphine-borane of Formula I, a cyclic phosphine-borane of Formula III and a phosphine-borane salt of Formula IV, R₁ and R₂=C₆F₅ and R₃, R₄ and R₅=H.

In some embodiments, where reversible CO₂ capture is desired, in phosphine-borane of Formula I, a cyclic phosphine-borane of Formula III and a phosphine-borane salt of Formula IV, M=Li, Na, K.

In some embodiments, where reversible CO₂ capture is desired, in phosphine-borane of Formula I, a cyclic phosphine-borane of Formula III and a phosphine-borane salt of Formula IV, R₁ and R₂=Ph and R₃, R₄ and R₅=H.

In some embodiments, where reversible CO₂ capture is desired, in phosphine-borane of Formula I, a cyclic phosphine-borane of Formula III and a phosphine-borane salt of Formula IV, M=K, tetraalkylammonium.

In some embodiments, where reversible CO₂ capture is desired, in phosphine-borane of Formula I, a cyclic phosphine-borane of Formula III and a phosphine-borane salt of Formula IV, R₁ and R₂=OCH₃ and R₃, R₄ and R₅=H.

In some embodiments, where reversible CO₂ capture is desired, in phosphine-borane of Formula I, a cyclic phosphine-borane of Formula III and a phosphine-borane salt of Formula IV, R₁ and R₂=—N(CH₃)—C═(NCH₃)—N(CH₃)₂ and R₃, 30 The method of claim 18, wherein R₄ and R₅=H.

In some embodiments, where reversible CO₂ capture is desired, in phosphine-borane of Formula I, a cyclic phosphine-borane of Formula III and a phosphine-borane salt of Formula IV, R₁ and R₂=CH₃ and R₃, R₄ and R₅=CH₃.

In some embodiments, where reversible CO₂ capture is desired, in phosphine-borane of Formula I, a cyclic phosphine-borane of Formula III and a phosphine-borane salt of Formula IV, M=tetraalkylammonium.

In some embodiments, where reversible CO₂ capture is desired, in phosphine-borane of Formula I, a cyclic phosphine-borane of Formula III and a phosphine-borane salt of Formula IV, n=1, R₁=Ph, R₄ and R₅=H.

In embodiments of the method for capturing carbon dioxide from a target environment herein described contacting the target environment with at least one phosphorous boron-bonded compound of the disclosure selected from a phosphine-borane of Formula I, a cyclic phosphine-borane of Formula III and a phosphine-borane salt of Formula IV, can be performed in different ways which can be selected by a skilled person depending on the specific target environment where the carbon dioxide is captured.

Formation of the phosphino-borane-carbon dioxide complex Formula V can be detected with methods such as Nuclear Magnetic Resonance (NMR) Spectroscopy (e.g. ¹¹B NMR: ³¹P NMR and ¹H/¹³C NMR where the phosphino-borane-carbon dioxide complex of Formula V shows distinct upfield/downfield shifts), X-ray Crystallography (e.g. through direct visualization of the CO₂ binding in phosphino-borane-carbon dioxide complex Formula V) Infrared (IR) Spectroscopy (e.g. through different stretching modes of Free Co2 vs CO₂ capture in phosphino-borane-carbon dioxide complex Formula V) and UV-Vis Spectroscopy (e.g. through detection of charge transfer bands).

In some embodiments, of methods directed to capture carbon dioxide from a target environment, the method further comprises contacting the at least one phosphorous boron-bonded compound base is between 10 seconds and 1 hour. in particular when the at least one phosphorous boron-bonded compound comprises a compound of Formula I and/or Formula III.

In some embodiments, of methods directed to capture carbon dioxide from a target environment, the contacting time with CO₂ is between 30 seconds and 24 hours.

In embodiments herein described the contacting is performed in a reaction environment which can be the same or different from the target environment where the CO₂ is known or suspected to be present.

In some embodiments the target environment and/or the reaction environment is a liquid environment and the CO₂ is present or added to the liquid environment in combination with one or more phosphorous boron-bonded compounds of the disclosure and possibly in combination with one or more bases. In some of those embodiments the CO₂ is dissolved in a solvent, e.g. from a gaseous phase as will be understood by a skilled person upon reading of the present disclosure.

In some embodiments the target environment and/or the reaction environment is a gaseous phase and the CO₂ is present or added to the gaseous phase in combination with one or more phosphorous boron-bonded compounds of the disclosure and possibly in combination with one or more bases. In some of those embodiments the gaseous phase comprises a flowing stream of gas. In some of those embodiments the gaseous phase comprises a static mixture of gases.

In some of those embodiments and the CO₂ present or added in the flowing gas stream and/or the static mixture of gases is introduced from the gaseous stream to a in solvent which form part of a reaction environment.

In some embodiments the target environment and/or the reaction environment is a liquid and/or gas environment in combination with a solid support and in combination with one or more phosphorous boron-bonded compounds of the disclosure and possibly in combination with one or more bases. In some of those embodiments the one or more phosphorous boron-bonded compounds can be presented on the solid support. In some of those embodiments a reaction environment can comprise spaces within the solid support where the CO₂ is confined as will be understood by a skilled person upon reading of the disclosure.

In some embodiments, the contacting according to methods herein described to capture CO₂, is performed by direct gas-phase interaction. In those embodiments the contacting can be performed by exposing to gaseous CO₂, the at least one phosphorous boron-bonded compound of the disclosure selected from a phosphine-borane of Formula I, a cyclic phosphine-borane of Formula III and a phosphine-borane salt of Formula IV, for a time and under condition allowing capture of the gaseous CO₂. For example the exposing can be performed at ambient or elevated pressures until formation of a detectable amount of phosphino-borane-carbon dioxide complex Formula V, for example above a set threshold detected quantity of phosphino-borane-carbon dioxide complex Formula V.

In some embodiments, the contacting according to methods herein described to capture CO₂, is performed by solution phase interaction. In those embodiments, solvents (e.g., toluene, THF) can be used to dissolve phosphino-boranes and facilitate CO₂ activation the contacting can be performed by dissolving the CO₂, in the solvent and before concurrently or after adding in the solvent at least one phosphorous boron-bonded compound of the disclosure selected from a phosphine-borane of Formula I, a cyclic phosphine-borane of Formula III and a phosphine-borane salt of Formula IV. for a time and under condition allowing capture of the gaseous CO₂. For example until formation in the solvent of a detectable amount of phosphino-borane-carbon dioxide complex Formula V, for example above a set threshold detected quantity of phosphino-borane-carbon dioxide complex Formula V.

In embodiments of methods of the disclosure directed to the capture of CO2, a factor affecting carbon dioxide capture comprise solvent polarity: Solvation dynamics do play a role. Electron-donating solvents stabilize the product state, decreasing overall ΔG of CO2 binding by +3 kcal or so. Electron-poor solvents have no such effect. Suitable solvents typically have a dielectric constant ε≥7 preferably 7.5-80.

Accordingly in embodiments where the contacting of CO₂ with at least one phosphorous boron-bonded compound of the disclosure is performed in a liquid environment, the solvent is typically an aprotic solvent.

The term “aprotic solvent” as used herein indicates solvents that do not have hydrogen atoms attached to electronegative atoms like oxygen or nitrogen, and therefore cannot donate hydrogen bonds. In embodiments herein described, aprotic solvents and in particular polar aprotic or ethereal solvents are particularly suitable because they do not interfere with the Lewis acid-base interactions or react with sensitive P—B bonds, allowing for clean reactivity and stable handling of compounds of the disclosure as will be understood by a skilled person. These solvents are widely used in research and industry for reactions involving phosphine-borane compounds due to their ability to dissolve both neutral and ionic species, their lack of hydrogen-bond donation, and their generally inert behavior toward sensitive main-group bonds as will be understood by a skilled person.

Exemplary aprotic solvent suitable for reaction of phosphine-borane of Formula I, a cyclic phosphine-borane of Formula III and a phosphine-borane salt of Formula IV with CO₂ according to method of the disclosure comprise tetrahydrofuran (THF), diethyl ether, tert-Butyl methyl ether, 1,4-Dioxane, Dimethylformamide (DMF), Dimethyl sulfoxide (DMSO), N-Methyl-2-pyrrolidinone (NMP), Acetonitrile (CH₃CN), Acetone, Ethyl acetate, Sulfolane and N-Butyl-2-pyrrolidinone (NBP) and additional aprotic solvent identifiable by a skilled person upon reading of the present disclosure.

In embodiments of methods of the disclosure directed to the capture of CO2, a factor affecting carbon dioxide capture comprise temperature.

In embodiments of methods of the disclosure directed to the capture of CO2, a factors affecting carbon dioxide capture comprise cation effects: Suitable cation concentrations typically range from 0.01-0.1 equivalents to avoid side reactions.

In some embodiments, the contacting according to methods herein described to capture CO₂, is performed in immobilized systems. In those embodiments, at least one phosphorous boron-bonded compound of the disclosure selected from a phosphine-borane of Formula I, a cyclic phosphine-borane of Formula III and a phosphine-borane salt of Formula IV is anchored to a solid support which is then contacted with the target environment for a time and under conditions allowing formation of a compound of Formula V. For example the contacting of the solid support presenting at least one phosphorous boron-bonded compound of the disclosure can be performed at ambient or elevated pressures until formation of a detectable amount of phosphino-borane-carbon dioxide complex Formula V, for example above a set threshold detected quantity of phosphino-borane-carbon dioxide complex Formula V.

The term “support” as used herein indicates a solid material configured to provide a physical and often chemical platform for the immobilization, dispersion, or stabilization of active chemical species. The support enhances the accessibility, reactivity, and reusability of the active component by offering high surface area, tunable porosity, and mechanical or thermal stability, thereby enabling efficient interaction with target molecules in processes such as gas capture, catalysis, or separation as will be understood by a skilled person. Exemplary support suitable for methods and systems herein described comprise inorganic oxide, polymer, carbonaceous substance, or porous framework which can be configured to present phosphorous boron-bonded compound of the disclosure and/or provide confinement space and/or reaction environment for CO₂.

Exemplary solid support which can be used in connection with the methods and systems of the disclosure to capture CO₂ from a target environment comprise polymeric supports such as polymeric matrices which are continuous phase composed of long-chain macromolecules—polymers—that serves as the backbone or host material in composite systems, providing structural cohesion, shape, and environmental resistance to the overall material In this matrix, the polymer chains can be natural or synthetic and may be arranged in linear, branched, or crosslinked configurations, each influencing the mechanical and physical properties of the matrix as will be understood by a skilled person.

Further exemplary supports comprise silica-based supports (such as silica materials, including mesoporous silica, commonly used in catalysis and sorption science usually facilitating gas-solid interactions.

Additional exemplary supports comprise carbonaceous materials such as activated carbon and other carbon-based supports which can be functionalized to immobilize P phosphorous boron-bonded compounds of the disclosure and usually offer high surface area, chemical stability, and the potential for further modification to enhance selectivity or capacity for CO₂ capture).

Further exemplary supports comprise inorganic supports, such as zeolites, clays, and talc) which can be e modified or impregnated with one or more phosphorous boron-bonded compound of the disclosure to create hybrid sorbents with tailored properties for specific applications.

In some preferred embodiments the solid support is a sorbent. The term “sorbent” as used herein indicates a porous, high-surface-area material that serves as a structural scaffold to immobilize and disperse an active sorbent component. In methods and systems of the disclosure the active sorbent component can comprise one or more one or more phosphorous boron-bonded compound of the disclosure as will be understood by a skilled person. The support enhances the sorbent's stability, accessibility, and efficiency by providing a large contact area for gas-solid interactions while minimizing aggregation of active sites, as will be also understood by a skilled person.

In embodiments of methods and systems to capture CO₂ of the disclosure wherein the reaction environment comprise a solid support the solid support can be engineered to confine and/or present one or more phosphorous boron-bonded compound of the disclosure, the CO2 and/or one or more additional reactants (e.g. one or more base when one or more phosphorous boron-bonded compound have Formula I and/or III) to allow performance of the CO2 capture as described herein.

In some embodiments, methods and systems for CO₂ capture of the disclosure wherein a solid sorbent is used, the solid sorbent is a polymer material, a silica or alumina material, a zeolite or a metal-organic framework.

In some embodiments, methods and systems for CO₂ capture of the disclosure wherein a solid sorbent is used, the sorbent material is porous, preferably with pore sizes ranges between 1-500 nm.

In some embodiments, methods and systems for CO₂ capture of the disclosure wherein a solid sorbent is used, the sorbent material has a surface area from 0.1-100 m²/g.

In some preferred embodiments of the methods and system to capture carbon dioxide with one or more by phosphorous boron-bonded compound of the disclosure, the contacting can be performed by contacting the target environment with a material and/or device configured to create selective storage spaces for CO₂ after separation from the environment.

In some of those embodiments, the at least one phosphorous boron-bonded compound of the disclosure can be contacted with the CO₂ within the storage space of the material and/or device. In addition or in the alternative the at least one phosphorous boron-bonded compound of the disclosure can be contacted with the CO₂ following release of CO₂ the storage space of the material and/or device into a reaction environment where the at least one at least one phosphorous boron-bonded compound is presented for reaction with the CO₂.

In some embodiments the material and/or device comprise a compressed CO₂ injection systems configured to hold pressured CO₂ gas retrieved from a target environment which can be released from the target environment into a reaction environment comprising at least one phosphorous boron-bonded compound of the disclosure selected from a phosphine-borane of Formula I, a cyclic phosphine-borane of Formula III and a phosphine-borane salt of Formula IV presented for reaction with the CO₂.

Exemplary material and/or device within a compressed CO₂ injection systems comprise compressed CO₂ tanks (e.g. 20-50 lbs capacity, 1600-2200 PSI), Pressure regulators (e.g. configured to reduce pressure to 100-200 PSI), Flow meters (e.g., 20 CFH for precise delivery). and Solenoid valves and 24-hour timers (e.g., 2-minute pulses every 2 hours).

Exemplary target environments where CO₂ injection systems can be used comprise greenhouses or grow rooms (e.g., raising CO₂ from 200 ppm to 1500 ppm). and additional environment where precise control, no heat emission, scalable for small to medium spaces are desired.

In some embodiments, a material and/or device configured to create selective storage spaces for CO₂ comprise solid sorbents (such as adsorbents) which are configured to have selective space to capture CO₂ which can be used as a reaction environment according to methods and systems for CO₂ capture herein described. In those embodiments CO₂ can be captured in the solid sorbent and then reacted with at least one phosphorous boron-bonded compound of the disclosure within the selective space comprising the CO₂.

In addition or in the alternative CO₂ can be released in a reaction environment further comprising the at least one phosphorous boron-bonded compound of the disclosure presented for reaction with the CO₂.

An exemplary solid sorbent configured to have storage space for CO₂ comprise MOFs (Metal-Organic Frameworks) in which CO₂ can be separated via, physisorption via van der Waals forces or chemisorption with functional groups (e.g., —NH₂), and then released via heating or pressure swing. An exemplary MOFs is SiF6-3-Cu MOF which efficiently captures CO₂ at partial pressures <1%.

A further exemplary solid sorbent configured to have storage space for CO₂ comprise zeolites configured to hold CO₂ through electrostatic interactions in aluminosilicate cages, typically with thermal stability (>400° C.), and low cost Exemplary zelites comprise 13X which has CO₂ capacity of −2.5 mmol/g at 25° C., and 4A: which can perform selective adsorption via aluminum sites.

Exemplary devices comprising solid sorbent configured to have storage space for CO₂ comprise fixed-bed reactors, temperature/pressure-swing regeneration systems, and additional devices identifiable by a skilled person.

In some embodiments the material and/or device configured to create selective storage spaces for CO₂ after separation from the environment comprise Liquid Sorbents in Rotating Packed Beds (RPBs). In those embodiments amine-based solvents (e.g., monoethanolamine) in centrifugally enhanced reactors to absorb diluted CO₂.

The term “amine-based solvents” are liquid solutions-most commonly aqueous-containing organic amines that chemically react with carbon dioxide (CO₂) and other acid gases to selectively absorb and remove them from gas streams.

In some embodiments amine-based solvents operate in a cyclic process: they absorb CO₂ in an absorber column, and then release (desorb) it in a regenerator column upon heating, allowing the solvent to be reused. In particular in the absorber, the amine reacts with CO₂ to form soluble carbamate or bicarbonate species, depending on the amine type. The CO₂-rich solvent is then heated in a regenerator, releasing pure CO₂ and regenerating the amine solution for another cycle.

In some embodiments, amine-based solvents in an environment further comprising the at least one phosphorous boron-bonded compound of the disclosure presented for reaction with the CO₂.

Exemplary amine-based solvents comprise Monoethanolamine (MEA): Diethanolamine (DEA): Methyldiethanolamine (MDEA): Diisopropanolamine (DIPA) and Aminoethoxyethanol (Diglycolamine, DGA).

Exemplary devices comprising liquid sorbent configured to have storage space for CO₂ comprise RPBs (Rotating Packed Beds) with high turbulence for gas-liquid contact and Regeneration columns to release CO₂. Exemplary applications can reach an efficiency: >80% capture from streams with <5% CO₂ as will be understood by a skilled person.

Additional devices that can be used in connection with methods and systems for capturing CO2 comprise CO₂ Controllers and Sensors such as CO₂ meters (e.g., NDIR sensors) for real-time monitoring. Automated controllers (e.g., integrating timers, alarms, and solenoid valves). And Distribution systems (e.g., perforated PVC pipes in greenhouses) which can be used to Maintain optimal CO₂ levels (e.g., 550-1500 ppm) via feedback.

For very diluted sources (<2% CO₂), hybrid systems or RPBs are preferred, while compressed gas and generators suit controlled environments like greenhouses as will be understood by a skilled person.

In some embodiments, where the reaction environment comprises a solvent, at least one phosphorous boron-bonded compound can be added by dissolving in the solvent the at least one phosphorous boron-bonded compound to obtain a solution wherein the at least one phosphorous boron-bonded compound is dispersed within the solvent.

In some embodiments, where the reaction environment comprises a solvent which is an oil, at least one phosphorous boron-bonded compound can be added by dispersing in the oil the at least one phosphorous boron-bonded compound to obtain a dispersion wherein the at least one phosphorous boron-bonded compound is dispersed within the oil.

In some embodiments, where the reaction environment comprises a solid support such as a polymeric matrix at least one phosphorous boron-bonded compound can be added by contacting the at least one phosphorous boron-bonded compound with the solid matrix to obtain a modified support wherein the at least one phosphorous boron-bonded compound is incorporated on the solid support.

In some embodiments, the devices, supports, reactants comprising phosphorous-boron bonded compound and one or more base can be comprised in combination within a system for capturing carbon dioxide from a target environment, in a configuration allowing contacting of at least one phosphino-borane compound with the target environment known or suspected to include CO₂.

For example some embodiment, one or more phosphorous-boron bonded compound can be included in a bioreactor together with a target environment such as waste management environment, to which a bio reactive yeast, fungus and/or bacterium are also added which can convert the capture carbon dioxide into another product.

Additional combinations of devices and/or reagents can be configured depending on the target environment, and phosphorous boron-bonded compound of the disclosure as well as downstream reaction if any as will be understood by a skilled person.

In this connection, in embodiments of the disclosure the combination of reactants, devices, supports and equipment to capture of CO₂ by phosphorous boron-bonded compound of the disclosure can be selected based on the specific environment and compounds used as well as downstream reactions herein described as will be understood by a skilled person. Accordingly CO₂ capture by phosphorous boron-bonded compound of the disclosure can thus be performed with reactivity finely tunable via structural and environmental modulation as will be understood by a skilled person upon reading of the present disclosure.

In certain embodiments, the phosphine-borane of Formula I or Formula III is configured with appropriate substituents R₁ to R₅ that afford a P—H bond with pKa between 5 and 14 at 298K and 1 atm, as will be understood by the skilled person.

In other embodiments, the phosphine-borane of Formula I or Formula III is configured with appropriate substituents R₁ to R₅ that afford a P—H bond with pKa between 14 and 25 at 298K and 1 atm, as will be understood by the skilled person.

The skilled person will select appropriate substituents for the phosphine-borane of Formula I or Formula III first by considering the solvent required for the application. For example, if the solvent is water the pKa range for the P—H bond should be between 5 and 14 at 298K and 1 atm due to the solvent window of water. Similarly, if the solvent is THF, acetonitrile, dimethysulfoxide, N,N-dimethylformamide, hexane, toluene or an ionic liquid the pKa range for the P—H bond should be between 5 and 25 at 298K and 1 atm due to the wider solvent windows of these solvents.

The skilled person will appreciate that substituents R₁ to R₅ may be classified in terms of their electronic properties and how these effect the electron density of the P and/or B atoms to which they are attached. Electron-donating groups (EDGs) serve to increase electron density on the attached atoms, increasing their Lewis basicity. Alternatively, electron-withdrawing groups (EWGs) serve to decrease electron density on the attached atoms, increasing their Lewis acidity. Variation of the electronic density (and the subsequent Lewis basicity/Lewis acidity) of the P and/or B atoms will vary the physical properties of the phosphine-borane as described below.

EDGs include alkyl groups such as methyl, ethyl, propyl, butyl and cyclic and higher alkyl moieties; dialkylamino groups such as N(CH₃)₂ and N(C₂H₅)₂; alkoxy groups such as O—CH₃, O—C₂H₅; and guanidyl groups of Formula II such as —N(CH₃)—C═(N(CH₃)₂)—(N(CH₃)₂).

EWGs include groups such as fluorine; aryl groups such as phenyl and substituted phenyl; and fluoroalkyl groups such as CF₃.

The skilled person will understand that substitution at R₁ to R₅ with EDGs serves to increase the electron density on the P and/or B atom to which it is attached, increasing the pKa of the P—H bond of Formula I or Formula III. Likewise, substitution at R₁ to R₅ with EWGs serves to decrease the electron density on the P and/or B atom to which it is attached. For example, the phosphine-borane with EDGs R₁ and R₂=CH₃ and R₃, R₄ and R₅=H has pKa=23; conversely, the phosphine-borane with EWGs R₁ and R₂=Ph and R₃, R₄ and R₅=H has pKa=17.

The skilled person will also appreciate that choosing a substituent pattern whereby the phosphine-borane comprises part of the 5- or 6-membered ring of Formula III will result in a lower pKa compared to the acyclic structure. For example, the phosphine-borane where R₁ is a phenyl group, R₂ and R₃ are connected by a 4-carbon chain, and R₄ and R₅=H has pKa=16; similarly, the phosphine-borane where R₁ is a phenyl group, R₂ and R₃ are connected by a 3-carbon chain, and R₄ and R₅=H has pKa=12.

TABLE 1
variation of P—H bond pKa with substituent type

| | | | | | pKa of |
|---|---|---|---|---|---|
| | | | | | P—H |
| R₁ | R₂ | R₃ | R₄ | R₅ | bond |
| CH₃ (EDG) | CH₃ (EDG) | H | H | H | 23 |
| Ph (EWG) | Ph (EWG) | H | H | H | 17 |

| | | | | |
|---|---|---|---|---|
| Ph | connected by 4- | H | H | 16 |
| | carbon chain | | | |
| Ph | connected by 3- | H | H | 12 |
| | carbon chain | | | |

In certain embodiments, the phosphine-borane salt of Formula IV is configured with appropriate substituents R₁ to R₅ that afford a reactivity toward CO₂ with a ΔG value 0<ΔG<−5 kcal/mol at 298K and 1 atm, enabling capture of CO₂ by the phosphine-borane salt to form the phosphinoborane-carbon dioxide complex of Formula V in a reversible reaction.

In other embodiments, the phosphine-borane salt of Formula IV is configured with appropriate substituents R₁ to R₅ that afford a reactivity toward CO₂ with a ΔG value −5<ΔG kcal/mol at 298K and 1 atm, enabling capture of CO₂ by the phosphine-borane salt to form the phosphinoborane-carbon dioxide complex of Formula V in an irreversible reaction.

The skilled person will select appropriate substituents for the phosphine-borane salt of Formula IV first by considering whether the application of the resulting phosphinoborane-carbon dioxide complex requires reversible or irreversible binding of CO₂. For example, use of the phosphinoborane-carbon dioxide complex of Formula V as a chemical feedstock will require this complex to exhibit reversible binding of CO₂ and, hence, the phosphine-borane salt of Formula IV needs to be configured with appropriate substituents R₁ to R₅ that afford a reactivity toward CO₂ with a ΔG value 0<ΔG<−5 kcal/mol at 298K and 1 atm. Alternatively, use of the phosphinoborane-carbon dioxide complex of Formula V in CO₂ sequestration will require this complex to exhibit irreversible binding of CO₂ and, hence, the phosphine-borane salt of Formula IV needs to be configured with appropriate substituents R₁ to R₅ that afford a reactivity toward CO₂ with a ΔG value −5<ΔG kcal/mol at 298K and 1 atm.

The skilled person will appreciate that substituents R₁ to R₅ may be classified in terms of their electronic properties as EDGs or EWGs as above. Here, EDGs serve to increase electron density on the attached atoms, for example increasing the nucleophilicity of the phosphorous atom in the phosphine-borane salt of Formula IV and increasing the strength of its binding to CO₂; similarly EDGs on the boron atom serve to increase the stabilization of the bound CO₂ moiety, increasing the overall CO₂ binding strength of the phosphine-borane salt of Formula IV. The reverse is true for EWGs, decreasing the CO₂ binding strength if substituted on the phosphorous or fboron.

The skilled person will also appreciate that choosing a substituent pattern whereby the phosphine-borane salt comprises part of the 5- or 6-membered ring of Formula III will result in a higher CO₂ binding strength compared to the acyclic structure, as the P and B atoms are held tightly in a preferred binding geometry due to the rigid ring structure, resulting in a stronger-bound CO₂ complex.

The CO₂ binding strength of the phosphine-borane salt of Formula IV may be further tuned by selecting the counterion Mnⁿ⁺ to either decrease or increase <ΔG value for reactivity toward CO₂ as desired. The skilled person will understand that a small and/or highly-charged ion such as Li⁺, Na⁺, Mg²⁺ or Al³⁺ has a high charge density and will form a tight ion pair with the anionic phosphine-borane species in Formula IV; this results in a secondary interaction between Mnⁿ⁺ and the CO₂ moiety in the phosphinoborane-carbon dioxide complex of Formula V, stabilizing this structure and increasing the increasing the CO₂ binding strength. Conversely, a larger ion such as K⁺, Rb⁺, Cs⁺ or tetraalkylammonium will have a lower charge density and will form a more loosely-bound ion pair with the anionic phosphine-borane species in Formula IV; in these cases the phosphinoborane-carbon dioxide complex of Formula V is less stable and the CO₂ binding strength is consequently lower.

Additionally, the skilled person will select the counterion Mn⁺ in the phosphine-borane salt of Formula IV to ensure solubility of this salt and the resulting phosphinoborane-carbon dioxide complex of Formula V in the solvent of choice for a particular application at the appropriate concentration. For example, Li⁺ salts will be understood by the skilled person to be highly soluble in polar aprotic solvents such as THF, acetonitrile, dimethyl sulfoxide and N,N-dimethylformamide, up to 2M concentrations. Likewise, salts of larger organic-substituted cations such as tetraalkylammonium ions will be understood by the skilled person to be soluble in less polar solvents such as hexane or toluene, up to 0.5M concentrations.

Effects of variation of the substituent configuration with EWG and EDG on reactivity toward CO₂ in the phosphine-borane salt of Formula IV with a fixed counterion (Li⁺) are given below:

For M=Li, R₁ and R₂=CH₃ (EDG) and R₃, R₄ and R₅=H the ΔG value of CO₂ binding to the P atom is around −23.1 kcal/mol at 298K and 1 atm.

For M=Li, R₁ and R₂=C₂H₅(EDG) and R₃, R₄ and R₅=H the ΔG value of CO₂ binding to the P atom is around −24.0 kcal/mol at 298K and 1 atm.

For M=Li, R₁ and R₂=C₄H₉(EDG) and R₃, R₄ and R₅=H the ΔG value of CO₂ binding to the P atom is around −19.0 kcal/mol at 298K and 1 atm.

For M=Li, R₁ and R₂=N(CH₃)₂(EDG) and R₃, R₄ and R₅=H the ΔG value of CO₂ binding to the P atom is around −14.3 kcal/mol at 298K and 1 atm.

For M=Li, R₁ and R₂=OCH₃ (EDG) and R₃, R₄ and R₅=H the ΔG value of CO₂ binding to the P atom is around −7.6 kcal/mol at 298K and 1 atm.

For M=Li, R₁ and R₂=—N(CH₃)—C═(NCH₃)—N(CH₃)₂(EDG) and R₃, R₄ and R₅=H the ΔG value of CO₂ binding to the P atom is around −7.4 kcal/mol at 298K and 1 atm.

For M=Li, R₁ and R₂=F (EWG) and R₃, R₄ and R₅=H, the ΔG value of CO₂ binding to the P atom is around −1.3 kcal/mol at 298K and 1 atm.

For M=Li, R₁ and R₂=C₆F₅(EWG) and R₃, R₄ and R₅=H, the ΔG value of CO₂ binding to the P atom is around −0.4 kcal/mol at 298K and 1 atm.

For M=Li, R₁ and R₂=CH₃ (EDG) and R₃, R₄ and R₅=CH₃ (EDG), the ΔG value of CO₂ binding to the P atom is around −22.3 kcal/mol at 298K and 1 atm.

For M=Li, R₁ and R₂=CF₃ (EWG) and R₃, R₄ and R₅=CF₃ (EWG), the ΔG value of CO₂ binding to the P atom is around +0.8 kcal/mol at 298K and 1 atm, and hence is thermodynamically-unfavorable.

TABLE 2
variation of CO₂ binding strength with substituent type

| | | | | | | ΔG for |
|---|---|---|---|---|---|---|
| Mⁿ⁺ | R₁ | R₂ | R₃ | R₄ | R₅ | CO₂ binding |
| Li | CH₃ (EDG) | CH₃ (EDG) | H | H | H | −23.1 |
| Li | C₂H₅ (EDG) | C₂H₅ (EDG) | H | H | H | −24.0 |
| Li | C₄H₉ (EDG) | C₄H₉ (EDG) | H | H | H | −19.0 |
| Li | N(CH₃)₂ (EDG) | N(CH₃)₂ (EDG) | H | H | H | −14.3 |
| Li | OCH₃ (EDG) | OCH₃ (EDG) | H | H | H | −7.6 |
| Li | —N(CH₃)—C═(NCH₃)—N(CH₃)₂ | —N(CH₃)—C═(NCH₃)—N(CH₃)₂ | H | H | H | −7.4 |
| | (EDG) | (EDG) | | | | |
| Li | F (EWG) | F (EWG) | H | H | H | −1.3 |
| Li | C₆F₅ (EWG) | C₆F₅ (EWG) | H | H | H | −0.4 |
| Li | CH₃ (EDG) | CH₃ (EDG) | CH₃ (EDG) | CH₃ (EDG) | CH₃ (EDG) | −22.3 |
| Li | C₆F₅ (EWG) | C₆F₅ (EWG) | C₆F₅ (EDG) | C₆F₅ (EDG) | C₆F₅ (EDG) | +0.8 |

The effect of the phosphine-borane salt comprising a cyclic P—B ring structure is evident from the comparisons below:

For M=Li, R₁ and R₂=Ph and R₃, R₄ and R₅=H, the ΔG value of CO₂ binding to the P atom is around −12.8 kcal/mol at 298K and 1 atm.

For M=Li, R₁=Ph, R₂ and R₃ are connected by a 4-carbon chain and R₄ and R₅=H, the ΔG value of CO₂ binding to the P atom is around −13.4 kcal/mol at 298K and 1 atm.

For M=Li, R₁=Ph, R₂ and R₃ are connected by a 3-carbon chain and R₄ and R₅=H, the ΔG value of CO₂ binding to the P atom is around −20.2 kcal/mol at 298K and 1 atm.

TABLE 3
variation of CO₂ binding strength
when comprising a cyclic P—B ring

| | | | | | | ΔG for |
|---|---|---|---|---|---|---|
| Mⁿ⁺ | R₁ | R₂ | R₃ | R₄ | R₅ | CO₂ binding |
| Li | Ph | Ph | H | H | H | −12.8 |

| | | | | | |
|---|---|---|---|---|---|
| Li | Ph | connected by 4- carbon chain | H | H | −13.4 |
| Li | Ph | connected by 3- carbon chain | H | H | −20.2 |

Effects of variation of the counterion Mⁿ⁺ on reactivity toward CO₂ in the phosphine-borane salt of Formula IV with a fixed substituent pattern are given below:

For M=Li, R₁ and R₂=CH₃ and R₃, R₄ and R₅=H the ΔG value of CO₂ binding to the P atom is around −23.1 kcal/mol at 298K and 1 atm.

For M=Na, R₁ and R₂=CH₃ and R₃, R₄ and R₅=H the ΔG value of CO₂ binding to the P atom is around −19.8 kcal/mol at 298K and 1 atm.

For M=K, R₁ and R₂=CH₃ and R₃, R₄ and R₅=H the ΔG value of CO₂ binding to the P atom is around −17.9 kcal/mol at 298K and 1 atm.

For M=Li, R₁ and R₂=Ph and R₃, R₄ and R₅=H the ΔG value of CO₂ binding to the P atom is around −12.8 kcal/mol at 298K and 1 atm.

For M=Na, R₁ and R₂=Ph and R₃, R₄ and R₅=H the ΔG value of CO₂ binding to the P atom is around −8.8 kcal/mol at 298K and 1 atm.

For M=K, R₁ and R₂=Ph and R₃, R₄ and R₅=H the ΔG value of CO₂ binding to the P atom is around −4.9 kcal/mol at 298K and 1 atm.

TABLE 4
variation of CO₂ binding strength with Mⁿ⁺ counterion type

| | | | | | | ΔG for |
|---|---|---|---|---|---|---|
| Mⁿ⁺ | R₁ | R₂ | R₃ | R₄ | R₅ | CO₂ binding |
| Li | CH₃ | CH₃ | H | H | H | −23.1 |
| Na | CH₃ | CH₃ | H | H | H | −19.8 |
| K | CH₃ | CH₃ | H | H | H | −17.9 |
| Li | Ph | Ph | H | H | H | −12.8 |
| Na | Ph | Ph | H | H | H | −8.8 |
| K | Ph | Ph | H | H | H | −4.9 |

CO₂ captured according to methods and systems of the disclosure can be further used according to desired CO₂ processing cycles.

In particular methods and systems for capturing CO₂ herein described can be used for CO₂ Capture/Storage, in connection with CO₂ reversible systems (e.g., for selective gas separation) for chemical synthesis involving CO₂ (e.g. for CO₂ conversion to formates, methanol, or carbamates and/or for catalysis: (e.g. for metal-free reduction of CO₂ using hydroboranes or silane) as will be understood by a skilled person.

A skilled person will select a species of formula IV that has a highly-negative ΔG for CO₂ binding (ΔG<−5 kcal/mol) to irreversibly bind CO₂, for example to scrub CO₂ from a waste stream or from the air. Subsequent to binding and formation of a complex of formula V this complex (either as a solid or in solution) will be transferred to a storage unit and sealed (for example, buried underground or sealed inside a mountain or waste dump), thus sequestering the CO₂ permanently.

A skilled person will select a species of formula IV that has a ΔG for CO₂ binding less than zero but not greater than −5 kcal/mol (0<ΔG≤−5 kcal/mol) to reversibly bind CO₂, for example to concentrate CO₂ from a waste stream or from the air for subsequent use as a CO₂-source for chemical reactions. Subsequent to binding and formation of a complex of formula V this complex (either as a solid or in solution) will be transferred to a reactor unit (for example, a bioreactor or a chemical reactor) and subjected to further chemical reactions, thus serving as a means to capture the CO₂ in one set of conditions and release it for further use under a different set of conditions.

The skilled person will understand that complexes of Formula V configured for “reversible” binding of CO₂ may undergo a series of reactions whereby moieties are transferred to the CO₂ and/or the phosphorous or boron atoms, resulting in a catalytic cycle that effectively releases a CO₂-containing product (e.g., a reduction product) while regenerating the precursor species of Formula IV in a catalytic cycle. For example, formate may be generated from the addition of hydride (H⁻) to bound CO₂ via a metal hydride reagent that may itself be re-generated from hydrogen according to the following scheme:

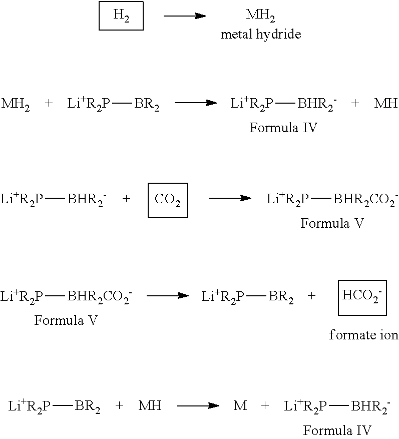

where the skilled person will select the R groups appropriately so that 0<ΔG≤−5 kcal/mol for CO₂ binding to the species of Formula IV, for example R₁ and R₂=F and R₃, R₄ and R₅=H, the ΔG value of CO₂ binding to the P atom is around −1.3 kcal/mol at 298K and 1 atm. Alternatively, CO₂ can be released from complexes of Formula V by thermal means, for example by heating the complex to 80° C., or 100° C., or 120° C., whereby the species of Formula IV is recovered after releasing CO₂ back into the environment.

As another alternative, CO₂ can be released from complexes of Formula V under vacuo, for example by subjecting a mixture containing a complex of Formula V to less-than-atmospheric pressure at 0.5 atm, or 0.1 atm, or 0.01 atm or 0.001 atm, whereby the species of Formula IV is recovered after releasing CO₂ back into the environment.

In some embodiments herein described, the methods and systems for capturing CO₂ can be used for chemical synthesis and thus to convert the captured CO₂ into one or more of downstream reaction products will be understood by a skilled person upon reading of the disclosure.

In particular, in some embodiments, a method is described for converting carbon dioxide from a target environment to a carbon dioxide reaction product. In those embodiments, following capturing of the carbon dioxide to provide the phosphorous boron-bonded dioxide complex of Formula V, the method further comprises reacting the compound of Formula V with a reducing agent and/or an alkylating agent to obtain one or more carbon dioxide reaction product of Formula VI

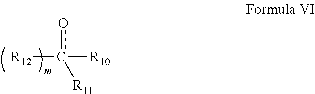

wherein
m is 0 or 1;
R₁₀, =H, OH, O-alkyl, O-aryl, CH₃, C(OH)H₂, C(O)H or C(O)OH
R₁₁ and R₁₂ are each independently H, OH, O-alkyl or O-aryl and
   wherein when n=0 and R₁₀=OH, R₁₁ is H, O-alkyl, O-aryl

The term “reducing agent” (also known as a reductant, reducer, or electron donor) as used herein indicates a chemical species that donates electrons to another substance in a redox (oxidation-reduction) reaction, thereby reducing that substance while itself becoming oxidized. The reducing agent loses electrons, causing its own oxidation state to increase, and in doing so, enables the reduction of another reactant whose oxidation state decreases. Common examples of reducing agents include hydrogen gas, carbon monoxide, alkali metals such as sodium and lithium, metal hydrides like lithium aluminum hydride (LiAlH₄) and sodium borohydride (NaBH₄), as well as organic compounds such as formic acid, oxalic acid, and ascorbic acid (vitamin C). Additional reducing agents such as ferrous sulfate, tin(II) chloride, and thiosulfates are also widely used. The strength of a reducing agent depends on its tendency to lose electrons easily, which is often associated with low electronegativity and low ionization energy as will be understood by a skilled person. A negatively-polarized electrode (a cathode in an electrolysis reaction) serves as a source of electrons during the reaction, so can also be considered as a reducing agent for the purposes of this disclosure.

The skilled person will select the reducing agent according to the reaction conditions available and products required. For example, if formate is the desired product, the skilled person can select a single hydride source such as a metal hydride or borohydride as outlined in the catalytic reaction scheme above.

Alternatively, if methanol is the desired product, the skilled person can select a source of multiple hydrides such as a metal aluminum hydride, or can choose to perform an electrochemical reduction on a suitable metal electrode such as platinum or copper, configured to transfer multiple electrons and/or H atoms.

If a 2- or more-carbon product is desired such as ethanol or acetic acid, a reductive coupling reaction between species of formula V can be performed on a suitable electrode such as copper, optionally in the presence of additional hydride and/or proton sources such as HCl, under conditions configured to favor reductive carbon-carbon and carbon-hydrogen bond formation is favored.

The term “alkylating agent” as used herein indicate an electrophilic reagents that transfer alkyl groups to an oxygen site or a carbon site of another compound, enabling the synthesis and functionalization of the compound. The most common alkylating agents used in this context are alkyl halides such as methyl iodide, benzyl bromide, 1-iodohexane, and 2-iodopropane, alkyl triflates such as methyl triflate and ethyl triflate, and alkyl sulfates such as dimethyl sulfate.

The skilled person will select the alkylating agent based on the reaction conditions available and the product desired. For example, if a methyl ether, ester or carbonate is desired a source of methyl group such as methyl iodide or methyl triflate will be selected. Similarly, for a ethyl ether, ester or carbonate a source of ethyl group such as ethyl bromide or diethyl sulfate will be chosen. For an alkylated polymer such as a polycarbonate product, the skilled person will select a di- or poly-functional alkylating agent such as 1,2-diiodoethane.

In some embodiments, a carbon dioxide reaction product of Formula VI can be provided by reacting the compound of Formula V with a reducing agent and/or an alkylating agent to obtain the carbon dioxide reaction product of Formula VI.

For example in some embodiments, Mel was used as an additional alkylating agent experimentally in the alkylation reported in the Example section. In some embodiments, computationally, ½H2, NaH, NaBH4, HCl, H2, and ½H2+e− (electrochemical additions) were used as a reductant source as will be understood by a skilled person upon reading of the disclosure,

some embodiments, a carbon dioxide reaction product of Formula VI can be provided by reacting the compound of Formula V with an alkylating agent is an alkyl halide, alkyl triflate or alkyl sulfonate, and m=0, R₁₀=O-alkyl and R₁=O-alkyl

In some embodiments, a carbon dioxide reaction product of Formula VI can be provided by reacting the compound of Formula V with the alkylating agent is an alkyl halide, alkyl triflate or alkyl sulfonate, and m=0, R₁₀=O-alkyl and R₁=O-alkyl such that the product of Formula VI is a polymer with the polymer chain joined through R₁₀ and R₁₁.

In some embodiments, of the method for converting carbon dioxide from a target environment to a carbon dioxide reaction product, the phosphino-borane is within a composition further comprising a solvent and contacting is performed within the composition.

In some embodiments, of the method for converting carbon dioxide from a target environment to a carbon dioxide reaction product, method further comprises dissolving the phosphino-borane in the solvent to provide the phosphino-borane composition.

In some embodiments, of the method for converting carbon dioxide from a target environment to a carbon dioxide reaction product, the contacting is performed on a solid sorbent having a surface configured for contacting a target environment, the solid sorbent presenting the phosphino-borane absorbed on the surface., the contacting is performed on the surface of the solid sorbent presenting the phosphino-borane absorbed on the surface.

In some embodiments, of the method for converting carbon dioxide from a target environment to a carbon dioxide reaction product, the contacting is followed or preceded by contacting the solid sorbent presenting the presenting the phosphino-borane absorbed on the surface with the base.

In some embodiments, of the method for converting carbon dioxide from a target environment to a carbon dioxide reaction product, the contacting is performed by exposing the phosphino-borane to a gaseous stream from the target environment

In some embodiments, of the method for converting carbon dioxide from a target environment to a carbon dioxide reaction product, the reacting comprises exposing the compound of Formula V to hydrogen gas, hydrogen chloride or a metal hydride or borohydride reducing agent

In some embodiments, of the method for converting carbon dioxide from a target environment to a carbon dioxide reaction product, the reacting comprises exposing the compound of Formula V to a metal hydride selected from one or more of lithium hydride, sodium hydride, potassium hydride and calcium hydride.

In some embodiments, of the method for converting carbon dioxide from a target environment to a carbon dioxide reaction product, the reacting comprises exposing the compound of Formula V to a metal hydride selected from one or more of lithium aluminum hydride and sodium borohydride.

In some embodiments, of the method for converting carbon dioxide from a target environment to a carbon dioxide reaction product, the method further comprises exposure of a compound of Formula V in a solvent to an electrode that generate as a reducing agent on the solvent or support for electrochemical reduction.

In some embodiments, of the method for converting carbon dioxide from a target environment to a carbon dioxide reaction product obtained by electrochemical reduction with an electrode comprising platinum, copper or an alloy thereof.

In some embodiments, of the method for converting carbon dioxide from a target environment to a carbon dioxide reaction product, the method further comprises exposing a compound of Formula (V) to an alkylating agent in a solvent.

In some embodiments, of the method for converting carbon dioxide from a target environment to a carbon dioxide reaction product, the alkylating agent is an alkyl halide, an alkyl triflate or an alkyl sulfonate.

In some embodiments, of the method for converting carbon dioxide from a target environment to a carbon dioxide reaction product, exposure to the alkylating agent is performed to obtain a carbonate ester or a polycarbonate

In some preferred embodiments, of the method for converting carbon dioxide from a target environment to a carbon dioxide reaction product, the reaction product is or comprises formic acid.

In some preferred embodiments, of the method for converting carbon dioxide from a target environment to a carbon dioxide reaction product, the reaction product is or comprises methanol.

In some preferred embodiments, of the method for converting carbon dioxide from a target environment to a carbon dioxide reaction product, the reaction product is or comprises acetic acid.

In some preferred embodiments, of the method for converting carbon dioxide from a target environment to a carbon dioxide reaction product, the reaction product is or comprises ethanol.

In some embodiments, of the method for converting carbon dioxide from a target environment to a carbon dioxide reaction product, the reaction product has Formula VI in which m=0, R₁₀=OH and R₁₁=H, the reaction product is formic acid, the reducing agent is hydrogen, hydrogen chloride or a metal hydride or borohydride.

In some embodiments, of the method for converting carbon dioxide from a target environment to a carbon dioxide reaction product, the reaction product has Formula VI in which m=0, R₁₀=H and R₁₁=H, the reducing agent is hydrogen, hydrogen chloride or a metal hydride or borohydride,

In some embodiments, of the method for converting carbon dioxide from a target environment to a carbon dioxide reaction product, the reaction product has Formula VI in which m=1, R₁₀, R₁₁ and R₁₂=H, the reaction product is methanol and where the reducing agent is hydrogen, hydrogen chloride or a metal hydride or borohydride.

In some embodiments, of the method for converting carbon dioxide from a target environment to a carbon dioxide reaction product, the reaction product has Formula VI in which m=0, R₁₀=OH and R₁₁=H the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=H and R₁₁=H the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀, R₁₁ and R₁₂=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=C(O)OH and R₁=OH and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=C(O)H and R₁=OH and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=C(O)H and R₁₁=H, and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=C(OH)H₂ and R₁₁=OH and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=C(OH)H₂ and R₁₁=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=CH₃ and R₁₁=OH, the reaction product is acetic acid and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=0, R₁₀=CH₃ and R₁₁=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀=C(O)OH, R₁₁=OH and R₁₂=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀=C(O)H, R₁₁=OH and R₁₂=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀=C(OH)₂H, R₁₁=OH and R₁₂=H, and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀=CH₃, R₁₁=OH and R₁₂=H and the reducing agent is obtained through an electrode such as platinum electrodes, copper electrodes or electrodes formed by an alloy thereof.

In some embodiments, of the method for converting carbon dioxide from a target environment to a carbon dioxide reaction product, the reaction product has Formula VI in which m=1, R₁₀=CH₃ and R₁₁ and R₁₂=H, the reaction product is ethanol and the reducing agent is obtained through an electrode such as platinum electrodes, copper electrodes or electrodes formed by an alloy thereof.

In some preferred embodiments, of the method for converting carbon dioxide from a target environment to a carbon dioxide reaction product, the reaction product is obtained by further reduction of the initial product of Formula VI to provide methane as the final product.

Selection of the proper phosphorous boron-bonded compounds for producing the proper compound of encompassed by Formula V, and possibly which can then in turn be converted to a downstream product of Formula VI can be aided by computational methods

In some embodiments, the configuration and features phosphorous boron-bonded compounds of the disclosure can be selected in view of a desired use by a computer implemented method for selecting a substituent configuration of a phosphorous boron-bond compound of the disclosure, the substituent configuration having energy change (ΔGₛₑₜ) equal to or lower than a set free energy change (ΔGₛₑₜ) for carbon dioxide binding.

The computer implemented method comprises modeling structures of the phosphorous boron-bond compound with varying substituents and counterions in a simulated environment, to obtain modeled structures of the phosphorous boron-bond compound in the simulated environment.

The computer implemented method further comprises modeling a plurality of structures of the phosphorous boron-bond compound, the plurality of structures comprising different substituents attached to the phosphorus atom, said substituents including electron-donating groups and electron-withdrawing groups.

The computer implemented method also comprises modeling structures of the phosphorous boron-bond compound in presence or absence of one or more counterion obtaining a plurality of modeled structures of the phosphorous boron-bond compound.

The computer implemented method additionally comprises calculating a free energy change (ΔG) for carbon dioxide binding for each modeled structure of the plurality of modeled structures by determining the free energies of the respective lowest-energy reactant and product conformers, to obtain calculated free energy change (ΔG) of carbon dioxide binding of the modeled structures of the phosphorous boron-bond compound.

The computer implemented method further comprising selecting a substituent configuration and counterions having a calculated free energy changes (ΔG) equal or to or lower than the set free energy change (ΔGₛₑₜ). The transition state was also modeled for the BoPh with —Ph groups, indicating that the rate of CO₂ binding is very fast with a low barrier. (+2.4 kcal/mol with Li+ in the system, and +4.4 kcal/mol with Na+ in the system.)

In some embodiments of the computer implemented method, modeling structures of the phosphorous boron-bond compound can be performed by performing Density Functional Theory (DFT) calculations using a defined functional (e.g., M06-2X with D3 correction) and basis set (e.g., 6-311G**++) (e.g., using a Polarizable Continuum Model for THF) and optimizing geometry of reactant conformers.

In some embodiments of the computer implemented method of the disclosure optimizing geometry of reactant conformers can be performed by
varying the position of the counterion of the modeled structures of the phosphorous boron-bond compound in the simulated environment and
selecting a lowest-energy conformation of the phosphorous boron-bond compound in the simulated environment
confirming a selected lowest energy conformation of the phosphorous boron-bond compound in the simulated environment as true minima and
calculating thermochemical properties of the selected lowest energy conformation of the phosphorous boron-bond compound in the simulated environment, including free energies (G) at a defined temperature (e.g., 298.15 K), using vibrational frequency calculations, optionally applying corrections for solvent effects.

In some embodiments of the computer implemented method of the disclosure modeling structures of the phosphorous boron-bond compound in presence or absence of one or more counterion can be performed with one or more counterion selected from Li⁺, Na⁺, K⁺, and optionally in the absence of a counterion to evaluate counterion effects.

In some embodiments of the computer implemented method of the disclosure, selecting a substituent configuration and counterions is performed evaluation, the transition state barriers (ΔG‡) for carbon dioxide binding. In particular the transition state was modeled via a relaxed coordinate scan, which found the highest energy between the product and reactant states, and the various distances associated between the CO2 and reactant. A transition state search was then used to identify the transition state, with one negative eigenvalue computed along the Hessian for full optimization/confirmation of such a saddlepoint in the transition state as will be understood by a skilled person upon reading of the present disclosure

In some embodiments of the computer implemented method of the disclosure evaluating the effect of counterion position on the calculated free energy change (ΔG) of carbon dioxide binding of the modeled structures of the phosphorous boron-bond compound, can be performed by
modeling different placements of the counterion relative to the phosphido-borane anion of the modeled structures and bound carbon dioxide
analyzing calculated properties, including atomic charges, changes in charge upon binding (ΔQ), bond angles, and bond orders, to correlate structural features and substituent types with the calculated free energy change (ΔG) of carbon dioxide binding. and
optionally calculating transition state barriers (ΔG‡) for carbon dioxide binding for selected structures to evaluate kinetic factors, including the influence of the counterion on said barriers.

The computer implemented method described herein can be implemented on a variety of computing hardware configurations. In one embodiment, the method can be executed on a high-performance workstation comprising at least one multi-core processor, such as an x86_64 compatible processor or an Apple M-series processor. Sufficient system memory (RAM) is provisioned, preferably at a minimum ratio of 4 GB per processor core, although requirements can vary depending on the specific system size and calculation parameters. Storage includes sufficient disk space for software installation (e.g., approximately 18 GB for core software, potentially more for databases) and substantial scratch disk space (e.g., a minimum of 60 GB, potentially much more) for temporary files and calculation results generated during execution. Faster local disk access, such as that provided by solid-state drives (SSD) or high-RPM hard disk drives, is advantageous for calculations involving significant data input/output operations. For visualization tasks associated with the method, a graphics card supporting hardware-accelerated OpenGL with dedicated onboard memory (e.g., at least 1 GB) and up-to-date vendor drivers is recommended.

In some embodiments the computer implemented method of the disclosure, for computationally intensive or large-scale calculations, the method can be implemented on a distributed computing cluster managed by a queueing system (e.g., Slurm, PBS Pro, LSF, Grid Engine). Such a cluster typically comprises multiple compute nodes interconnected via a high-speed network. Each node possesses one or more multi-core processors and significant system memory, potentially ranging from 8 GB to 16 GB or more per core for parallel calculations. The cluster architecture includes shared storage solutions (e.g., high-performance network-attached storage or parallel file systems like FSx for Lustre) accessible by all compute nodes to facilitate data sharing and reduce network bottlenecks to external file servers. Specific compute nodes can optionally be equipped with General-Purpose Graphics Processing Units (GPGPUs), such as NVIDIA Tesla, Quadro, or RTX series cards, compatible with the computational software (e.g., Jaguar) and relevant CUDA versions, to accelerate specific portions of the calculations where GPU parallelism is supported and beneficial. The use of high-bandwidth, low-latency interconnects (e.g., AWS Elastic Fabric Adapter) between nodes can further enhance performance for communication-intensive parallel tasks.

In some embodiments one or more phosphorous boron-bonded composition comprising at least one phosphorous-boron bonded compound selected from a phosphine-borane of Formula I, a cyclic phosphine-borane of Formula III and a phosphine-borane salt of Formula IV, together with at least one suitable vehicle.

In the context of the present disclosure, the term “vehicle” refers to any suitable carrier, excipient, diluent, solvent, or medium that is substantially inert with respect to the active phosphorus-boron bonded compounds and facilitates the storage, delivery, application, or use of the composition. The vehicle can be liquid, solid, or semi-solid and should be compatible with the active compound(s) and the intended application, such as, but not limited to, carbon dioxide capture. The vehicle serves to contain, dissolve, suspend, disperse, or otherwise incorporate the active compound(s), potentially aiding in their stability, handling, or interaction with the target substance (e.g., CO₂).

Exemplary suitable vehicles comprise: organic solvents, particularly aprotic organic solvents in which the phosphorus-boron bonded compounds and their reaction products (e.g., CO₂ adducts) exhibit solubility and stability. Examples organic solvent comprise ethers, such as tetrahydrofuran (THF), diethyl ether, dioxane, dimethoxyethane (DME), aromatic hydrocarbons, such as toluene or xylene, aliphatic hydrocarbons, such as hexane or heptane, chlorinated solvents such as dichloromethane or chloroform, and other polar aprotic solvents like acetonitrile, dimethylformamide (DMF), or dimethyl sulfoxide (DMSO), provided they demonstrate compatibility.

Exemplary suitable vehicles further comprise ionic liquids in particular in view of solubility properties and low vapor pressure which can impact the properties of the phosphorus-boron bonded compound.

Exemplary suitable vehicles also comprise solid supports. The active phosphorus-boron bonded compounds can be immobilized or adsorbed onto a solid support material, which would then act as the vehicle. Examples of supports might include porous materials like silica gels, zeolites, metal-organic frameworks (MOFs), or functionalized polymers, although compatibility and reactivity would need careful consideration.

Exemplary suitable vehicles additionally comprise polymeric matrices configured to incorporate the phosphorous boron bonded compounds within a suitable polymer matrix, either dissolved or dispersed, particularly for applications requiring a solid-phase capture medium.

In embodiments herein described, the choice of vehicle can depend on factors such as the specific phosphorus-boron bonded compound used, the desired concentration, the operating conditions (temperature, pressure), the method of CO₂ contact (e.g., bubbling through a solution, flowing over a solid), and requirements for potential regeneration or subsequent conversion steps.

In some embodiments of the present disclosure, the at least phosphorous boron-bonded compound is within a phosphorous boron-bonded composition further comprising a vehicle and the contacting is performed within the composition.

In some embodiments, the solvent can tetrahydrofuran or acetonitrile, N,N-dimethylformamide or dimethyl sulfoxide, hexane or toluene or an ionic liquid as will be understood by a skilled person

In some embodiments, the concentration of the phosphine-borane is between 2M and 0.001 M in the solvent

In some embodiments, the solvent is a mixture of two or more liquids such as mixture of an ionic liquid and tetrahydrofuran or an ionic liquid and acetonitrile.

In some embodiments, the phosphino-borane composition has a concentration from 1M to 0.001M, from 0.5M to 0.001

In some embodiments, in the phosphorous boron-bonded compound M is Li, R₁ and R₂ are phenyl groups, R₃, R₄ and R₅ are each H and the concentration of the phosphino-borane composition is 0.5M and 0.001M

In some embodiments, in the phosphorous boron-bonded compound M is Li, R₁ and R₂ are methyl groups, R₃, R₄ and R₅ are each H and the concentration of the phosphino-borane composition is 0.5M and 0.001M

In some embodiments, in the phosphorous boron-bonded compound M is Li, R₁ and R₂ are ethyl groups, R₃, R₄ and R₅ are each H and the concentration of the phosphino-borane composition is 0.5M and 0.001M

In some embodiments, in the phosphorous boron-bonded compound M is Li, R₁ and R₂ are butyl groups, R₃, R₄ and R₅ are each H and the concentration of the phosphino-borane composition is 0.5M and 0.001M

In some embodiments, in the phosphorous boron-bonded compound M is Li, R₁ and R₂ are methoxy groups, R₃, R₄ and R₅ are each H and the concentration of the phosphino-borane composition is 0.5M and 0.001M

In some embodiments, in the phosphorous boron-bonded compound M is Li, R₁ and R₂ are both F, R₃, R₄ and R₅ are each H and the concentration of the phosphino-borane composition is 0.5M and 0.001M.

In some embodiments, in the phosphorous boron-bonded compound M is Li, R₁ and R₂ are both C₆F₅ groups, R₃, R₄ and R₅ are each H and the concentration of the phosphino-borane composition is 0.5M and 0.001M

In some embodiments, in the phosphorous boron-bonded compound M is Li, R₁ and R₂ are both —N(CH₃)—C═(NCH₃)—N(CH₃)₂ groups, R₃, R₄ and R₅ are each H and the concentration of the phosphino-borane composition is 0.5M and 0.001M

In some embodiments, in the phosphorous boron-bonded compound M is Li, R₁ and R₂ are both dimethylamino groups, R₃, R₄ and R₅ are each H and the concentration of the phosphino-borane composition is 0.5M and 0.001M.

In some embodiments, in the phosphorous boron-bonded compound M is Na, R₁ and R₂ are methyl groups, R₃, R₄ and R₅ are each H and the concentration of the phosphino-borane composition is 0.5M and 0.001M.

In some embodiments, in the phosphorous boron-bonded compound M is K, R₁ and R₂ are methyl groups, R₃, R₄ and R₅ are each H and the concentration of the phosphino-borane composition is 0.5M and 0.001M.

In some embodiments, in the phosphorous boron-bonded compound M is Na, R₁ and R₂ are phenyl groups, R₃, R₄ and R₅ are each H and the concentration of the phosphino-borane composition is 0.5M and 0.001M.

In some embodiments, in the phosphorous boron-bonded compound M is K, R₁ and R₂ are phenyl groups, R₃, R₄ and R₅ are each H and the concentration of the phosphino-borane composition is 0.5M and 0.001M.

In some embodiments, in the phosphorous boron-bonded compound M is Li, R₁ is a phenyl group, R₂ and R₃ are connected by a 3 carbon chain, R₄ and R₅ are each H and the concentration of the phosphino-borane composition is 0.5M and 0.001M

In some embodiments, in the phosphorous boron-bonded compound M is Li, R₁ is a phenyl group, R₂ and R₃ are connected by a 4 carbon chain, R₄ and R₅ are each H and the concentration of the phosphino-borane composition is 0.5M and 0.001M.

In some embodiments, in the phosphorous boron-bonded compound M is Li, R₁, R₂, R₃ and R₄ are methyl groups and R₅ is H and the concentration of the phosphino-borane composition is 0.5M and 0.001M.

In some embodiments, in the phosphorous boron-bonded composition, the vehicle is a solvent and the mixing is performed by dissolving in the solvent the at least one phosphorous boron-bonded compound to obtain a solution wherein the at least one phosphorous boron-bonded compound is dispersed within the solvent.

In some embodiments, in the phosphorous boron-bonded composition, the vehicle is an oil and the mixing is performed by dispersing in the oil the at least one phosphorous boron-bonded compound to obtain a dispersion wherein the at least one phosphorous boron-bonded compound is dispersed within the oil.

In some embodiments, in the phosphorous boron-bonded composition, wherein the vehicle is a solid matrix is performed by contacting the at least one phosphorous boron-bonded compound with the solid matrix to obtain a modified matrix wherein the at least one phosphorous boron-bonded compound is incorporated on the solid matrix.

A phosphorous boron-bonded composition of the disclosure comprising one or more phosphorous boron-bonded compounds of the disclosure can be manufactured by a method comprising mixing the at least one phosphorous boron-bonded compound with the suitable vehicle to obtain a phosphorous boron-bonded composition in which the at least one phosphorous boron-bonded compound is contained in the vehicle.

In some embodiments, the present disclosure relates to a phosphino-borane composition comprising a phosphino-borane-carbon dioxide complex within a suitable vehicle, wherein the phosphinoborane-carbon dioxide complex is a complex of Formula V.

In some of the embodiments in which the phosphino-borane composition comprises a phosphino-borane-carbon dioxide complex of Formula V, the composition is obtained with a method and/or system to capture CO₂ according to the present disclosure.

In some of the embodiments in which the phosphino-borane composition comprises a phosphino-borane-carbon dioxide complex of Formula V, the vehicle is a solvent and the mixing is performed by dissolving in the solvent the phosphinoborane-carbon dioxide complex to obtain a solution wherein the phosphino-borane-carbon dioxide complex is dispersed within the solvent.

In some of the embodiments in which the phosphino-borane composition comprises a phosphino-borane-carbon dioxide complex of Formula V, the vehicle is an oil and the mixing is performed by dispersing in the oil the phosphinoborane-carbon dioxide complex to obtain a dispersion wherein the at phosphino-borane-carbon dioxide complex is dispersed within the oil.

In some of the embodiments in which the phosphino-borane composition comprises a phosphino-borane-carbon dioxide complex of Formula V, the vehicle is a solid matrix is performed by contacting the phosphinoborane-carbon dioxide complex with the solid matrix to obtain a modified matrix wherein the phosphino-borane-carbon dioxide complex is incorporated on the solid matrix.

In some embodiments devices are also described that are configure to perform anyone of the method described herein.

In some embodiments devices are described that are configured for capturing carbon dioxide from a fluid. The device or system comprise:
a) a capture inlet configured to receive the fluid;
b) a capture reaction chamber in fluid communication with the capture inlet, the capture reaction chamber containing a carbon-dioxide-absorbing material;
c) a capture outlet in fluid communication with the capture reaction chamber, configured to discharge the phosphine-borane-carbon-dioxide complex from the capture reaction chamber.

The term “chamber”, as used herein indicates any enclosure that allows for a reaction between chemicals, such as a tank, cell, or well.

In the device, the carbon-dioxide-absorbing material is further configured to present at least one phosphorous boron-bond compound for contacting with carbon dioxide gas (CO2) from the fluid passing through the capture reaction chamber to produce a phosphine-borane-carbon-dioxide complex of Formula V.

In some embodiments the device configured for capturing carbon dioxide from a fluid, further comprises:
d) a phosphine-borane compound input in fluid communication with the capture reaction chamber, configured to input the phosphine-borane compound into the capture reaction chamber.

In some embodiments the device configured for capturing carbon dioxide from a fluid, further comprises:
e) an exhaust outlet in fluid communication with the capture reaction chamber configured to expel the fluid after having at least a portion of the carbon dioxide removed.

In some embodiments, in the device configured for capturing carbon dioxide from a fluid, the fluid is a gas and the carbon-dioxide-absorbing material is a liquid solution of the phosphine-borane compound in a base.

In some embodiments, in the device configured for capturing carbon dioxide from a fluid: the fluid is a gas or a liquid and carbon-dioxide-absorbing material is a solid.

In some embodiments, in the device configured for capturing carbon dioxide from a fluid: the solid comprises at least one flat surface comprising the phosphine-borane compound.

In some embodiments, in the device configured for capturing carbon dioxide from a fluid: the solid comprises beads comprising the phosphine-borane compound.

In some embodiments, in the device configured for capturing carbon dioxide from a fluid, the solid comprises a mesh or honeycomb structure comprising the phosphine-borane compound.

In some embodiments, in the device configured for capturing carbon dioxide from a fluid: the solid further comprises a polymer material, a silica or alumina material, a zeolite, or a metal-organic framework.

In some embodiments the device configured for capturing carbon dioxide from a fluid, further comprises one or more fans and/or pumps and/or valves configured to control flows through inlets, outlets, and/or the capture reaction chamber.

In some embodiments, a device is described for capturing carbon dioxide from a fluid, which comprises
a) a conversion inlet configured to receive a phosphine-borane-carbon-dioxide complex of Formula V;
b) a conversion reaction chamber in fluid communication with the release inlet, the conversion reaction chamber being configured to convert the phosphine-borane-carbon-dioxide complex of Formula V into the CO₂ reaction product of Formula VI in presence of a reducing agent and/or an alkylating agent; and
c) a conversion outlet in fluid communication with the release reaction chamber, configured to discharge the CO₂ reduction product of Formula VI from the conversion reaction chamber.

In some embodiments, the device for capturing carbon dioxide from a fluid, further comprises
d) a carbon dioxide reduction product outlet in fluid communication with the conversion reaction chamber, configured to output the carbon-dioxide reaction product from the conversion reaction chamber.

In some embodiments, in the device for capturing carbon dioxide from a fluid further comprising the conversion reaction chamber:
the reducing agent configured in the conversion reaction chamber to convert the phosphinoborane-carbon dioxide complex of Formula V into the carbon dioxide reduction product of Formula VI.

In some embodiments, the device for capturing carbon dioxide from a fluid further comprises:
electrodes configured in the conversion reaction chamber to convert the phosphinoborane-carbon dioxide complex into the carbon-dioxide product.

In some embodiments, in the device for capturing carbon dioxide from a fluid the electrodes comprise platinum, copper or alloys thereof.

In some embodiments, in the device for capturing carbon dioxide from a fluid the product is formic acid, methanol, acetic acid and/or ethanol.

In some embodiments, the device for capturing carbon dioxide from a fluid, further comprises one or more fans and/or pumps and/or valves configured to control flows through inlets, outlets, and/or the conversion reaction chamber.

In some embodiments the device for converting carbon dioxide in a fluid into a carbon dioxide reaction product, comprises:
a) a capture inlet configured to receive the fluid;
b) a capture reaction chamber in fluid communication with the capture inlet, the capture reaction chamber containing a carbon-dioxide-absorbing material comprising at least one phosphorous boron-bond compound selected from a phosphine-borane of Formula I, a cyclic phosphine-borane of Formula III and a phosphine-borane salt of Formula IV;
c) a capture outlet in fluid communication with the capture reaction chamber, configured to discharge the phosphine-borane-carbon-dioxide complex of Formula V from the capture reaction chamber into a release inlet configured to receive the phosphine-borane-carbon-dioxide complex of Formula V;
d) a conversion reaction chamber in fluid communication with the release inlet, the release reaction chamber being configured to convert the phosphine-borane-carbon-dioxide complex of Formula V into carbon dioxide reaction product of Formula VI; and
e) a conversion outlet in fluid communication with the release reaction chamber, configured to discharge the carbon dioxide reaction product of Formula VI from the release reaction chamber.

In the device, the carbon-dioxide-absorbing material is configured to present at least one phosphorous boron-bond compound for contacting with carbon dioxide gas (CO2) from the fluid passing through the capture reaction chamber to produce a phosphine-borane-carbon-dioxide complex of Formula V.

In some embodiments, a device for scrubbing carbon dioxide out of a fluid, comprise:
a) an inlet for the fluid into the device;
b) a filter comprising at least one phosphorous boron-bond compound selected from a phosphine-borane of Formula I, a cyclic phosphine-borane of Formula III and a phosphine-borane salt of Formula IV, the filter configured to present at least one phosphorous boron-bond compound for contacting with carbon dioxide gas from the fluid passing through the capture reaction chamber to produce a phosphine-borane-carbon-dioxide complex of Formula V; and
c) an outlet expelling the fluid from the device after passing through the filter, the fluid have at least a portion of the phosphine-borane-carbon-dioxide complex of Formula V removed from the filter.

In some embodiments, a device for capturing carbon dioxide from a fluid, the device comprise:
a) a capture inlet configured to receive the fluid;
b) a capture reaction chamber in fluid communication with the capture inlet, the capture reaction chamber containing a carbon-dioxide-absorbing material; and
c) a capture outlet in fluid communication with the capture reaction chamber, configured to discharge the phosphinoborane-carbon dioxide complex from the capture reaction chamber,
   in which the carbon dioxide-absorbing material comprises at least one phosphorous-boron bond compound selected from a phosphine-borane of Formula I, a cyclic phosphine-borane of Formula III and a phosphine-borane salt of Formula IV, and
   in which the carbon dioxide-absorbing material is configured to present at least one phosphorous-boron bond compound for contacting with carbon dioxide gas (CO2) from the fluid passing through the capture reaction chamber to produce a phosphinoborane-carbon dioxide complex of Formula V.

In some embodiments, the device can further comprise:
d) a phosphine-borane compound input in fluid communication with the capture reaction chamber, configured to input the phosphine-borane compound into the capture reaction chamber.

In some embodiments, the device can further comprise:
e) an exhaust outlet in fluid communication with the capture reaction chamber configured to expel the fluid after having at least a portion of the carbon dioxide removed.

In some embodiments, of the device, the fluid is a gas and the carbon-dioxide-absorbing material is a liquid solution of the phosphine-borane compound in a base.

In some embodiments, of the device, the fluid is a gas or a liquid and carbon-dioxide-absorbing material is a solid.

In some embodiments, of the device, the solid comprises at least one flat surface comprising the phosphine-borane compound.

In some embodiments, of the device, the solid comprises beads comprising the phosphine-borane compound.

In some embodiments, of the device, the solid comprises a mesh or honeycomb structure comprising the phosphine-borane compound.

In some embodiments, of the device, the solid further comprises a polymer material, a silica or alumina material, a zeolite, or a metal-organic framework.

In some embodiments, the device further comprise one or more fans and/or pumps and/or valves configured to control flows through inlets, outlets, and/or the capture reaction chamber.

In some embodiments, a device for converting a phosphinoborane-carbon dioxide complex, into a carbon dioxide reaction product, comprise:
a) a conversion inlet configured to receive a phosphine-borane-carbon-dioxide complex of Formula V;
b) a conversion reaction chamber in fluid communication with the release inlet, the release reaction chamber being configured to convert the phosphine-borane-carbon-dioxide complex of Formula V into the CO₂ reaction product of Formula VI in presence of a reducing agent and/or an alkylating agent; and
c) a conversion outlet in fluid communication with the release reaction chamber, configured to discharge the CO₂ reduction product of Formula VI from the release reaction chamber.

In some embodiments, the device further comprises
d) a carbon dioxide reduction product outlet in fluid communication with the release reaction chamber, configured to output the carbon-dioxide reaction product from the release reaction chamber.

In some embodiments, the device further comprises in the release reaction chamber:
the reducing agent configured in the release reaction chamber to convert the phosphinoborane-carbon dioxide complex of Formula V into the carbon dioxide reduction product of Formula VI.

In some embodiments, the device further comprises
electrodes configured in the release reaction chamber to convert the phosphinoborane-carbon dioxide complex into the phosphine-borane compound and the product.

In some embodiments of the device, the electrodes comprise platinum, copper or alloys thereof.

In some embodiments, the device further comprises one or more fans and/or pumps and/or valves configured to control flows through inlets, outlets, and/or the release reaction chamber.

In some embodiments a device for scrubbing carbon dioxide out of a fluid, comprises:
a) an inlet for the fluid into the device;
b) a filter comprising at least one phosphorous boron-bond compound selected from a phosphine-borane of Formula I, a cyclic phosphine-borane of Formula III and a phosphine-borane salt of Formula IV,
   the filter configured to present at least one phosphorous boron-bond compound for contacting with carbon dioxide gas from the fluid passing through the capture reaction chamber to produce a phosphine-borane-carbon-dioxide complex of Formula V; and
c) an outlet expelling the fluid from the device after passing through the filter, the fluid have at least a portion of the phosphine-borane-carbon-dioxide complex of Formula V removed from the filter.

In some embodiments the devices further comprises: a wash inlet configured to flow a reducing agent through the filter, converting the phosphine-borane-carbon-dioxide complex of Formula V into carbon dioxide reaction product of Formula VI and a product outlet configured to output the carbon dioxide reaction product of Formula VI.

In some embodiments of the device, the filter comprises at least one flat surface comprising the phosphine-borane compound.

In some embodiments of the device, wherein the filter comprises beads comprising the phosphine-borane compound.

In some embodiments of the device, the filter comprises a mesh or honeycomb structure comprising the phosphine-borane compound.

In some embodiments of the device, the filter further comprises a one or more of polymer material, a silica or alumina material, a zeolite, and a metal-organic framework.

As described herein, phosphorous boron-bonded compounds, vehicles, reducing agents, alkylating agents equipment and/or devices herein described can be provided as a part of systems to perform any methods, including any the methods described herein.

In some embodiments, a system for converting carbon dioxide in a fluid into a carbon dioxide reaction product, comprises:
a) a capture inlet configured to receive the fluid;
b) a capture reaction chamber in fluid communication with the capture inlet, the capture reaction chamber containing a carbon-dioxide-absorbing material comprising at least one phosphorous-boron bond compound selected from a phosphine-borane of Formula I, a cyclic phosphine-borane of Formula III and a phosphine-borane salt of Formula IV;
c) a capture outlet in fluid communication with the capture reaction chamber, configured to discharge the phosphinoborane-carbon dioxide complex of Formula V from the capture reaction chamber into a release inlet configured to receive the phosphinoborane-carbon dioxide complex of Formula V;
d) a conversion reaction chamber in fluid communication with the release inlet, the release reaction chamber being configured to convert the phosphinoborane-carbon dioxide complex of Formula V into carbon dioxide reaction product of Formula VI; and
e) a conversion outlet in fluid communication with the release reaction chamber, configured to discharge the carbon dioxide reduction product of Formula VI from the release reaction chamber.
   wherein the carbon-dioxide-absorbing material is configured to present at least one phosphorous-boron bond compound for contacting with carbon dioxide gas (CO₂) from the fluid passing through the capture reaction chamber to produce a phosphine-borane-carbon-dioxide complex of Formula V.

In some embodiments the system further comprises:
f) a phosphorous boron-bond compound inlet in fluid communication with the capture reaction chamber, configured to input the phosphine-borane compound into the capture reaction chamber.

In some embodiments the system further comprises:
g) an exhaust outlet in fluid communication with the capture reaction chamber configured to expel the fluid after having at least a portion of the carbon dioxide removed.

In some embodiments of the system: the fluid is a gas and the carbon dioxide-absorbing material is a liquid solution of the phosphine-borane compound in a base.

In some embodiments of the system: the fluid is a gas or a liquid and carbon dioxide-absorbing material is a solid.

In some embodiments of the system: the solid comprises at least one flat surface comprising the phosphine-borane compound.

In some embodiments of the system: the solid comprises beads comprising the phosphine-borane compound.

In some embodiments of the system: the solid comprises a mesh or honeycomb structure comprising the phosphine-borane compound.

In some embodiments of the system: the solid further comprises a one or more of polymer material, a silica or alumina material, a zeolite, and a metal-organic framework.

In some embodiments the system further comprises: one or more fans and/or pumps and/or valves configured to control flows through inlets and/or outlets and/or the capture reaction chamber and/or the release reaction chamber.

In some embodiments the system further comprises: in the release reaction chamber:
the reducing agent configured in the release reaction chamber to convert the phosphine-borane-carbon-dioxide complex of Formula V into the carbon dioxide reaction product of Formula VI.

In some embodiments the system further comprises:
electrodes configured in the release reaction chamber to convert the phosphinoborane-carbon dioxide complex into the phosphine-borane compound and the product.

In some embodiments of the system: the electrodes comprise platinum, copper or alloys thereof.

The system of claim 18, further comprising a phosphine-borane compound storage tank in fluid communication with the phosphine-borane inlet configured to provide phosphine-borane compound to the capture reaction chamber.

In some embodiments the system further comprises: a product reaction chamber in fluid communication with the release outlet and configured to process the carbon dioxide reaction product of Formula VI into a further product.

In some embodiments of the system: the further product comprises plastic usable in production of plastic items or plastic components.

Further details concerning the phosphorous-boron bonded compounds which are configured for capturing CO₂ and related products, compositions, methods and systems for capturing storing and/or utilizing cycling carbon dioxide including generally manufacturing and packaging of the compounds compositions, devices and systems can be identified by the person skilled in the art upon reading of the present disclosure.

### EXAMPLES

The phosphorous-boron bonded compounds which are configured for capturing CO₂ and related products, compositions, methods and systems for capturing storing and/or utilizing cycling carbon dioxide, are further illustrated in the following examples, which are provided by way of illustration and are not intended to be limiting.

A skilled person will be able to identify additional phosphorous-boron bonded compounds which are configured for capturing CO₂ and related products, compositions, methods and systems for capturing storing and/or utilizing cycling carbon dioxide herein described in view of the content of the present disclosure. The following specific examples are given to illustrate the practice of the invention, but are not to be considered as limiting the invention in any way.

In particular, exemplary phosphorous-boron bonded compounds which are configured for capturing CO₂ and related products, compositions, methods and systems for capturing storing and/or utilizing cycling carbon dioxide are described in connection with specific experimental tests and procedures. A skilled person will be able to understand and identify the modifications required to adapt the results illustrated in the exemplary embodiments of this sections to additional embodiments of phosphorous-boron bonded compounds which are configured for capturing CO₂ and related products, compositions, methods and systems for capturing storing and/or utilizing cycling carbon dioxide within the present disclosure.

The following materials and methods were used for all compounds and their precursors exemplified herein.

### Computational Methods

All Density Functional Theory calculations were performed using the Jaguar v10.9 software from Schrodinger Inc. All calculations utilized the M06-2X meta-GGA functional with the D3 empirical correction for London Dispersion forces.^{21, 22}

All atoms were described by the 6-311G**++ Pople basis set,²³ augmented with polarization and diffuse functions.

All calculations included a continuum solvent treatment described by the Polarizable Continuum Model (CPCM).²⁴

Unless otherwise noted, we used solvent parameters matching tetrahydrofuran (dielectric constant=7.6, probe radius=2.52).

Vibrational frequency calculations were performed to confirm that all geometries are true minima (no negative eigenmodes of the Hessian) and to compute thermochemical properties such as enthalpy (H), entropy (S), and free energies (G) at 298.15 K. Because librational modes are hindered in solvent media, we corrected our free energies by reducing translational and rotational entropy modes by 50%.

Computationally, pKa is calculated in water solvent, following the equation pKₐ=D/(2.3RT), where D is the free energy change associated with dissociation of BH⁺ to B⁻ and H⁺ in water solvent. The value of H⁺ is determined to be −259.5 kcal/mol for the solvation free energy change of a proton.

### Example 1 Low pKa Phosphorus Boron Bonded Compounds for Strong Capture of Carbon Dioxide

The growing world population with dramatically increased energy needs has led to dramatic increases in atmosphere CO₂ concentrations that increase the risk of irreversible negative effects on the climate.^{1,2} One potential solution towards reducing atmospheric CO₂ levels is to capture the CO₂ and then convert it to a value-added product such as methanol, ethanol, or to a polymer feedstock such as ethylene. Amines and simple phosphide complexes are well established for binding CO₂ and serve as the basis for state-of-the-art CO₂ extraction technologies.^{3, 4}

In contrast phosphines generally do not form stable CO₂ adducts due primarily to their decreased basicity.⁵ A few notable exceptions are extremely electron-rich phosphines studied by Dielmann et al,⁶ as well as phosphide metal salts,⁷ all of which are exceptionally strong bases. Frustrated Lewis Pairs, first synthesized by Douglas et. al.,⁸ have also been analyzed for CO₂ capture⁹, with these compounds using a Frustrated Lone Pair induced by steric hindrance to drive CO₂ binding. These compounds do not include a P—B bond and instead use steric hindrance to drive binding.

While previous analyses have examined various Frustrated Lewis Pairs for CO₂ activation, in phosphorous-boron bonded compound of the disclosure direct formation of the P—B bond allows for the borane moiety to donate charge to the phosphorus. This allows for these compounds to have extensively different properties than those in the previous FLPs studied for CO₂ binding and sequestration. In addition, standard deprotonated phosphides and their salts have also been used. These compounds have exceedingly high binding energies toward CO₂ capture, although formation of their active analogs via deprotonation requires a strong base.

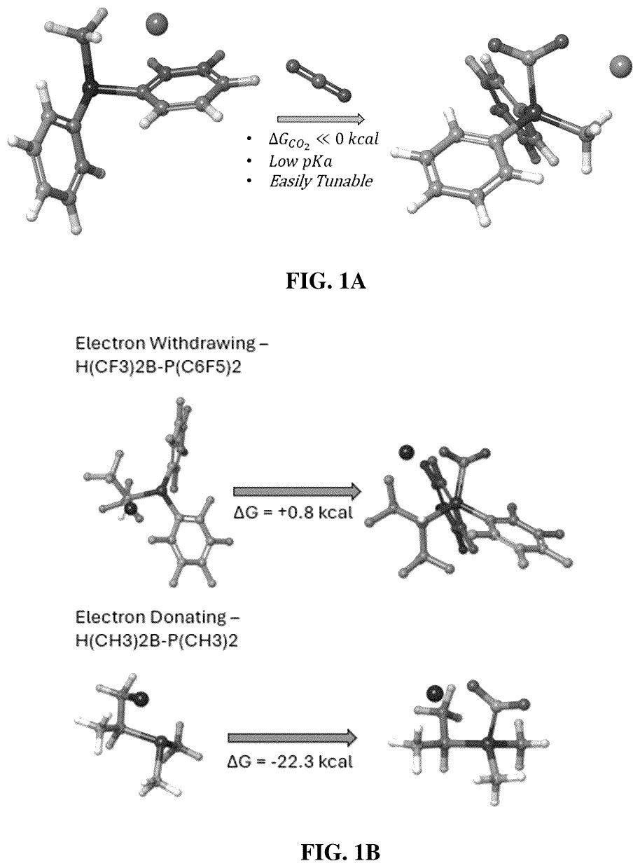

An exemplary schematic illustration of the phosphorous-boron bonded compounds of the present disclosure is reported in FIGS. 1A to 1C showing the effects of electron donating and withdrawing substituents on the boron and phosphorus moieties.

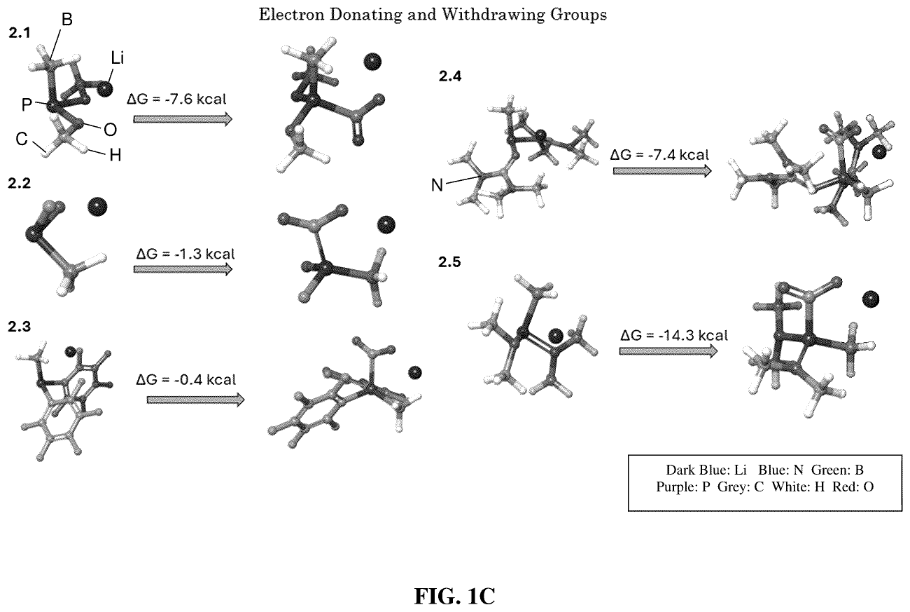

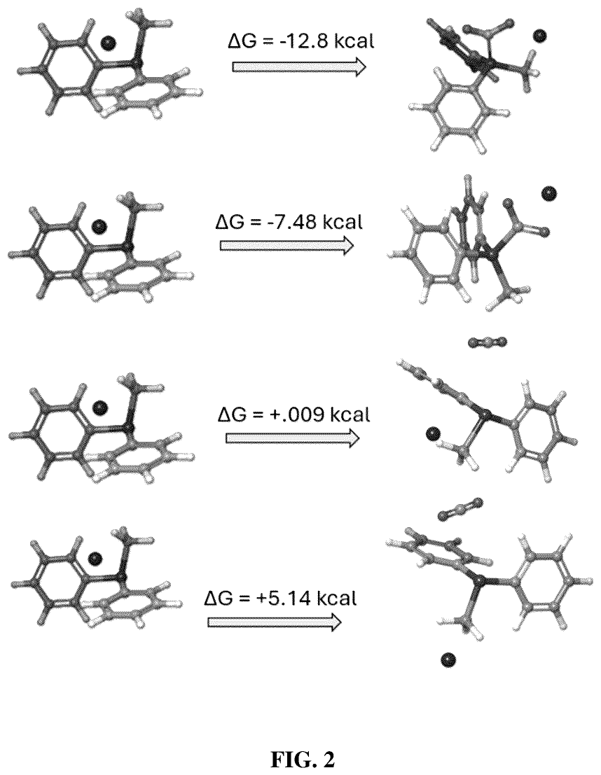

Additionally exemplary schematic illustrations of the phosphorous-boron bonded compounds of the present disclosure is reported in FIGS. 2 to 3 showing the effects of the counterion on the stability and binding energetics of exemplary phosphorous boron bonded compounds of the disclosure.

In particular in the schematic illustration of FIG. 2, the tight ion pairing with Li⁺ enhances the compound's stability by reducing the anion's reactivity and susceptibility to side reactions. This strong electrostatic interaction helps maintain the structural integrity of the compound over time. The distance has between the X⁺ and the binding pocket has a drastic effect on ΔG of CO₂ binding. As the distance is perturbed from the ideal 1.95 Å between X⁺ and BH₃⁻, and 1.81 Å between X⁺ and CO₂⁻, ΔG increases from −12.8 kcal/mol to −6.9 kcal/mol as the distance between the X⁺, (which, in this case, Li⁺) is adjusted by 0.35 Å

In the schematic illustration of FIG. 3, it is shown that Li⁺ significantly improves the energetics of CO₂ binding. This interaction: Reduces the energy of the CO₂-bound state, leading to a more favorable binding free energy (e.g., ΔG=−24.0 kcal/mol for ethyl-substituted compounds). And Bends the O—C—O angle from 1800 to approximately 130°, enhancing orbital overlap and strengthening the P—CO₂ bond.

The strong CO₂-binding affinity enabled by Li⁺ makes Li⁺ preferred for applications like direct air capture or permanent CO₂ sequestration, where maximizing capture strength is essential. The presence of a vehicle such as a solvent can affect this properties as will be understood by a skilled person.

For example phosphido-borane complexes, previously studied by Izod et. al. contain a P—B bond stabilized by a Li⁺ counterion position located at the borane moiety. The Li⁺ counterion is further stabilized by electron donation from THF, found in the structural characterization of the complex itself.¹⁰ Furthermore, analysis of the basicity of these complexes, completed by Dornhaus et. al. indicates that the anionic motif of the phosphido-borane salt is more Lewis basic towards the [BH₃] compared to its neutral analogs, making these compounds ripe for usage in CO₂ capture.¹¹

A class of Phosphido-Boranes (BoPh's) has been developed with formula X⁺[R₂PBH₃⁻] that bind CO₂ with exceptional strength (ΔG=−8.2 to −24.0 kcal/mol) at ambient conditions. Quantum Mechanics (QM) was to determine how the choice of electron-donating versus electron-withdrawing ligand impacts the CO₂ binding strength, in the presence of a donating borane moiety.

The role of the cation in CO₂ binding was examined, finding that the ion position relative to the bound CO₂ dramatically alters binding strength. It was found that the BoPh with two ethyl ligands Li[Et₂PBH₃] leads to a ΔG=−24.0 kcal/mol upon CO₂ binding while Li[Ph₂PBH₃] leads to ΔG=−12.8 kcal/mol.

Exemplary BoPh were synthesized with two phenyl ligands Li[Ph₂PBH₃] to validate the QM predicted stability and predicted pKa.

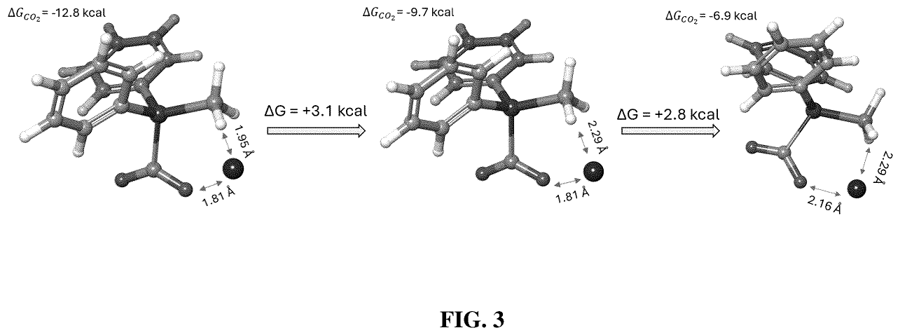

Exemplary reaction scheme for the use of the phosphorous-boron bonded compounds of the disclosure is reported in FIG. 4.

### Example 2; Identification of BoPh's Substituents and BoPh's Configuration for CO₂Capture

Herein, a combined computational-experimental study is reported that examines several phosphido-boranes (BoPh) for the efficient capture of CO₂. Quantum Mechanics (QM) was used to predict CO₂ binding for a number of BoPh's followed by NMR experimental validation in d₈-THF solution. The deprotonated secondary phosphido-boranes complexes exhibit CO₂ binding free energies of −8.2 to −24.0 kcal/mol at 300 K despite having only moderate Brønsted basicity, comparable to that of the tert-butoxide anion.

Formation of the Lewis acid-base complex between BH₃ and secondary phosphines (R₂P—H) leads to a substantial increase in the P—H Brønsted acidity of the H—PR₂—BH₃⁻ adduct compared to the free phosphine. This observation has been exploited widely in the synthesis of tertiary phosphines, since PR₂—BH₃⁻ anions serve as excellent P nucleophiles, bear a built-in BH₃ protecting group for the P atom, and offer significant experimental advantages compared to the parent R₂P⁻ phosphide counterpart.¹² These phosphide-borane salts have excellent CO₂ binding in comparison to other P-based systems as a result of their acidity.

Instead of a strong base needed for deprotonation and subsequent activation, only mild conditions are needed. While these phosphido-borane complexes have some ditopic character, with dual reactivity at the both the P and H centers, the use of these phosphide-borane complexes as hydride sources has not been reported. As shown by Consiglio et. al.,¹² the introduction of various carbonyl derivatives led to the formation of multiple products due to the inclusion at both the P site and H site in the compounds under investigation. This ditopic behavior indicates the complexity of the reaction mechanisms associated by reduction induced by various phosphide-borane compounds.¹³ What has so far remained unnoticed is that the PR₂—BH₃⁻ anions possess remarkably strong CO₂ binding affinity for P-based compounds with Brønsted basicity between that of bulky trialkylphosphines (e.g., PCy₃) and the weakest of the P superbases, such as Verkade's base, neither of which form stable CO₂ adducts. Furthermore, the CO₂ binding motif in the form of a single P—C bond in the resulting carboxylate ion rather than a cyclic lactone is likely to benefit subsequent activation to form stable CO₂ based compounds.

Here it Density Functional Theory (DFT) studies are reported and corresponding determinations followed examining several subclasses of phosphido-boranes (BoPh) candidates for use in CO₂ capture. We found that the ΔG of CO₂ binding ranges from −8.2 to −24.0 kcal/mol. Of the molecules studied, the BoPh with two phenyl ligands Li[Ph₂PBH₃] led to a predicted ΔG=−12.8 kcal/mol, which have now been validated experimentally.

The influence of electron-withdrawing and electron-donating groups on CO₂ capture and their interplay with the anionic borane motif was first evaluated. The counter-cation effect on CO₂ capture was also investigated, revealing how the position of the cation relative to the BoPh dramatically alters CO₂ binding strength. Described herein are several BoPh's with varying functionalities that can affect CO₂ binding. BoPh's 1, 2, and 3 contain electron-donating groups, and BoPh 4 contains an electron-withdrawing group. Phosphines 5 and 6 do not contain the borane moiety, serving as references for comparison with BoPh's 1 through 4. While it is known that simple phosphide salts form CO₂ complexes, included example phosphines were included as reference to explore the effect of the borane unit on CO₂ binding energies. This is shown in the case of phosphine 5.

In particular,
1 [(CH₃)₂PBH₃] contains two —CH₃ (Me) groups which should function as weak electron-donating groups
2 [(CH₃CH₂)₂PBH₃] contains two —CH₂CH₃ (Et) groups, as an extension to 1
3 [(Butyl)₂PBH₃] contains the BoPh anion (P—BH₃⁻) accompanied by two n-butyl units bound to the central P. Butyl is used here because it was previously found that both Me and Et groups had extremely borane, such that the third ligand position becomes occupied by the counter-cation
6 [Ph₃P] also lacks the borane and should therefore serve nicely for elucidating the effect of the borane moiety on CO₂ binding affinity
The reactant conformer was selected by varying the position of the counterion across multiple local pockets in the reactant complex. From there, the ion position that led to the lowest-energy conformation was selected. CO₂ binding free energies for the lowest-energy reactant and product conformers with Li⁺ counterions are shown in FIG. 2. It was observed that the closer the counterion was to the P—BH₃ exergonic CO₂ binding strength (−23.1 and −24.0 kcal/mol). Moreover, it is possible to directly compare the butyl groups to the Me and Et groups for CO₂ binding, in the context of altering the chain lengths of unsubstituted alkyl groups to an electron-rich substrate such as the BoPh.
4 [(Ph)₂PBH₃] has two —C₆H₅(Ph) units, which should operate as electron-withdrawing groups bonded to the central, electron-rich P
5 [(Ph)₂P] also has two Ph units but lacks the unit, the more able it was to stabilize charge and thus the more effectively it was able to substantially increase reactant stability.

### Example 3; Identification of BoPh's Counterions and BoPh's Configuration for CO₂Capture

The overall ΔG of binding CO₂ is based on the stability of the product complex. The counterion plays a significant role in stabilizing the CO₂⁻ moiety upon binding, balancing charge distribution throughout the entire system. Altering the placement of the counterion in different pockets throughout the system dramatically changes binding energies. Here, the closer the counterion is to the bounded CO₂ and the electron rich moieties of the system, including the electron donating —BH₃ unit, the more able it is to stabilize effective charge. Shown below, a summary of the effect of the counterion in terms of overall binding energies is shown by changing the pocket where each counterion is coordinated to the system. As shown in the image, the pocket where the counterion is coordinated to the system dramatically affects binding energy.

Absolute distance also affects binding energy. In the first case, where the Li⁺ counterion is positioned between the terminal oxygen and the —BH₃ group, increasing the distance by merely 0.35 angstroms increases binding energy by +5.9 kcal. This has been shown in FIG. 3.

In the illustration of FIG. 3 the effect of distance on ΔG are shown. Increasing the counterion distance from the O—BH₃ pocket by 0.35 Å increases binding energy by +5.9 kcal. ΔG_{CO2} increases for the biphenyl case from −12.8 kcal/mol to −6.9 kcal/mol.

For the case of Li⁺ counterions, the most stable product BoPh conformer has the Li⁺ seated between an oxygen of the bound CO₂ and two hydrides of the anionic —BH₃—. Increases in the thermodynamic driving force as a result of this can be attributed to charge stabilization in the product structure, as well as charge transfer from P to the CO₂ unit upon binding¹⁴. In these structures, binding CO₂ leads the borane moiety to donate a charge of +0.09 to the P—C single bond (P is now formally +1). A general finding was that bulkier ligands increase steric crowding which in turn limits the availability of the nucleophilic P for binding CO₂.

As shown for compound 4, the coupled effect of having both weak electron-withdrawing groups and steric crowding around the nucleophilic site of the phosphorus lone pair decreases the magnitude of ΔG, although the anionic borane offsets this effect due to charge transfer into the newly formed P—C bond.

In compounds 1, 2, and 3, alkyl groups act as weak electron donors to the nucleophilic P binding site, leading to ΔG from −24 to −19 kcal/mol.

In BoPh 4, the reactant P charge is much higher than those BoPh 1-3. The larger cationic character of the phosphorus in the reactant complex limits the ability for nucleophilic attack on the electrophilic carbon of the CO₂, decreasing the ΔG to −13 kcal/mol. Compared to other conformers in which the counterion was placed in less stable sites, Li⁺ coordination to the oxygen of the CO₂ unit and the hydrides of the borane (FIG. 2) yielded O—C—O bond angles close to sp² hybridization (120°). Strong deviation from the linear bond angle of 1800 for CO₂ also indicates increased thermodynamic driving force for CO₂ binding, with more negative ΔG values arising from species able to accommodate CO₂ near 120°. As the Li⁺ moves closer to the oxygens, the O—C—O bond angle decreases to near the sp² hybridization angle of 120°.

Li⁺, along with its role in adjusting the CO₂ bond angle, acts as a soft Lewis Acid in the THF solvent, making ΔG more negative. The naked biphenyl phosphide-borane has an incredibly downhill binding energy of almost −12 kcal/mol for CO₂ binding. Other analogs have even stronger binding energies, some in the −20 to −30 kcal/mol range. This result can be used as a baseline for further alternations to the ligands bound to phosphorus to increase overall ΔG for conversion to a value-added product. For the creation of a value-added product, ΔG for CO₂ binding must be increased in order to ensure that the bound CO₂ can be released upon formation of the product.

This can be done in a variety of different ways, including
decreasing the nucleophilicity of the phosphorus by increasing the electron withdrawing nature of the ligands, e.g. by introducing —F substituents on the aryl withdrawing group or otherwise,
decreasing the electron donating ability of the —BH₃ group by replacing the hydrides with other substituents,
altering reaction conditions to make conditions more acidic, or
changing the counterion involved in stabilizing the bound CO₂ product, such as replacing Li⁺ with K⁺.

This would ultimately balance adsorption and collection ΔGs for capture and subsequent conversion.

### Example 4; Effect of Solvents on BoPh's Configuration for CO₂ Capture

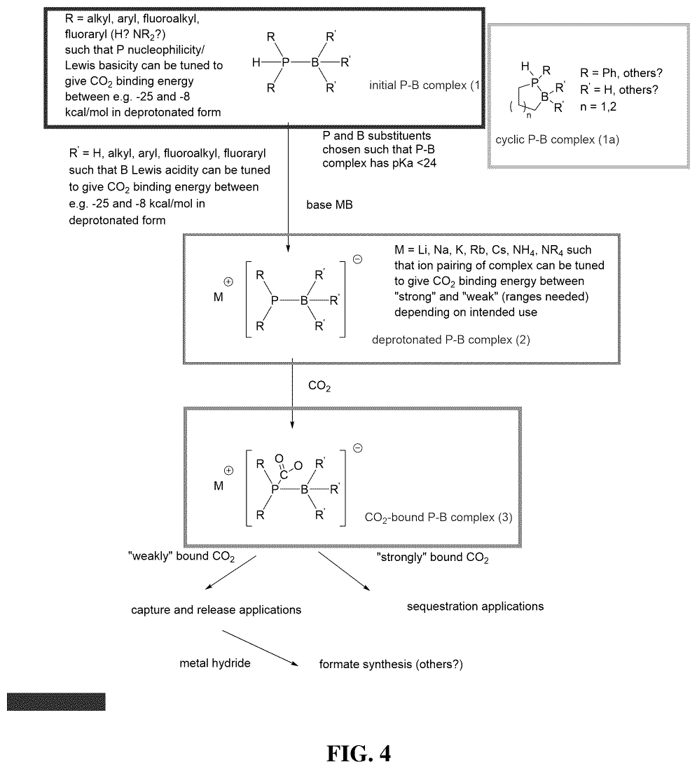

Explicit solvent inclusion in the reactant conformers indicates that, in addition to stabilizing effects from both the phosphorus (P) and boron (B) atoms due to the counterion being placed between them, the electron-donating tetrahydrofuran (THF) molecules also contribute to stabilization (see FIG. 5).

The oxygens of each THF group are oriented towards the counterion, enhancing the overall stability of the system. This has been validated through x-ray crystallography of the reactant structures, and is in direct agreement with previous analyses of these phosphido-borane complexes in THF and other electron-donating solvent.^{10, 12}

However, while inclusion of these stabilization effects is necessary for understanding the system, they are minimal in their effect on CO₂ capture. The additional stability provided by the THF groups only increases ΔG of CO₂ binding by 3.0 kcal/mol.

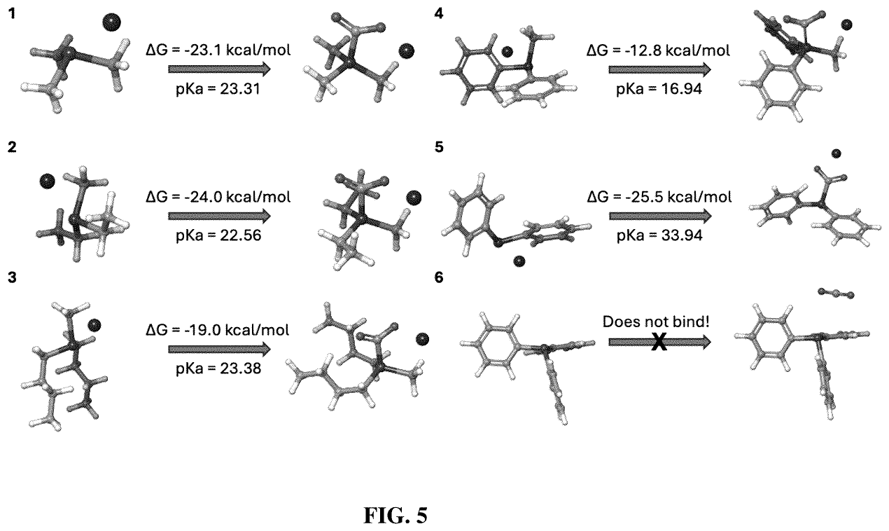

This has been elucidated in comparing CO₂ binding in [(Ph)₂PBH₃] using DFT. This is shown in FIG. 6:

Table 5: Properties of various BoPh's For the case of a Li⁺ counterion in THF. CO₂ binding free energy (kcal/mol), charges on the P atom and BH₃⁻ moiety in both reactant and product complexes, O—C—O bond angle after binding, P—B and P—C bond orders (BO) and bond lengths (Å).

TABLE 5
Properties of various BoPh's

| Phosphido- | ΔG for | ΔQ on | ΔQ on | CO₂ | Change in P—B | P—C |
|---|---|---|---|---|---|---|
| borane | CO₂ binding | P | BH₃⁻ | Charge | Bond Order | Bond Order |
| [(CH₃)₂PBH₃] | −23.1 | +0.73 | +0.088 | −0.992 | −0.067 | +0.82 |
| [(CH₃CH₂)₂PBH₃] | −24.0 | +0.74 | +0.086 | −0.970 | −0.074 | +0.81 |
| [(Butyl)₂PBH₃] | −19.0 | +0.72 | +0.082 | −0.987 | −0.079 | +0.84 |
| [(Ph)₂PBH₃] | −12.8 | +0.57 | +0.082 | −0.942 | −0.061 | +0.84 |

In regard to the effect of the counterion on CO₂ stability, while the charge of the Li⁺ counterion remains unchanged in compounds 1 through 5, Li⁺ polarizes the oxygens of the CO₂ unit in the product, slightly disrupting otherwise perfect resonance among both 0 atoms of the CO₂-bound complex.

### Example 5: pKa of BoPh and Binding Affinity for CO₂ Capture

The pKa of the phosphido-borane conjugate acid is correlated to the binding affinity of CO₂ to the central phosphorus, with higher pKa values indicating a greater Lewis basicity and hence greater reactivity to the Lewis acidity of the CO₂. This can be seen in differences between phosphines 1, 2, 3, with pKa=23 and 4 with pKa=17. The relative acidity of the H—P⁺R₂BH₃⁻ complex compared to the naked H—PPh₂ requires only a mild base to form the active lone pair site on the phosphorus. Here, the borane moiety increases the acidity of the hydrogen on the H—P⁺R₂BH₃⁻ zwitterionic complex acting as a source of electron charge upon CO₂ binding, effectively making ΔG less negative and increasing synthetic ease.

Along with the correlation of the O—C—O bond angle with ΔG values, the zwitterion character changes. While no conclusions can be made about the correlation between zwitterionic character and the thermodynamic driving force of CO₂ binding, zwitterion formation can provide insight as to the mechanism of binding and the origin of such high ΔG values. The BH₃⁻ group present in each BoPh complex acts as a source of electron density upon CO₂ binding, with the Lewis basicity of each P atom increasing upon binding due to the donation of charge from the BH₃⁻ moiety. Replacing the BH₃⁻ moiety with the —R group ligand decreases the ΔG values for all tested species. Replacing BH₃⁻ with —Ph, as shown for 6, increased ΔG of CO₂ binding significantly, to the point where CO₂ binding was no longer favorable.

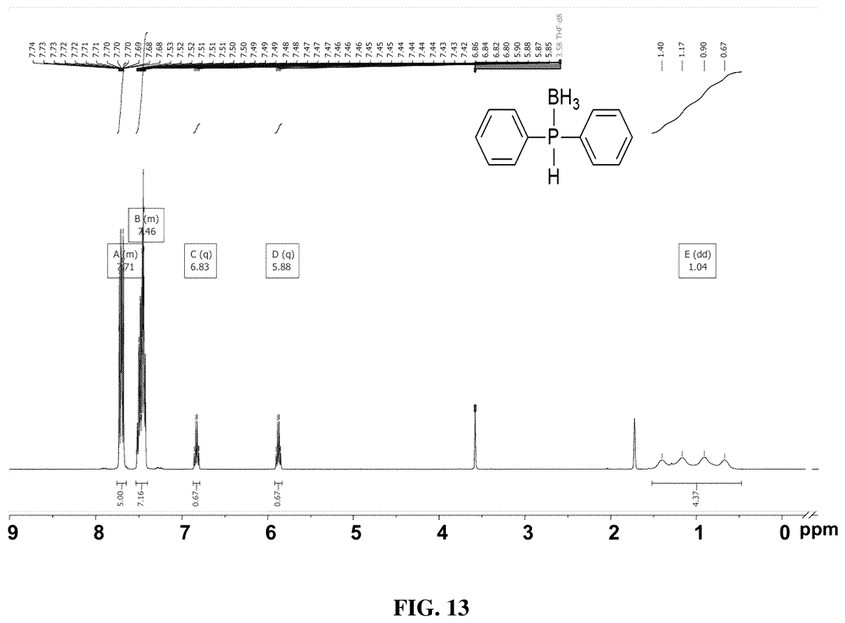

For comparison, CO₂ binding strengths was also computed in the presence of a Na⁺ counterion. Reactant and product structures along with their respective CO₂ binding energies are depicted in FIG. 13 of the Supplementary Information (SI)

Na⁺ is a softer ion than Li⁺ in terms of its cationic character. As a result, charge stabilization in the Na⁺ product complex is not as pronounced as for Li⁺ case. Lack of stabilization in the product conformer reduces the stability of the product complex, making ΔG less negative, as illustrated in FIG. 13. Decreased charge stabilization in the product complex also increases the transition state barrier (ΔGt) for CO₂ binding. In both the Na- and Li-containing transition states, the counterion is positioned between the approaching CO₂ and the BH₃⁻ moiety of the phosphido-boranes. The counterion forms an ion-pair with the negatively charged BH₃⁻ subunit, simultaneously accepting charge from the partially charged oxygens of the now-binding CO₂. In addition, the decreased polarization in the transition state reduces the overall energy of the system, reducing the large differences in polarization in the activated complex. Decreased charge polarization subsequently reduces the transition state energy, and thus the overall activation barrier.

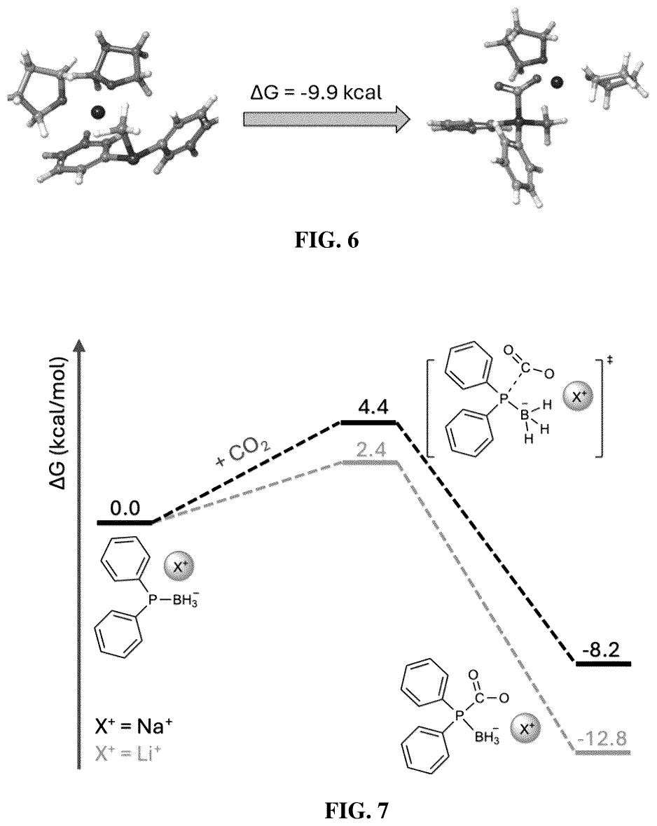

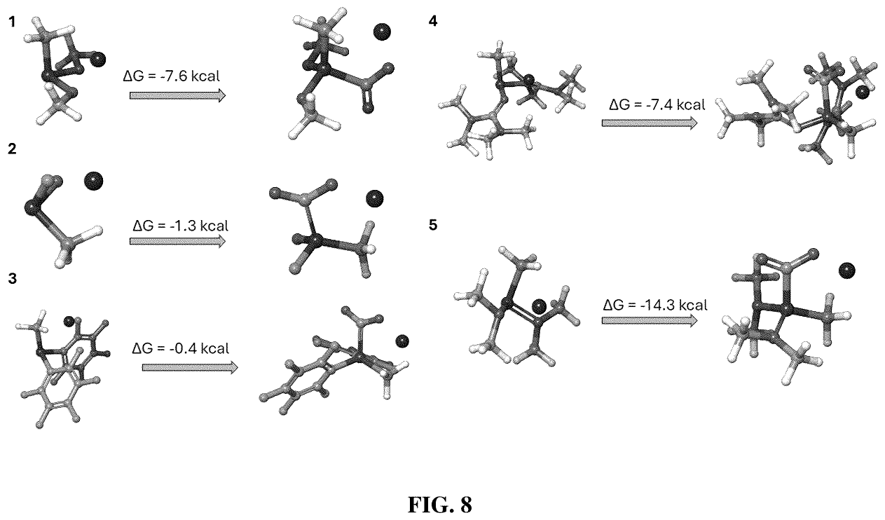

The transition state and binding free energies for [(Ph)₂PBH₃] with Li⁺ and Na⁺ are depicted in FIG. 7; here the position of the counterion is of critical importance. In FIG. 7, it was observed that Li⁺ coordinated to a CO₂ oxygen and the hydrides of the BH₃⁻ reduces ΔG‡ by ˜2 kcal/mol relative to Na⁺. The overall CO₂ binding ΔG for the Li⁺- and Na⁺-containing phosphido-boranes are +2.43 and +4.43 kcal/mol, respectively. The ΔG‡ of CO₂ binding for the parent Li—PPh₂ and Na—PPh₂ which lack the borane are +0.7 and +1.5 kcal/mol, respectively.

### Example 6; Competing BH₃ Transfer Reactions and BoPh's Configuration for CO₂ Capture

While direct pathways of CO₂ binding are almost instantaneous, competing BH₃ transfer reactions must also be considered. BH₃⁻ transfer from THF-BH₃ to PPh₂BH₃ for the formation of Na⁺PPh₂(BH₃)₂ is unfavorable in comparison to CO₂ binding, with a ΔGt of +9.6 kcal/mol. Formation of Na⁺PPh₂BH₃⁻ via BH₃ transfer from BH₃THF to Na⁺PPh₂⁻ in the absence of CO₂ occurs with a barrier of +10.9 kcal/mol; this reaction occurs instantaneously in experiment (see below).

In addition to Na⁺ and Li⁺, PPh₄⁺ was also used as a counterion in our experiments. Because of the bulky and rigid nature of PPh₄⁺, CO₂ was unable to bind to the phosphido-boranes complex, with PPh₄⁺ forming a salt complex with the reactant phosphine instead.

This, alongside FIG. 3 and Tables 5 and FIG. 13, highlight the significance of the counterion in binding. To separately control the transition barrier and adsorption energy, alterations to the counterion and the system can be done separately to alter one or the other.

As shown by the schematic illustration of FIG. 3 The most stable of the counterion is in-between the CO₂ and P—BH₃ groups. Moving the counterion to other groups such as near the —BH₃ or the —CO₂⁻ groups drive ΔG significantly, to −7.5 and +5.1 kcal respectively

In particular, as shown in FIG. 3, substituting Li⁺ for Na⁺ increases ΔG of adsorption while only slightly increasing the transition state barrier. This decreases the stability of the product complex without necessarily affecting the transition state barrier significantly.

Similarly, if one were to use a sterically hindered counterion, such as a quaternary carbon or nitrogen, one could raise the transition state barrier while keeping the binding energy unchanged. The phosphorus would be unavailable to attack the Lewis Acidic CO₂, and thus the activation energy would increase without necessarily altering the binding energy if an equivalently stabilizing counterion were to be used. Adjusting either the ligand or the counterion in regards to steric control and counterion alterations could selectively adjust either the transition state barrier or the adsorption energy, respectively. To shed further insight into the role of the counterion, electron-donating, and electron-withdrawing groups in CO₂ binding, additional phosphine complexes were examined with other donating and withdrawing ligands, as shown in FIG. 5 and Table 6.
(CH₃O)₂PBH₃ (FIG. 4-1) contains two —CH₂OH units, which should operate as weak electron withdrawing groups. The position of the oxygen atoms in the methoxy groups stabilize the counterion, decreasing CO₂ binding.
F₂PBH₃ (FIG. 4-2) has two —F groups. The F explores the effects of an inductive electron withdrawing group. The —F R-group withdraws electron density away from the nucleophilic phosphorus atom. This makes ΔG significantly less negative.
(F₅C₆)₂PBH₃ (FIG. 4-3) has two —C₆F₅ groups, acting as electron withdrawing aryl groups, again removing charge from the P and leading to much less favorable ΔG.
(TMG)₂PBH₃ (FIG. 4-4) contains the phosphido-borane anion (P—BH₃) accompanied by two monodentate tetramethyl guanidium (TMG) ligands bound to the central P. TMG is used here because it was previously found that P(TMG)₃¹⁵ had a very exergonic CO₂ binding strength (−13.5 kcal/mol), however here ΔG=−7.4 kcal/mol.
[(H₃C)₂N]₂PBH₃ (FIG. 4-5) contains two —N(CH₃)₂ ligands which act as electron donating groups to the nucleophilic phosphorus, leading to ΔG=−14.3 kcal/mol

Table 6: Binding energy of CO₂ binding, and respective charges on the P atom and BH₃⁻ moiety or the Li⁺ counterion in THF in both reactant and product complexes P—B and P—C bond orders (BO). The ΔG of CO₂ binding and ΔQs are also shown here as well.

TABLE 6
Binding energy of CO₂ binding, and charges

| Phosphido- | ΔG for | ΔQ on | ΔQ on | CO₂ | Change in P—B | P—C Bond |
|---|---|---|---|---|---|---|
| borane | CO₂ binding | P | BH₃⁻ | Charge | Bond Order | Order |
| (CH₃O)₂PBH₃ | −7.6 | +0.74 | +0.06 | −1.03 | −0.09 | +0.78 |
| F₂PBH₃ | −1.3 | +0.65 | +0.02 | −0.96 | −0.11 | +0.82 |
| (F₅C₆)₂PBH₃ | −0.4 | +0.49 | +0.11 | −0.85 | −0.07 | +0.84 |
| (TMG)₂PBH₃ | −7.4 | +0.78 | +0.01 | −1.09 | −0.02 | +0.73 |
| [(H₃C)₂N]₂PBH₃ | −14.3 | +0.77 | +0.03 | −1.03 | −0.05 | +0.78 |

### Example 7: Effects of Substituents and Counterions on BoPh's Configuration for CO₂ Capture

As found in the alkyl BoPh's, ΔG is correlated to ΔQ on P. Cases with the more negative ΔG have larger ΔQ values, regardless of whether the ligand type present is electron-donating or electron-withdrawing. However, electron-donating groups on the phosphine did yield lower ΔG values than electron-withdrawing groups, highlighting the significance of ligand choice is BoPh synthesis. Higher negative charge on the CO₂ moiety is correlated with a lower ΔG. Interestingly, smaller values of ΔQ on BH₃⁻ are correlated with lower ΔG values. (F₅C₆)₂PBH₃, with two electron-withdrawing C₆F₅ groups, exhibits a large change in ΔQ upon CO₂ binding. Contrasted with [(H₃C)₂N]₂PBH₃ containing two electron-donating —N(CH₃)₂, ΔQ on BH₃⁻ in (F₅C₆)₂PBH₃ was significantly higher, by +0.08.

This trend is not observed in the alkyl and aryl BoPh's, indicating that electron-donating and electron-withdrawing ligand groups drastically impact the ability of BH3- to donate charge and stabilize the +1 charge on phosphorus. ΔQ is the change in the charge on the phosphorus and —BH₃ moieties, upon formation of the (+1) tetravalent phosphorus upon binding of the CO₂. A higher ΔQ is indicative of the relative nucleophilicity of the phosphorus in context of a potent Lewis Acid such as CO₂. In the case of CO₂ binding, if ΔQ is larger, then the reactant is very nucleophilic and is able to bind CO₂ easier as compared to compounds where ΔQ is far smaller. For BH₃, upon binding CO₂, charge is donated from the BH₃ to the P—C bond. This increases ΔG and is what is responsible for the incredibly high ΔG values shown here.

Electron donating groups increase the nucleophilicity and electron density on the phosphorus, increasing its ability of nucleophilic attack on the electrophilic carbon center of CO₂. This then decreases ΔG. Electron-withdrawing groups decrease the electron density at phosphorus and hence its nucleophilicity. This then decreases the magnitude of ΔQ on P, resulting in smaller overall change in electron density between product and reactant structures. In addition to charge density, there is a correlation between bond order and ΔG. BoPh's with little to no difference in reactant and product P—B bond orders have more negative ΔG values, as opposed to BoPh's with larger differences in P—B bond orders. The resulting difference between the product and reactant conformers is also responsible for increased ΔG values. As a result of the increased stability of the Li⁺ counterion in the (TMG)₂PBH₃ complex from the nitrogens, the reactant complex is more stable than it otherwise would be without the nitrogens. This then decreases overall reactivity and increases ΔG.

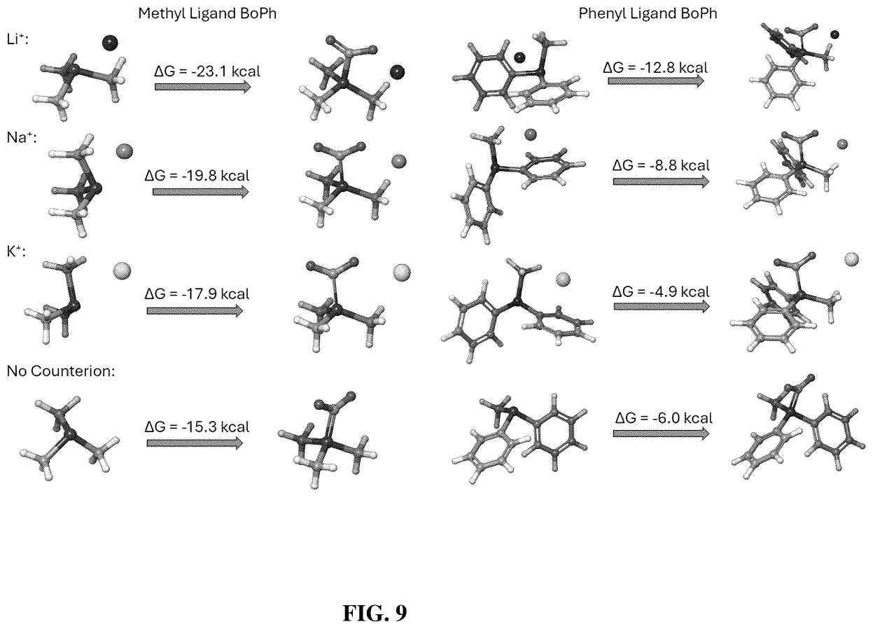

The counterion stabilizes effective charge buildup in both the product and transition state, resulting in more negative overall ΔG values. Furthermore, changes in the relative size of the counterion drastically affects ΔG, as shown for the Na⁺Na⁺, and K⁺ cases in FIG. 9.

The counterion stabilizes charge in the product and transition state, making ΔG more negative. Here, it was observed that due to the larger size of Na⁺, it is unable to stabilize charge as well as Li⁺. This is also seen for K⁺. In addition, for the no counterion cases with methyl and phenyl R-groups, there is no stabilization of charge in the entire anionic complex, with ΔG hence being the most positive. Between Li⁺ and the cases with no counterion, a large difference in ΔG was observed. Thus, between the methyl and phenyl cases, ΔG increases by an average of +7.3 kcal.

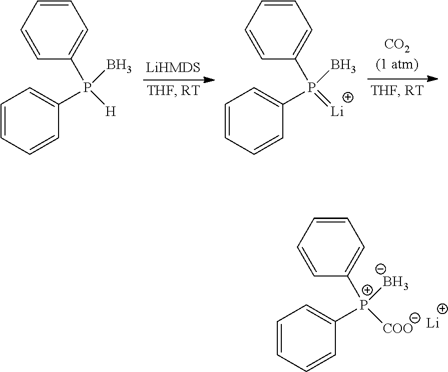

### Example 8: Synthesis of Diphenylphosphine Borane Complex (Ph₂PHBH₃)

Very few studies have been published on Phosphines-based system that bind CO₂^{16, 17, 18} These systems either exhibit very strong basicity or create electron density on the P atom, making it electron-rich. For instance, the works of Buβ et al., showcases electron-rich phosphines that bind CO₂, where their most stable adducts or best phosphines have predicted pKa value of ˜33.7 and free binding energy at room temperature of −10.3 kcal/mol¹⁶. In contrast our BoPh's have predicted binding free energies up to 23 kcal/mol with pKa as low as 17. To validate the computational studies, the anionic diphenylphosphido-borane, 4, was synthesized since it had the lowest pKa and a good binding energy as described in Example 6.

Diphenylphosphine Li[Ph₂PBH₃] borane complex was thus synthesized by by following the synthetic route described by Stankevic et. al¹⁹.

A solution of BH₃·SMe₂ (0.545 ml, 5.75 mmol, in 2 ml of hexanes) was added dropwise to a solution of diphenylphosphine (1 ml. 5.75 mmol, in 2 ml of hexanes) at −15° C. in a 20 ml valve. The mixture was swirl and shaken to allow homogenous mixture. In about 10-20 mins, crystal growth of the desired product could see in the valve. The mother liquor was transferred into a clean valve to collect all crystals. The crystals were washed several times with hexanes and evaporated to dryness. The crystals were then furthered characterized with NMR. Yield 71.04%.

Here 0.2218 g of the resulting phosphido-borane was dissolved in 3 ml of tetrahydrofuran (THF) in a 10 ml valve. Here 2 ml of THF was used to dissolve 0.1856 g of lithium bis(trimethylsilyl)amide (LiHMDS). The solution of LiHMDS was added drop wise to the solution of Li[Ph₂PBH₃] to minimize side reactions.

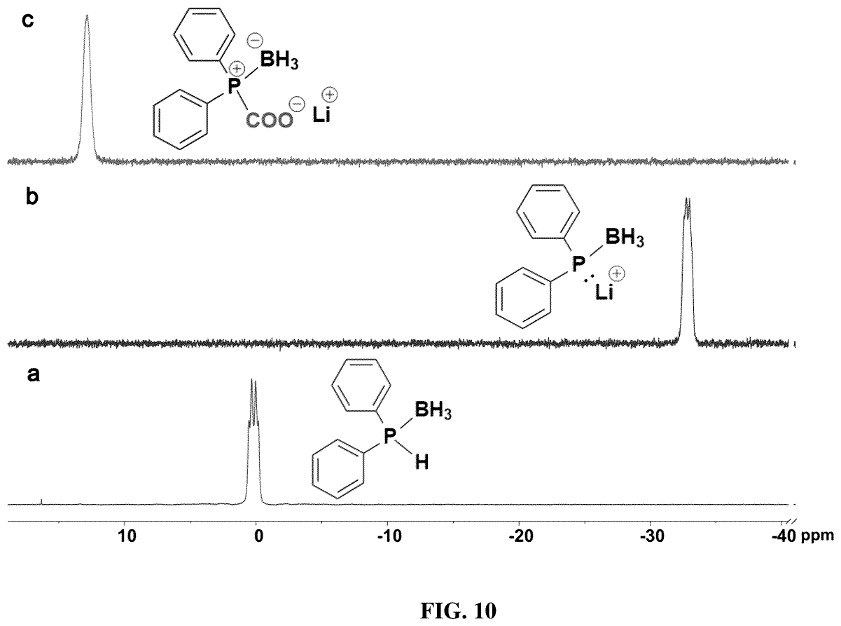

An aliquot of the resulting solution was taken to confirm the structure using NMR spectroscopy with the results in FIG. 10 panel b, confirming complete deprotonation Li[Ph₂PBH₃] to form Li[Ph₂PBH₃] salt.

The NMR spectra reported FIG. 10 shows
a) ³¹P NMR of Li[Ph₂PBH₃](red spectra), (162 MHz, THF-d₈) δ 1.16-−0.79 (m).
b) ³¹P NMR of LiPh₂PBH₃(blue spectra), (162 MHz, THF-d₈) δ −32.87 (d, J=60.7 Hz)
c) ³¹P NMR of LiPh₂PBH₃—CO₂(green spectra), (162 MHz, THF-d₈) δ 12.86

CO₂ gas was then bubbled through lithiated anionic phosphido-borane salt at 1 atm and room temperature for about 10 mins to saturate it with CO₂. NMR analysis on an aliquot of the sample revealed formation of P—CO₂ adducts as shown in FIG. 10 Panel c. For confirmation of the predicted pKa of the P Li[Ph₂PBH₃], lithium tertbutoxide was used to deprotonate the Li[Ph₂PBH₃] in butanol which resulted in the lithiated anionic phosphido-borane salt. This leads to a pKa of 19.2 in good agreement with the predicted pKa of 21.3 for lithium tert-butoxide, which is consistent with the lower pKa of 17 for Ph₂BoPh.

FIG. 10 shows the ³¹P NMR resonance spectra of Ph₂BoPh (FIG. 10 Panel
a), Li[Ph₂PBH₃] salt (FIG. 10Panel b) and the Li[Ph₂PBH₃]—CO₂ adduct (Figure. 10 Panel c).

The spectra in FIG. 10 Panel a and FIG. 10 Panel b show a quartet resonance signal due to ³¹P-¹¹B coupling. The upfield chemical shift from 0.19 ppm to −32.87 ppm indicates successful deprotonation of the phosphido-borane complex to form its lithiated salt. This upfield shift is expected as a result of the shielding effect of the lone pair on the phosphorous atom which is very much in accordance with the works by Jaska et al²⁰. The downfield chemical shift from −32.87 ppm to 12.86 ppm in FIG. 10 Panel c due to the CO₂ molecule bound to the phosphorus atom results from deshielding of the phosphorus nuclei by the Lewis acid, which in this case is the CO₂ gas molecule. The broad characteristic peak feature of the ³¹P NMR of the Li[Ph₂PBH₃]—CO₂ adduct is likely the result of an exchanging the CO₂ from one phosphorous atom to another.

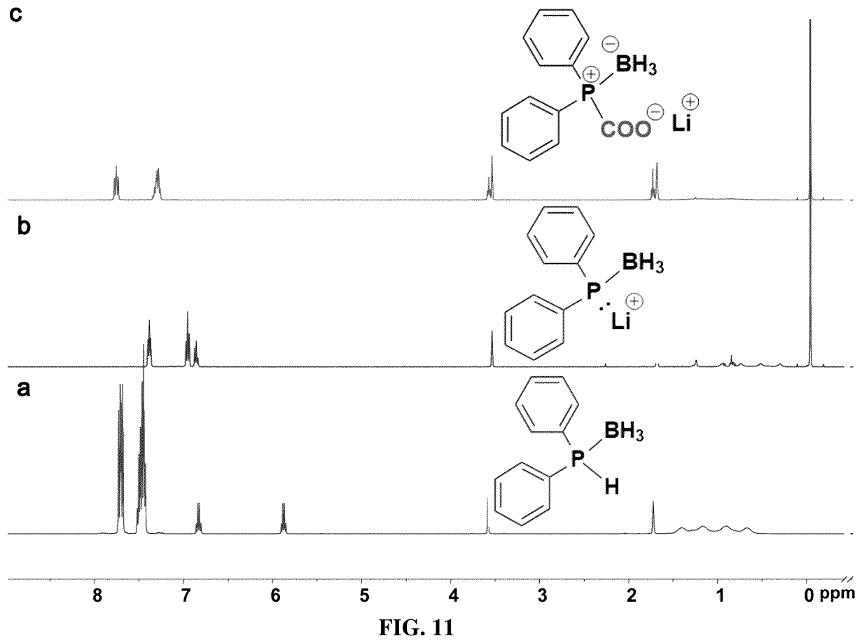

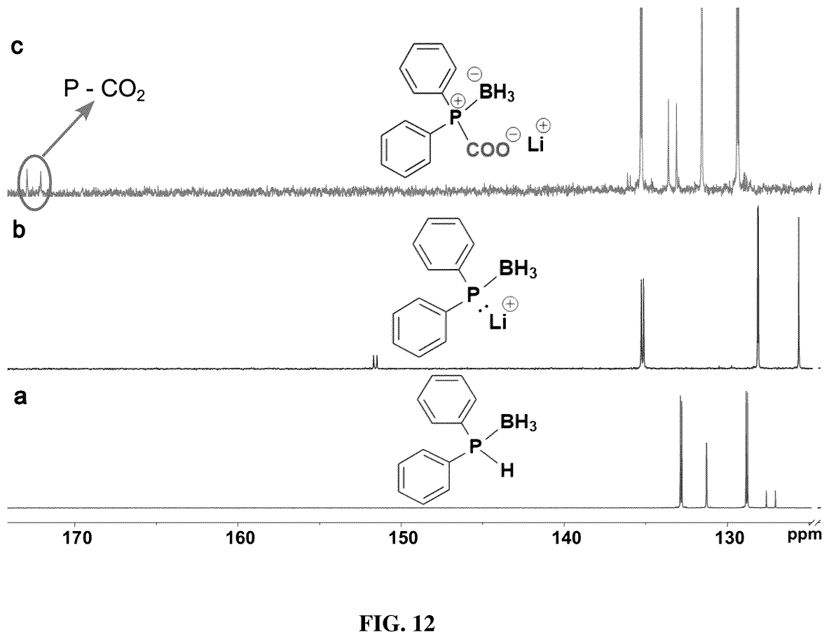

FIGS. 11 and 12 depicts the ¹H and ¹³C NMR of the Ph₂PHBH₃ (red spectra), LiPh₂PBH₃(blue) and LiPh₂PBH₃—CO₂ (green spectra) respectively.

The disappearance of the P—H bond at 6.83 and 5.88 ppm in FIG. 10 (blue spectra) shows the complete formation of LiPh₂PBH₃ which serves as the nucleophilic material that reacts with CO₂ to form the LiPh₂PBH₃—CO₂ (green spectra). As shown in FIG. 10 (green spectra), the P atom does not steal any H from the BH₃ unit but rather binds successfully with the CO₂ molecule to form the adduct. This is further confirmed by the ¹³C NMR in FIG. 12 (green spectra), where the appearance of the signals at 172.95 and 172.11 ppm shows the coupling of the P atom to the CO₂ molecule from the LiPh₂PBH₃—CO₂ complex.

### Example 9 Synthesis of Diphenylphosphine Borane Complex (Ph₂PHBH₃)

A solution of BH₃·SMe₂ (0.545 ml, 5.75 mmol, in 2 ml of hexanes) was added dropwise to a solution of diphenylphosphine (1 ml. 5.75 mmol, in 2 ml of hexanes) at −15° C. in a 20 ml valve. The mixture was swirl and shaken to allow homogenous mixture. In about 10-20 mins, crystal growth of the desired product could see in the valve. The mother liquor was transferred into a clean valve to collect all crystals. The crystals were washed several times with hexanes and evaporated to dryness. The crystals were then furthered characterized with NMR. Yield 71.04%.

The detected spectra are

¹H NMR (400 MHz, THF-d₈) δ 7.75-7.65 (m, 5H), 7.53-7.40 (m, 7H), 6.83 (q, J=7.1 Hz, 1H), 5.88 (q, J=7.1 Hz, 1H), 1.04 (dd, J=199.4, 94.5 Hz, 4H). (see FIG. 13)

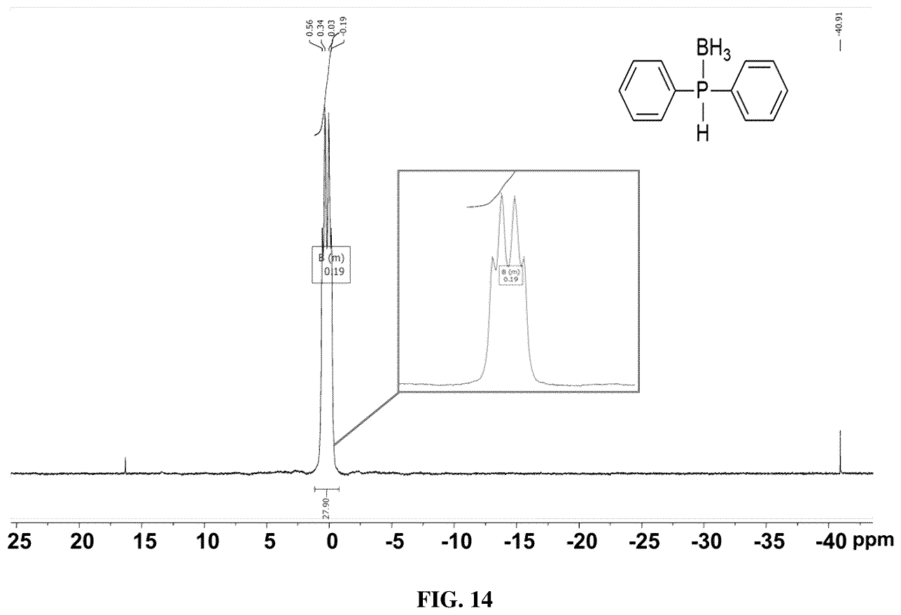

³¹P NMR (162 MHz, THF-d₈) δ 1.16-−0.79 (m). (see FIG. 14)

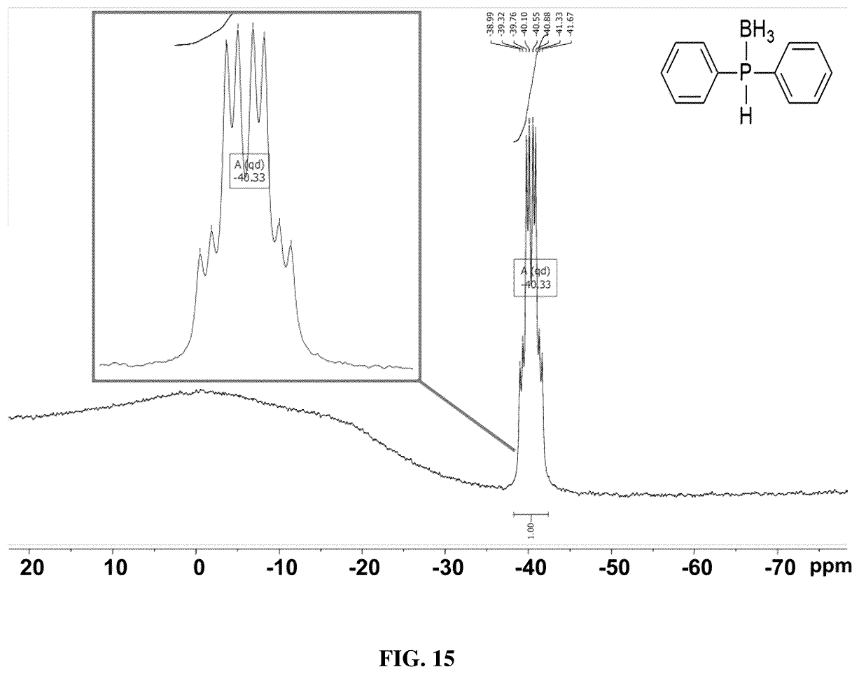

¹¹B NMR (128 MHz, THF-d₈) δ −40.33 (qd, J=101.1, 100.0, 43.3 Hz). (see FIG. 15)

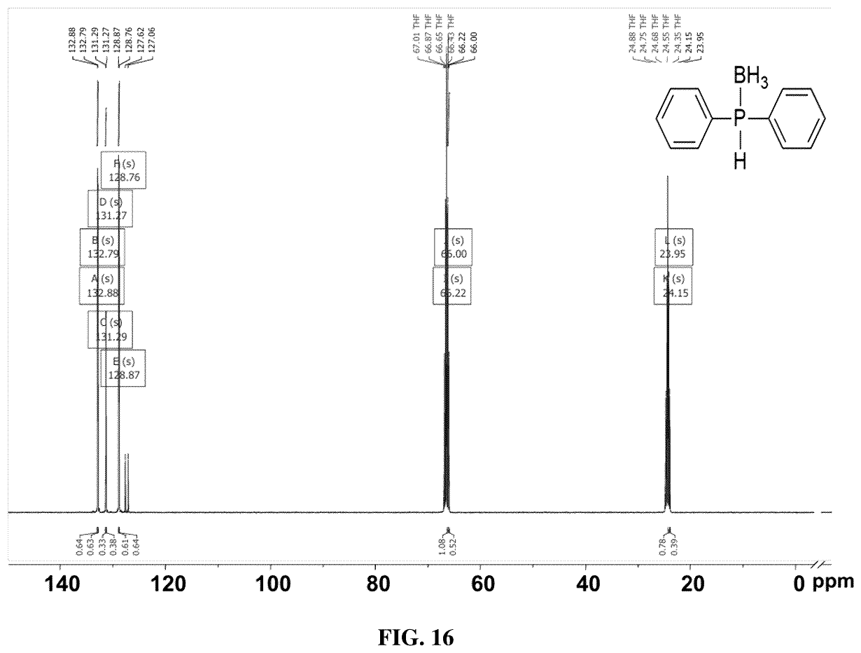

¹³C NMR (101 MHz, THF-d₈) δ 132.88, 132.79, 131.29, 131.27, 128.87, 128.76, 66.22, 66.00, 24.15, 23.95 (see FIG. 16)

### Example 10: Synthesis of Anionic Diphenylphosphine Borane Complex M(Ph₂PBH₃)

To obtain instant precipitate of the product, a solution of Ph₂PHBH₃ (0.586 mmol, in 4 ml of toluene) was added dropwise to solution of MHMDs where M represent either Li or Na or K (0.586 mmol, in 4 ml of toluene) which resulted in an instant precipitation of the product. The product was then filtered. The precipitate on the filtered frit was washed with hexanes several times and allowed to dried in the glovebox for 2 days. Yield 89.6%.

NMR analysis for Na(Ph₂PBH₃) salt provided the following results

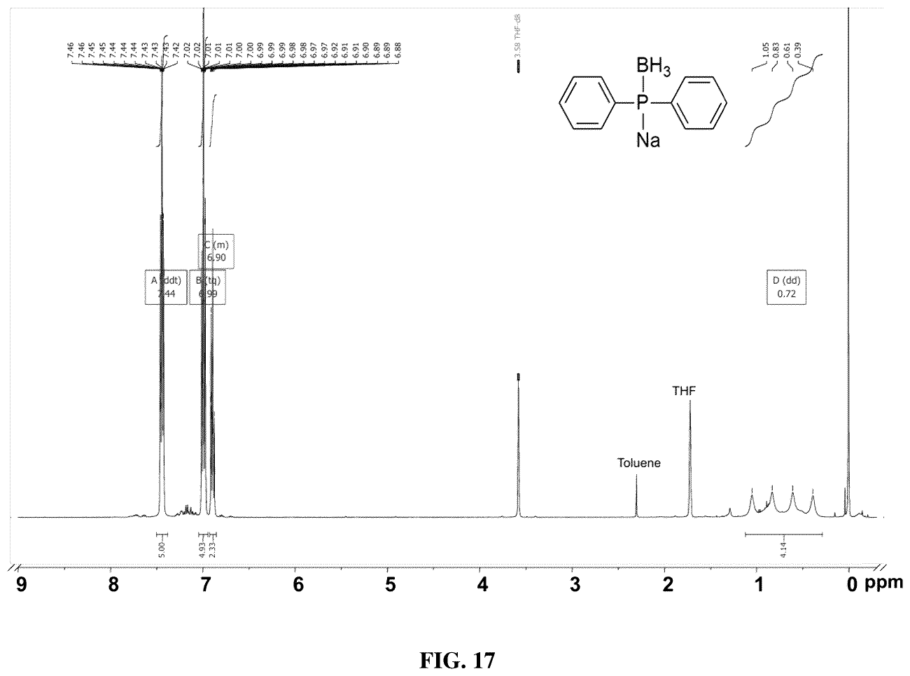

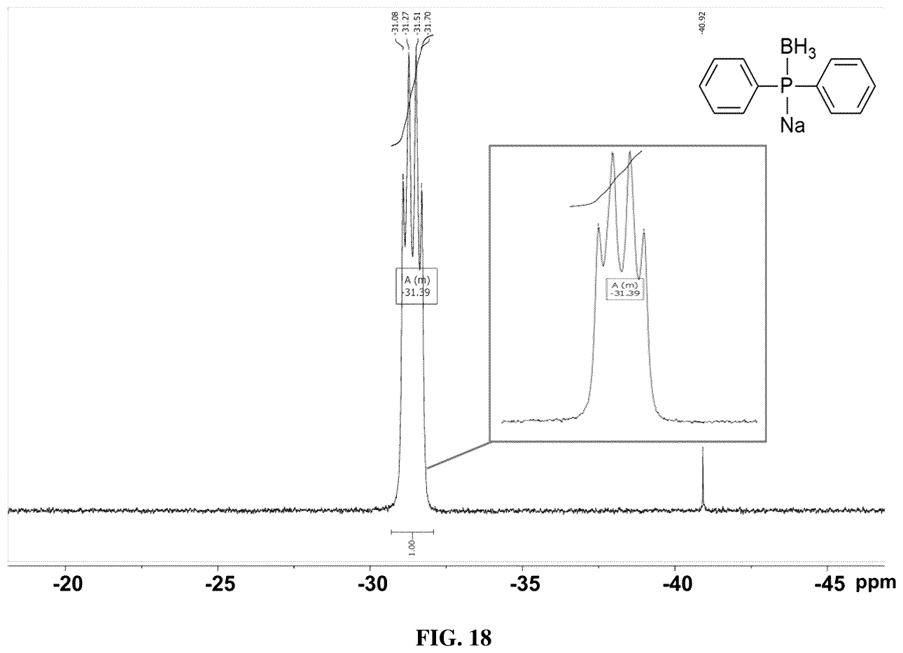

¹H NMR (400 MHz, THF-d₈) δ 7.44 (ddt, J=7.2, 6.0, 1.4 Hz, 5H), 6.99 (tq, J=6.7, 0.9 Hz, 5H), 6.93-6.85 (m, 2H), 0.72 (dd, J=176.9, 87.4 Hz, 4H). (see FIG. 17)

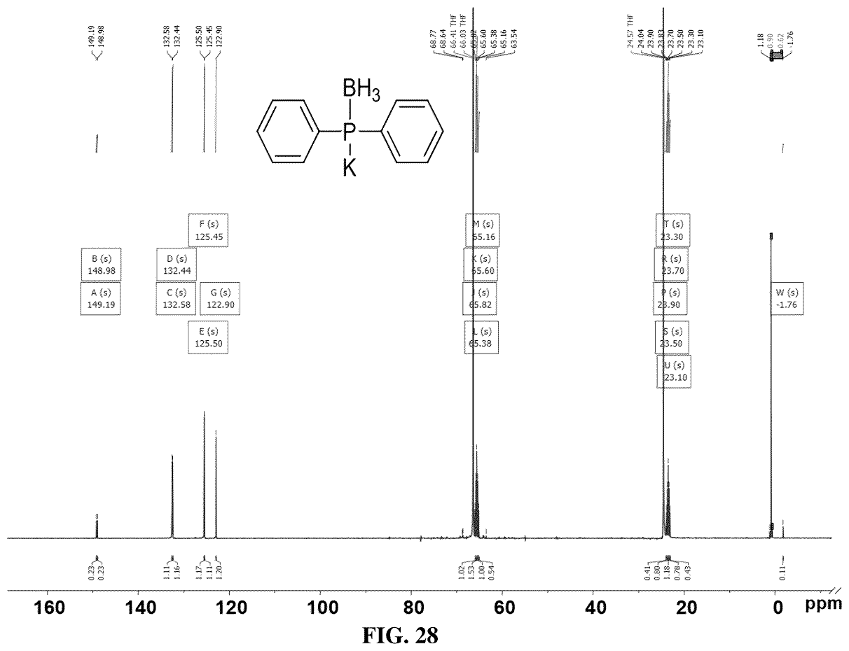

³¹P NMR (162 MHz, THF-d₈) δ −30.69-−32.08 (m). (see FIG. 28)

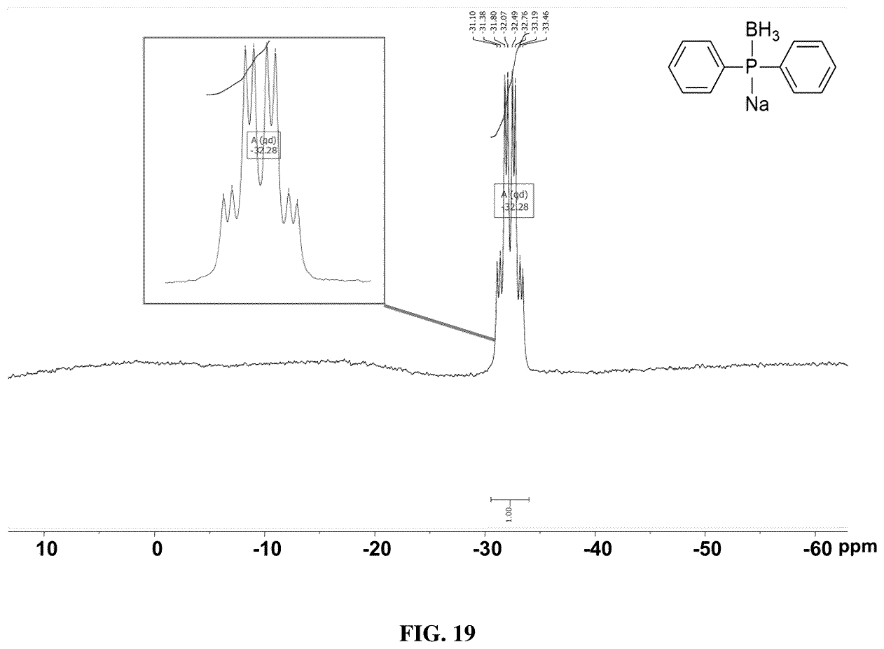

¹¹B NMR (128 MHz, THF-d₈) δ −32.28 (qd, J=89.5, 35.5 Hz). (see FIG. 19)

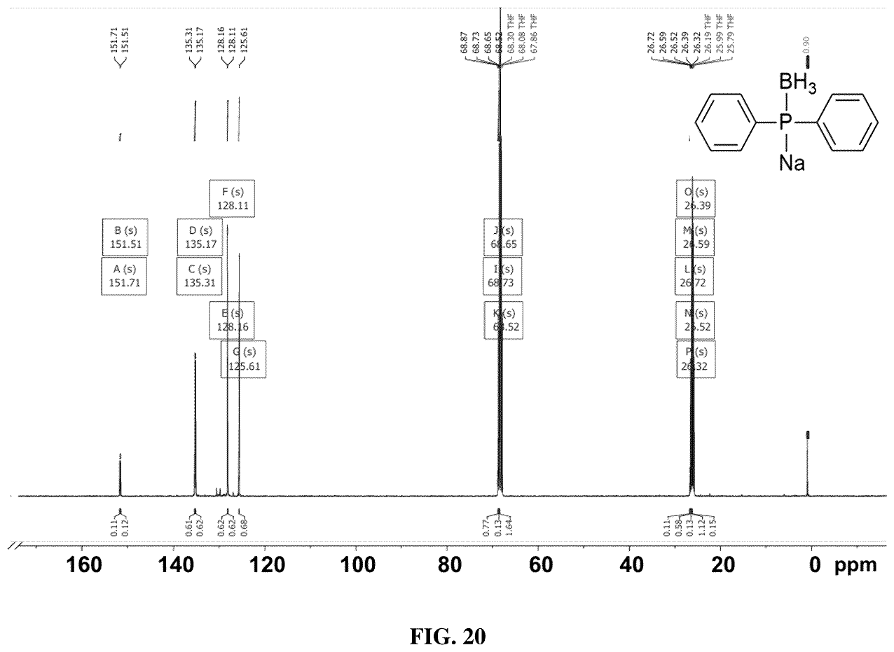

¹³C NMR (101 MHz, THF-d₈) δ 151.71, 151.51, 135.31, 135.17, 128.16, 128.11, 125.61, 68.73, 68.65, 68.52, 26.72, 26.59, 26.52, 26.39, 26.32. (see FIG. 20)

NMR analysis for Li(Ph₂PBH₃) salt provided the following results

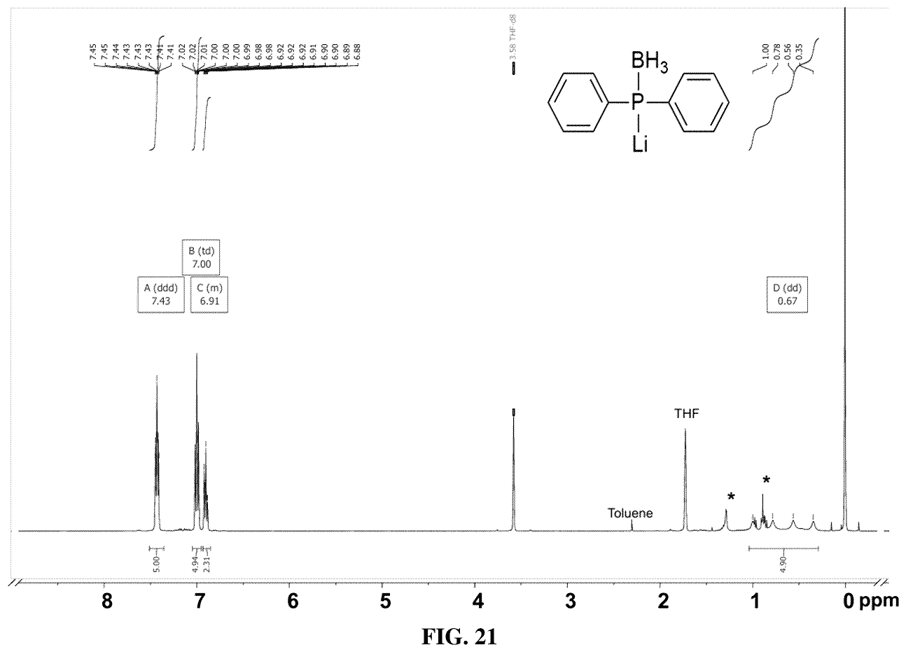

¹H NMR (400 MHz, THF-d₈) δ 7.43 (ddd, J=8.0, 6.1, 1.5 Hz, 5H), 7.00 (td, J=7.2, 1.3 Hz, 5H), 6.93-6.85 (m, 2H), 0.67 (dd, J=174.9, 86.6 Hz, 5H). (see FIG. 21)

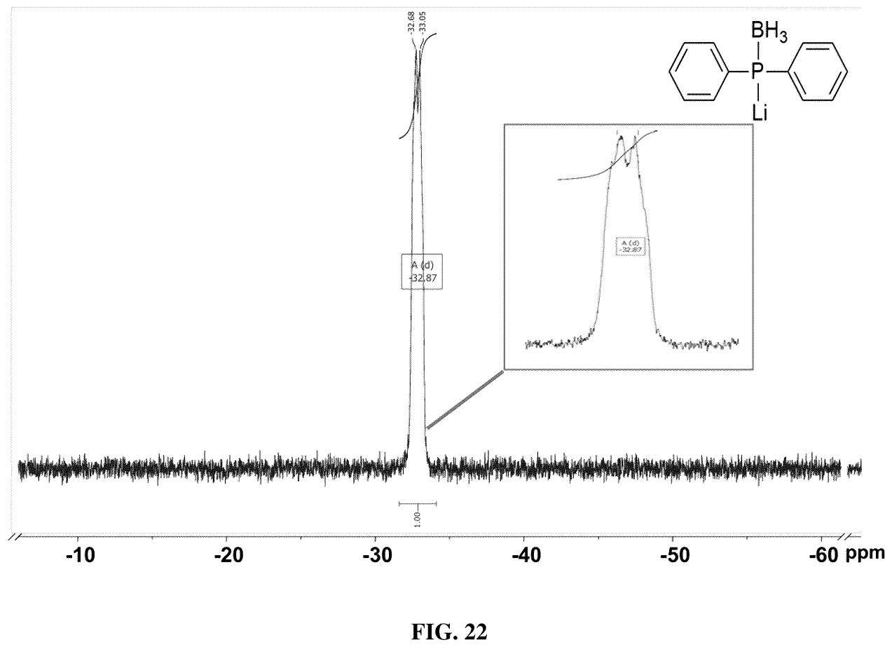

³¹P NMR (162 MHz, THF-d₈) δ −32.87 (d, J=60.7 Hz). (see FIG. 22)

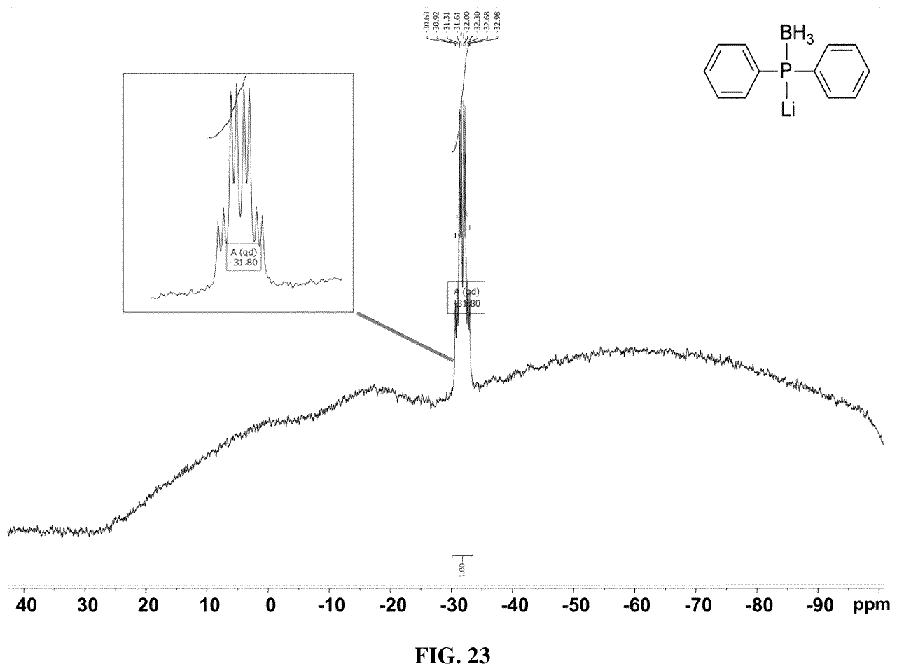

¹¹B NMR (128 MHz, THF-d₈) δ −32.09 (t, J=72.4 Hz). (see FIG. 23)

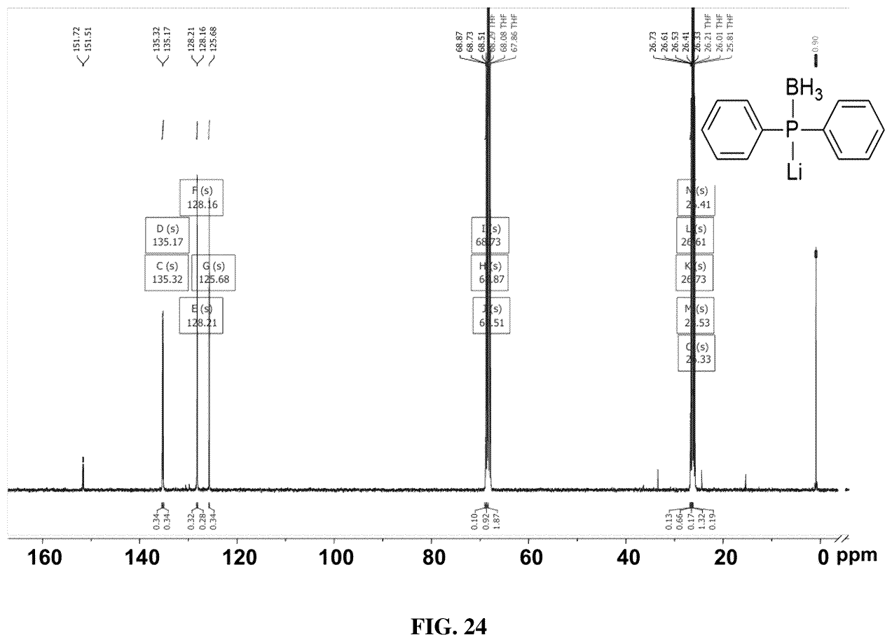

¹³C NMR (101 MHz, THF-d₈) δ 135.32, 135.17, 128.21, 128.16, 125.68, 68.87, 68.73, 68.51, 26.73, 26.61, 26.53, 26.41, 26.33. (see FIG. 24)

NMR analysis for K(Ph₂PBH₃) salt provided the following results

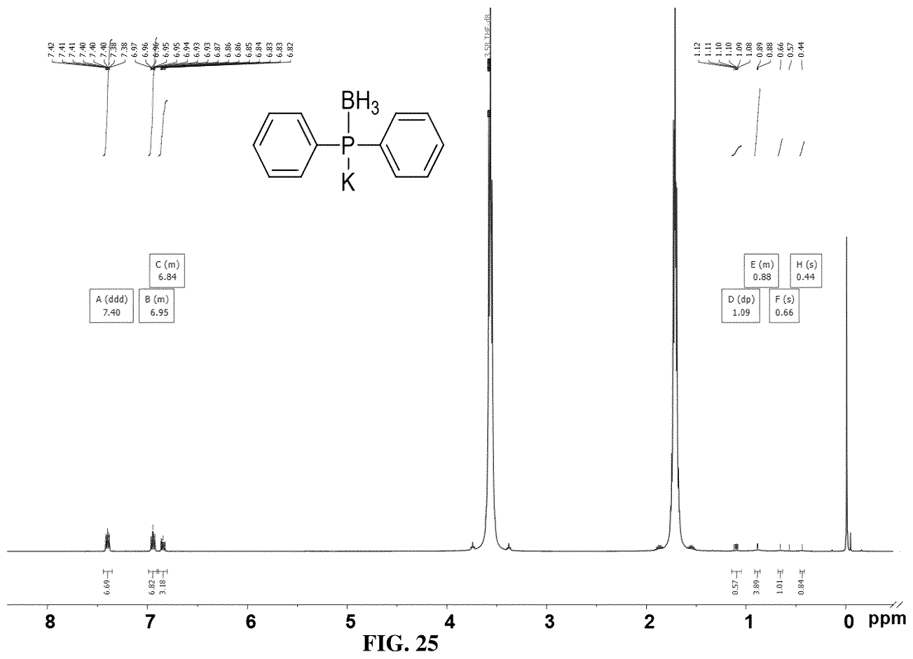

¹H NMR (400 MHz, THF-d₈) δ 7.40 (ddd, J=7.8, 5.9, 1.4 Hz, 7H), 6.99-6.90 (m, 7H), 6.89-6.80 (m, 3H), 1.09 (dp, J=9.1, 4.1, 3.0 Hz, 1H), 0.91-0.86 (m, 4H), 0.66 (s, 1H), 0.44 (s, 1H). (see FIG. 25)

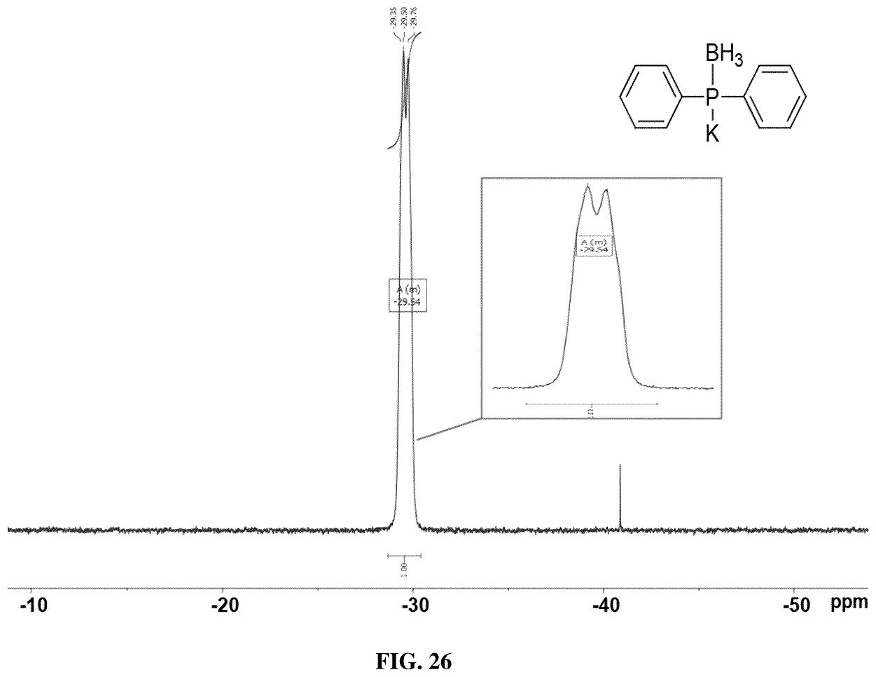

³¹P NMR (162 MHz, THF-d₈) δ −28.69-−30.43 (m). (see FIG. 26)

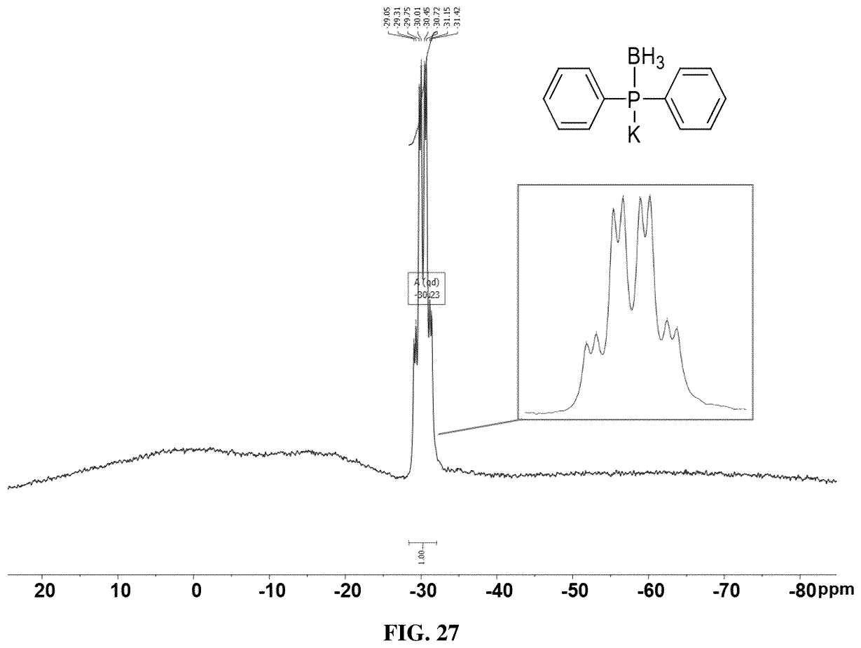

¹¹B NMR (128 MHz, THF-d₈) δ −30.23 (qd, J=90.5, 90.1, 33.8 Hz). (see FIG. 27)

¹³C NMR (101 MHz, THF-d₈) δ 149.19, 148.98, 132.58, 132.44, 125.50, 125.45, 122.90, 65.82, 65.60, 65.38, 65.16, 23.90, 23.70, 23.50, 23.30, 23.10, −1.76. (see FIG. 28)

### Example 11: Synthesis of Anionic Diphenyl Phosphine Borane CO₂ Adducts

A typical one pot procedure for preparing the CO₂ adducts was adding a solution of Ph₂PHBH₃ (5.314 mmol, in 4 ml of THF) to solution of MHMDs (5.314 mmol, in 4 ml of THF) dropwisely. An aliquot of the resulting was analyzed with the nmr to confirm the complete formation of M(Ph₂PBH₃) salt. The bulk solution was then saturated with CO₂ gas under standard atmospheric pressure and temperature. The product was then crushed out with hexanes resulting in a whitish precipitate. Yield 98.5%

NMR analysis for Na(Ph₂PBH₃)—CO₂ Adduct provided the following results

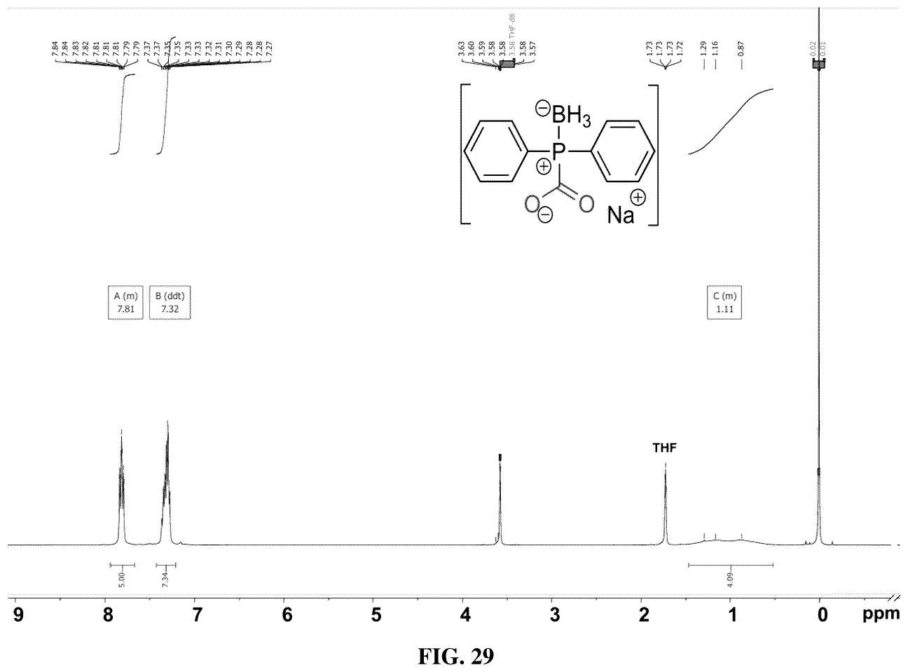

¹H NMR (400 MHz, THF-d₈) δ 7.94-7.67 (m, 5H), 7.32 (ddt, J=16.2, 8.7, 4.1 Hz, 7H), 1.47-0.52 (m, 4H). (see FIG. 29)

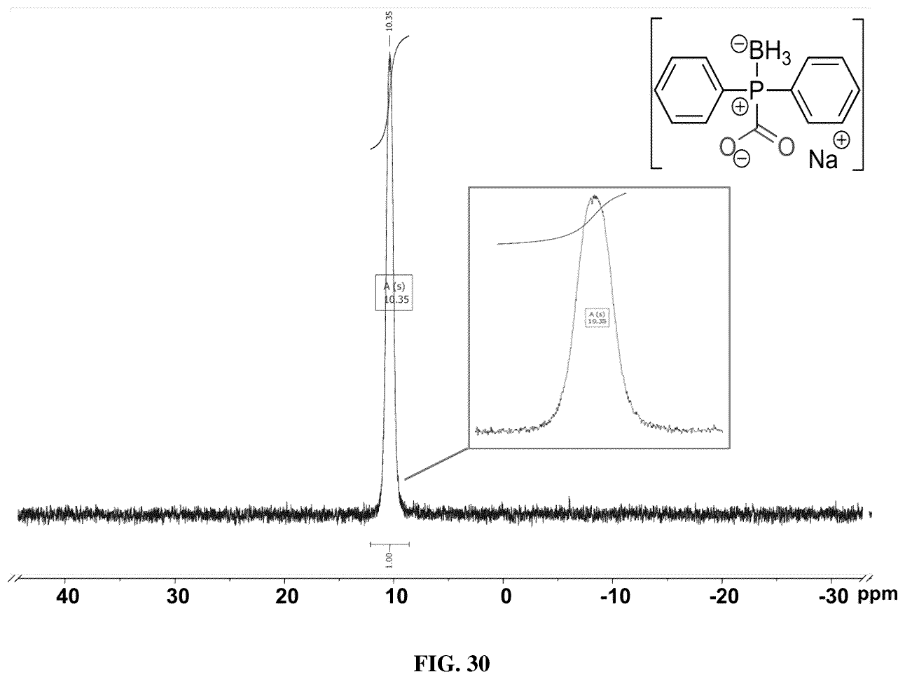

³¹P NMR (162 MHz, THF-d₈) δ 10.35. (see FIG. 30)

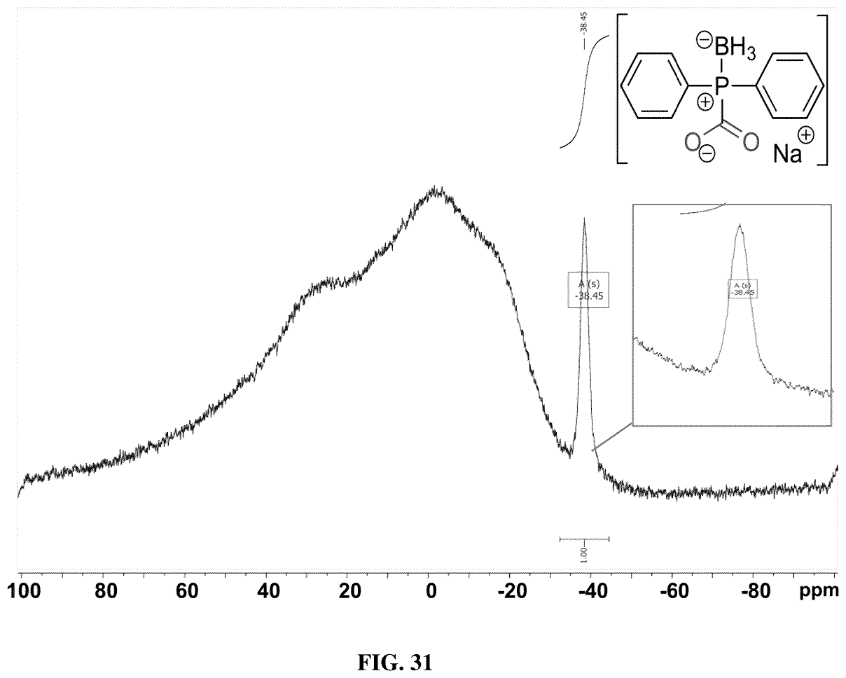

¹¹B NMR (128 MHz, THF-d₈) δ −38.45. (see FIG. 31)

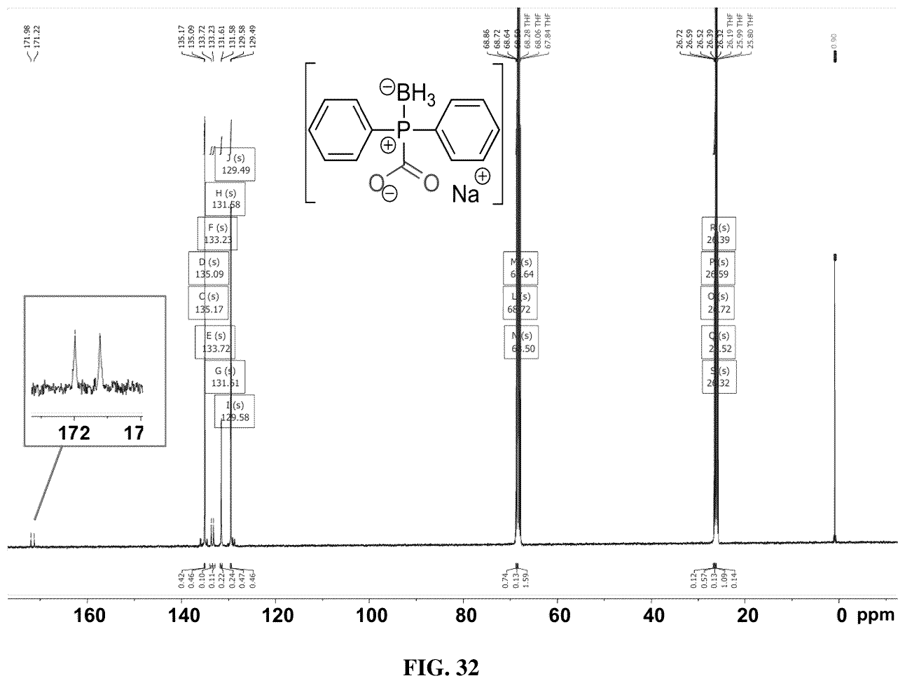

¹³C NMR (101 MHz, THF-d₈) δ 171.98, 171.22, 135.17, 135.09, 133.72, 133.23, 131.61, 131.58, 129.58, 129.49, 68.72, 68.64, 68.50, 26.72, 26.59, 26.52, 26.39, 26.32. (see FIG. 32)

NMR analysis for Li(Ph₂PBH₃)—CO₂ Adduct provided the following results

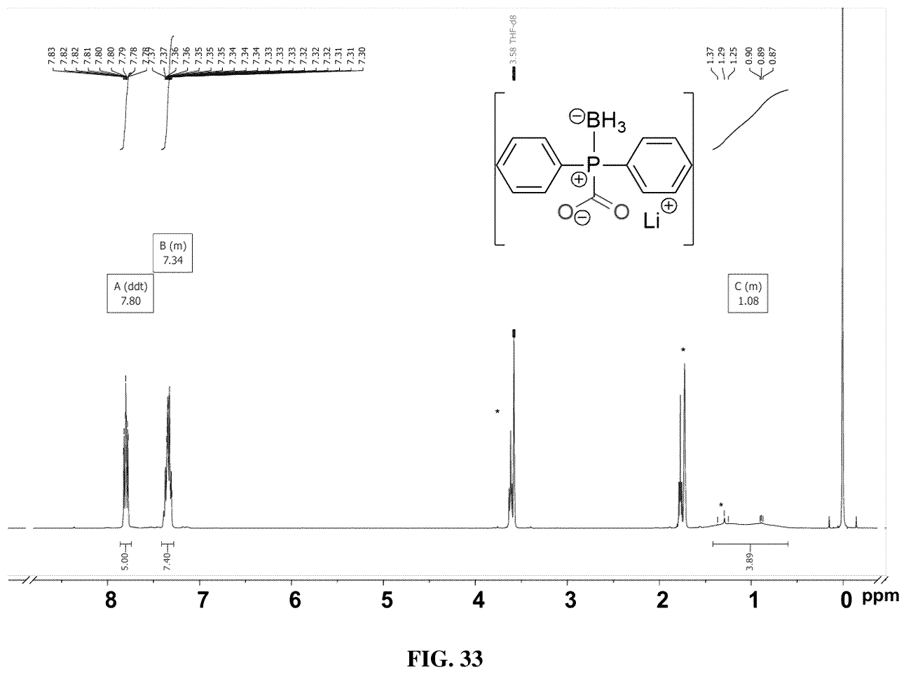

¹H NMR (400 MHz, THF-d₈) δ 7.80 (ddt, J=9.9, 6.7, 1.6 Hz, 5H), 7.41-7.28 (m, 7H), 1.42-0.60 (m, 4H). (see FIG. 33)

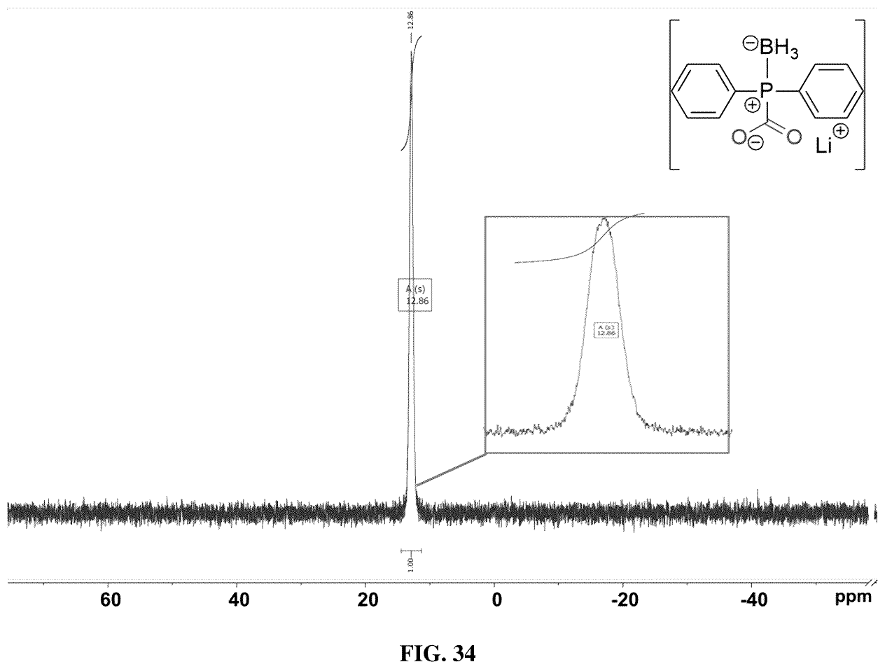

³¹P NMR (162 MHz, THF-d₈) δ 12.86. (see FIG. 34)

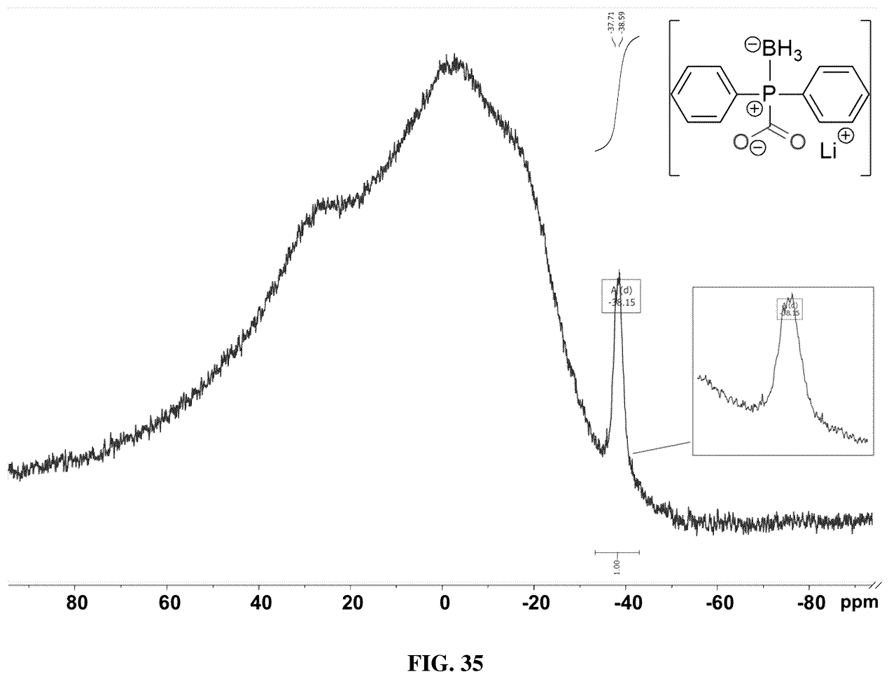

¹¹B NMR (128 MHz, THF-d₈) δ −38.15 (d, J=113.1 Hz) (see FIG. 35).

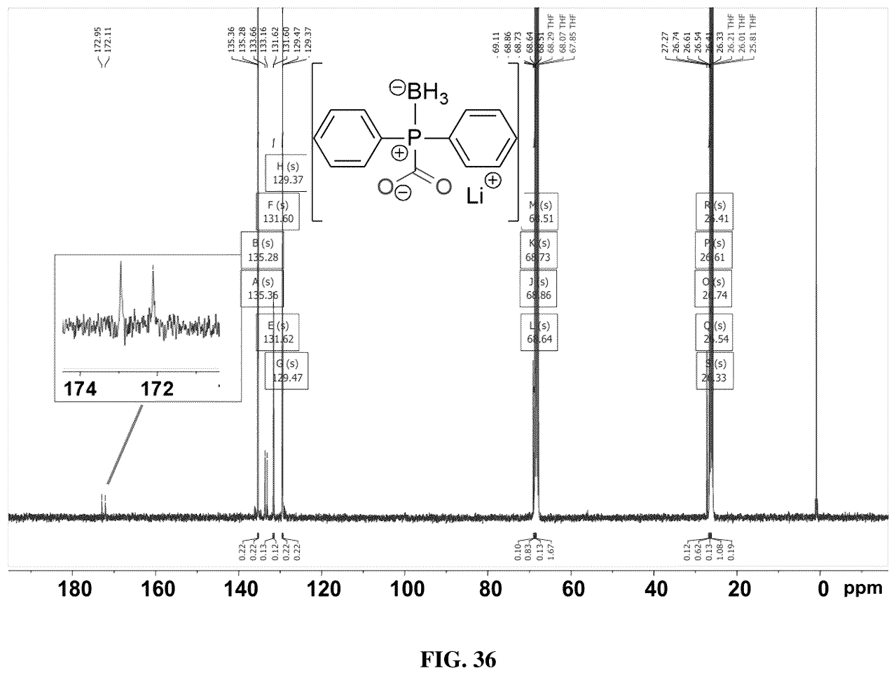

¹³C NMR (101 MHz, THF-d₈) δ 172.55, 172.33, 135.36, 135.28, 131.62, 131.60, 129.47, 129.37, 68.86, 68.73, 68.64, 68.51, 26.74, 26.61, 26.54, 26.41, 26.33. (see FIG. 36)

NMR analysis for K(Ph₂PBH₃)—CO₂ Adduct provided the following results

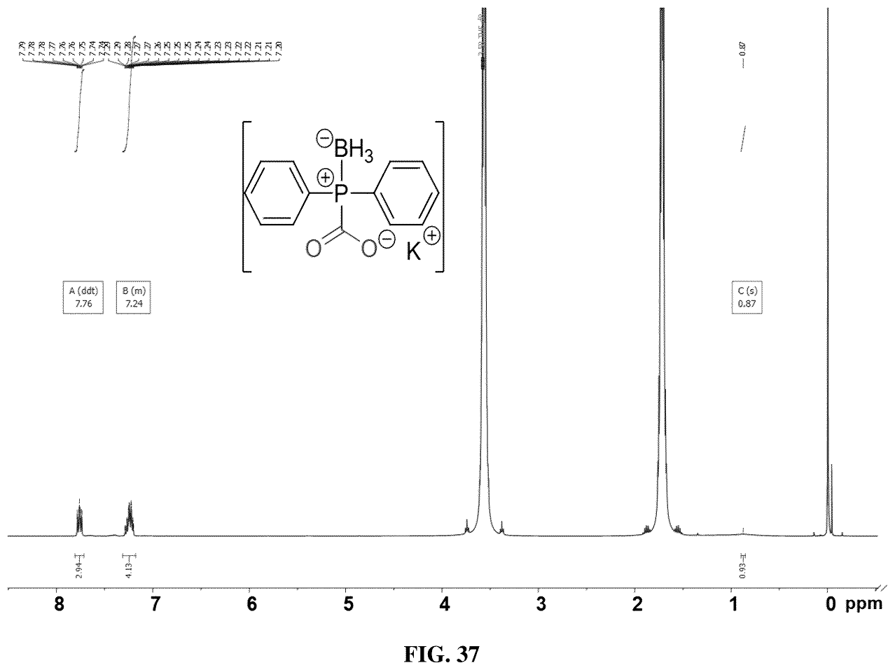

¹H NMR (400 MHz, THF-d₈) δ 7.76 (ddt, J=9.8, 6.6, 1.6 Hz, 3H), 7.31-7.18 (m, 4H), 0.87 (s, 1H). (see FIG. 37)

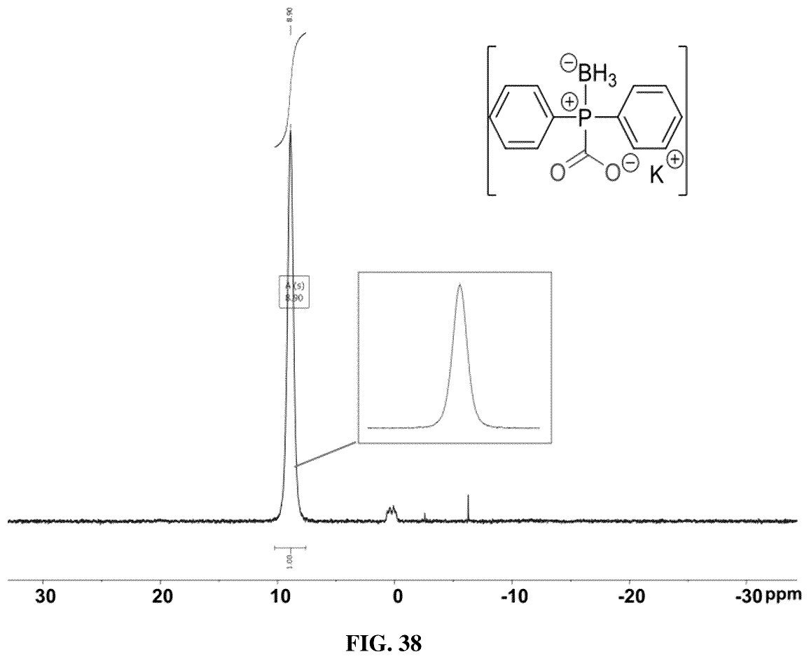

³¹P NMR (162 MHz, THF-d₈) δ 8.90. (see FIG. 38)

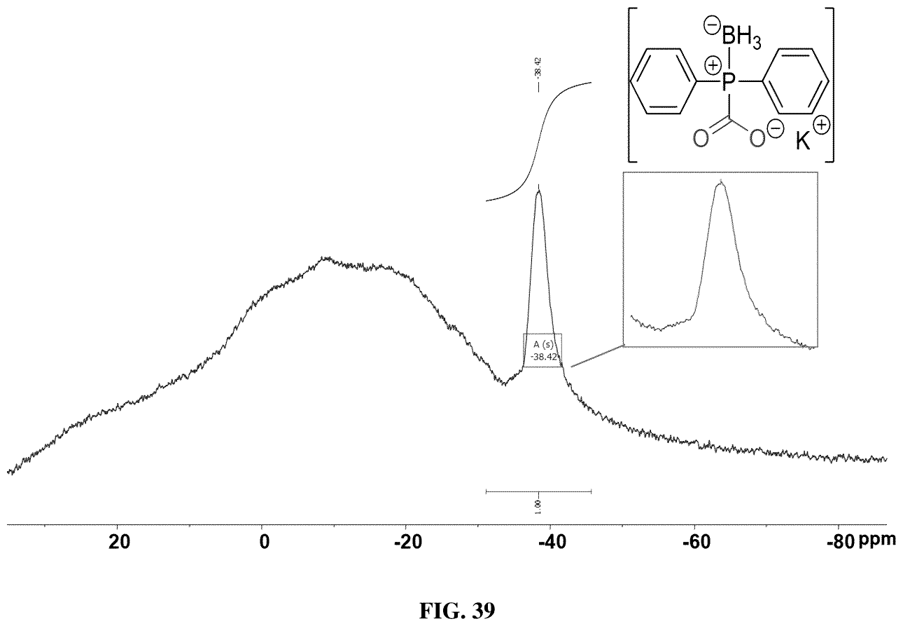

¹¹B NMR (128 MHz, THF-d₈) δ −38.42. (see FIG. 39)

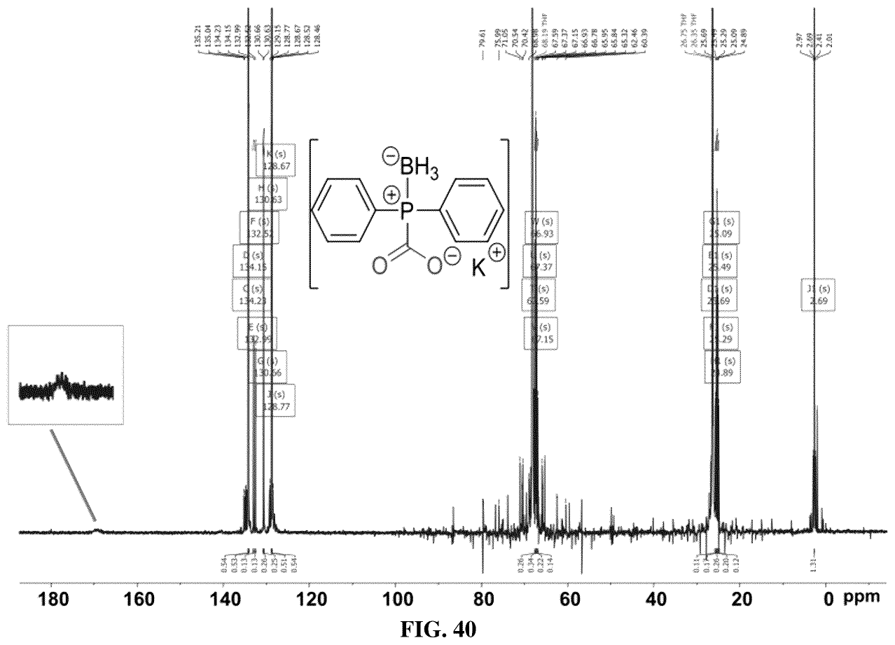

¹³C NMR (101 MHz, THF-d₈) δ 134.23, 134.15, 132.99, 132.52, 130.66, 130.63, 128.77, 128.67, 67.59, 67.37, 67.15, 66.93, 25.69, 25.49, 25.29, 25.09, 24.89, 2.69. (see FIG. 40)

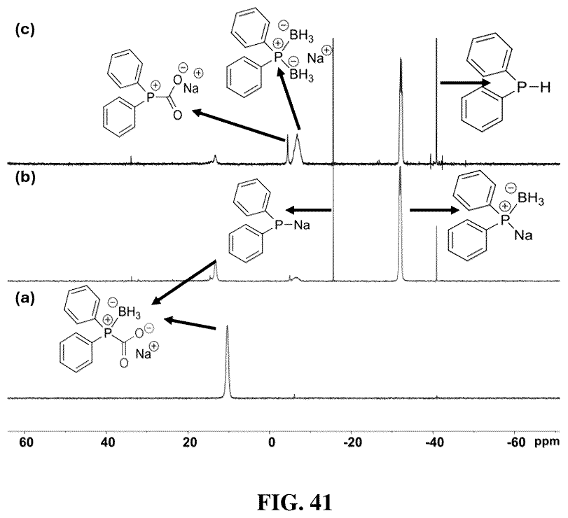

Stability of Exposed NaPh₂PBH₃—CO₂ Adduct to Atmosphere provided the following results are illustrated in FIG. 41 which shows ³¹P NMR of (a) NaPh₂PBH₃—CO₂ (without exposure) (b) NaPh₂PBH₃—CO₂ (1 hr exposure to atmosphere) (c) NaPh₂PBH₃—CO₂ (exposed sample stored for 3 days)

### Example 12: Synthesis of Diethylphosphine Borane Complex (Et₂PHBH₃)

The Synthesis of Diethylphosphine Borane Complex (Et₂PHBH₃) was performed with similar procedure as described in the diphenyl case.

NMR analysis for Et₂PHBH₃ provided the following results

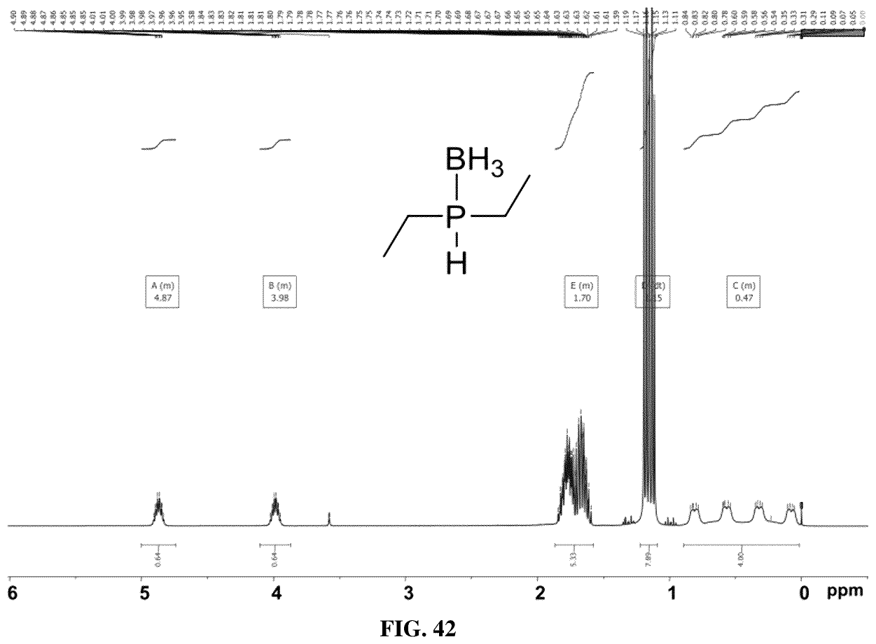

¹H NMR (400 MHz, THF-d₈) δ 5.00-4.74 (m, 1H), 4.10-3.87 (m, 1H), 1.87-1.57 (m, 5H), 1.15 (dt, J=16.8, 7.7 Hz, 8H), 0.89-0.01 (m, 4H). (see FIG. 42)

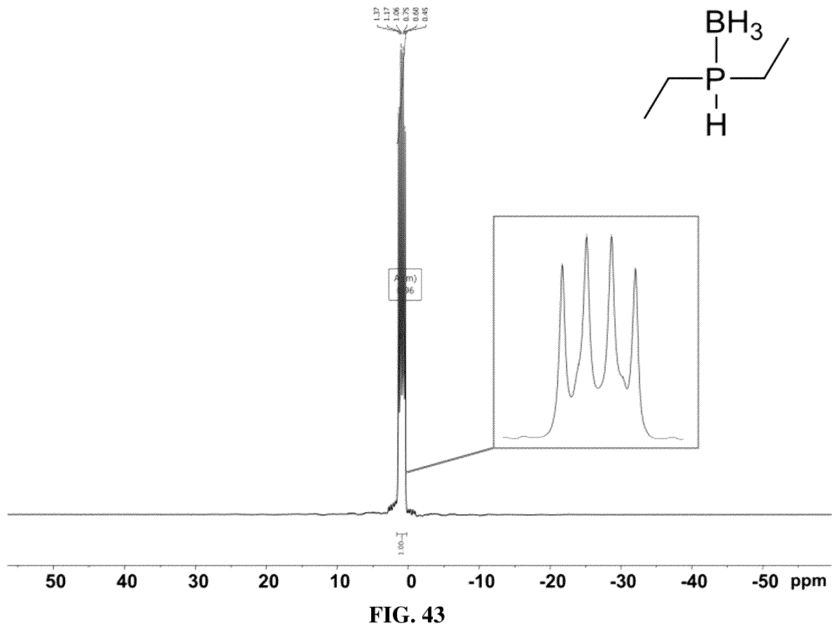

³¹P NMR (162 MHz, THF-d₈) δ 1.61-0.21 (m). (see FIG. 43)

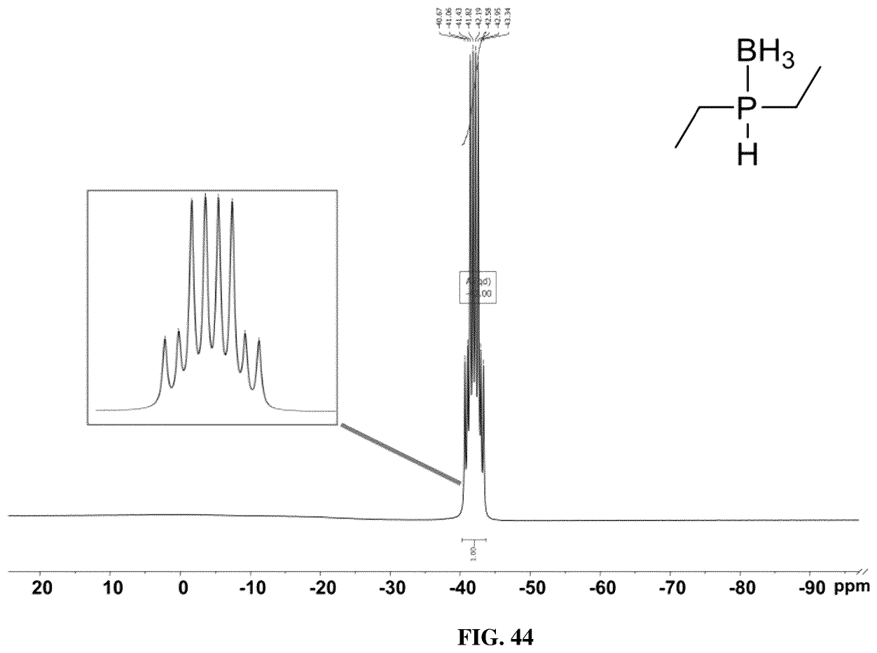

¹¹B NMR (128 MHz, THF-d₈) δ −42.00 (qd, J=97.8, 50.2 Hz). (see FIG. 44)

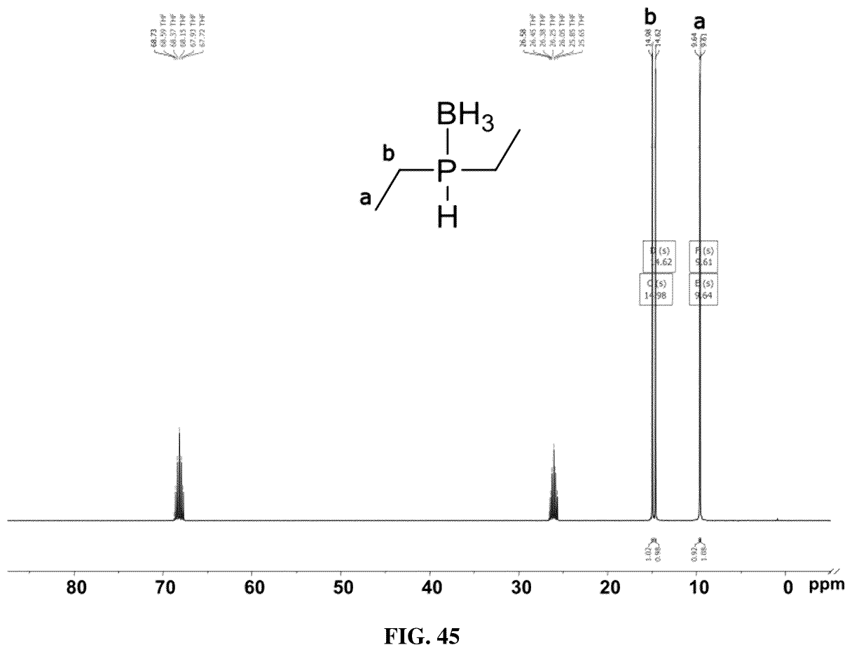

¹³C NMR (101 MHz, THF-d₈) δ 14.98, 14.62, 9.64, 9.61. (see FIG. 45)

NMR analysis for Li(Et₂PBH₃) provided the following results

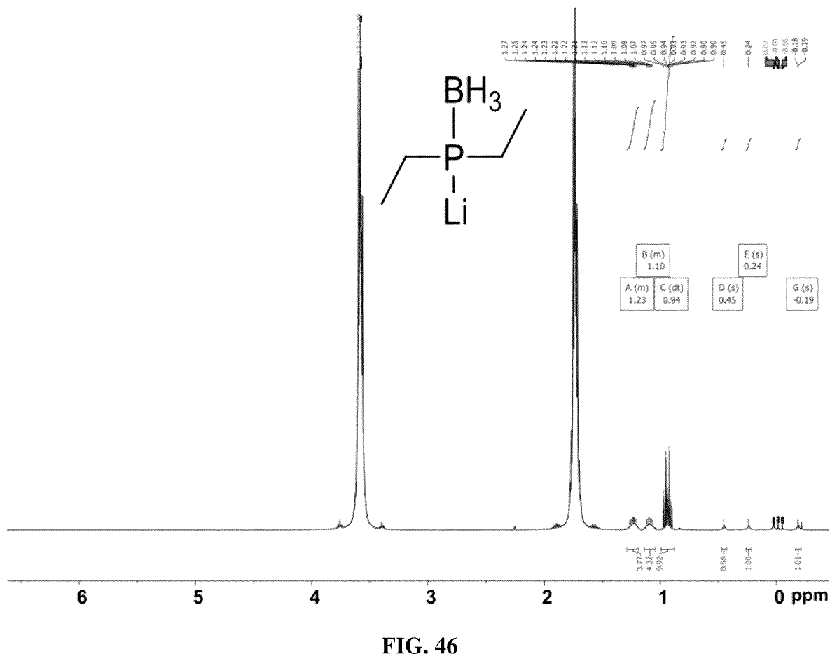

¹H NMR (400 MHz, THF-d₈) δ 1.29-1.19 (m, 4H), 1.14-1.05 (m, 4H), 0.94 (dt, J=12.6, 7.4 Hz, 10H), 0.45 (s, 1H), 0.24 (s, 1H), −0.19 (s, 1H). (see FIG. 46)

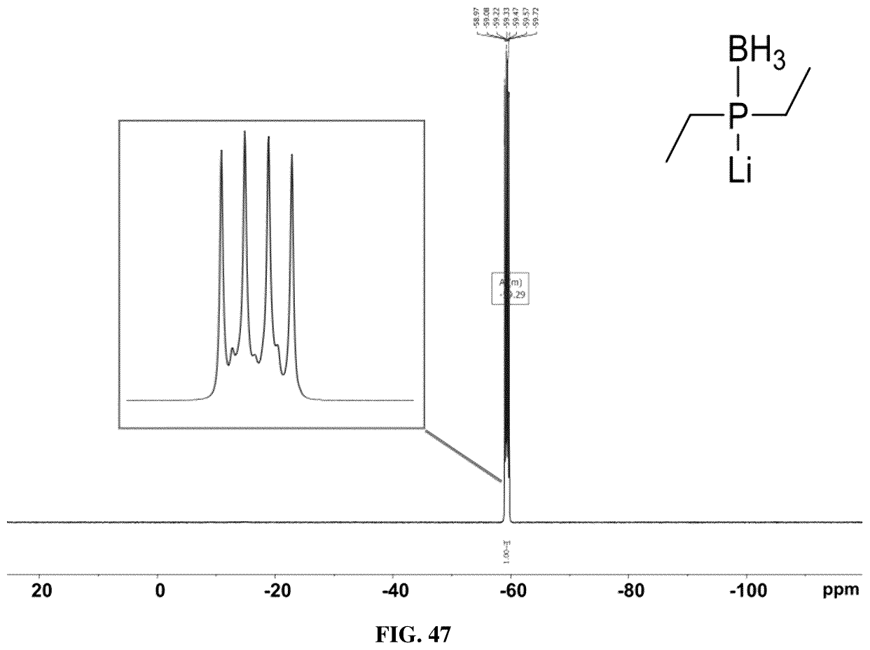

³¹P NMR (162 MHz, THF-d₈) δ −58.85-−59.83 (m). (see FIG. 47)

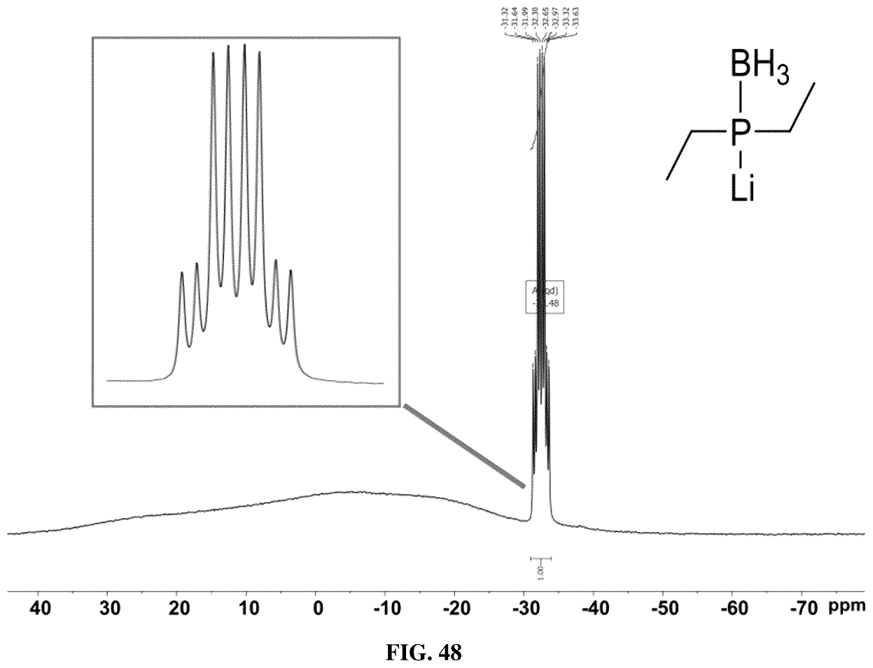

¹¹B NMR (128 MHz, THF-d₈) δ −32.48 (qd, J=85.2, 40.6 Hz). (see FIG. 48)

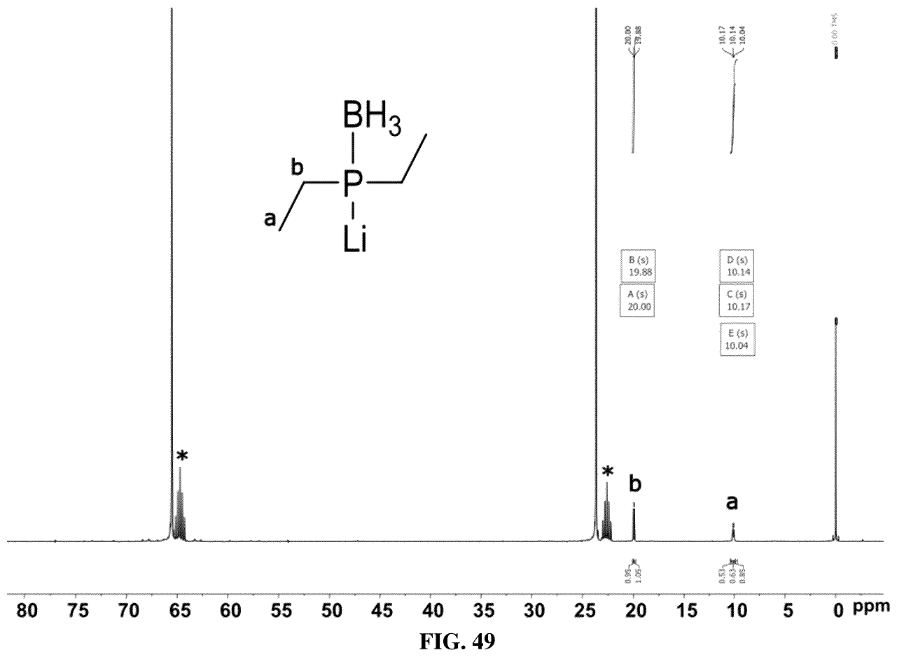

¹³C NMR (101 MHz, THF-d₈) δ 20.90, 20.78, 11.07, 11.04, 10.94. (see FIG. 49)

NMR analysis for Li(Et₂PBH₃)—CO₂ Adduct provided the following results

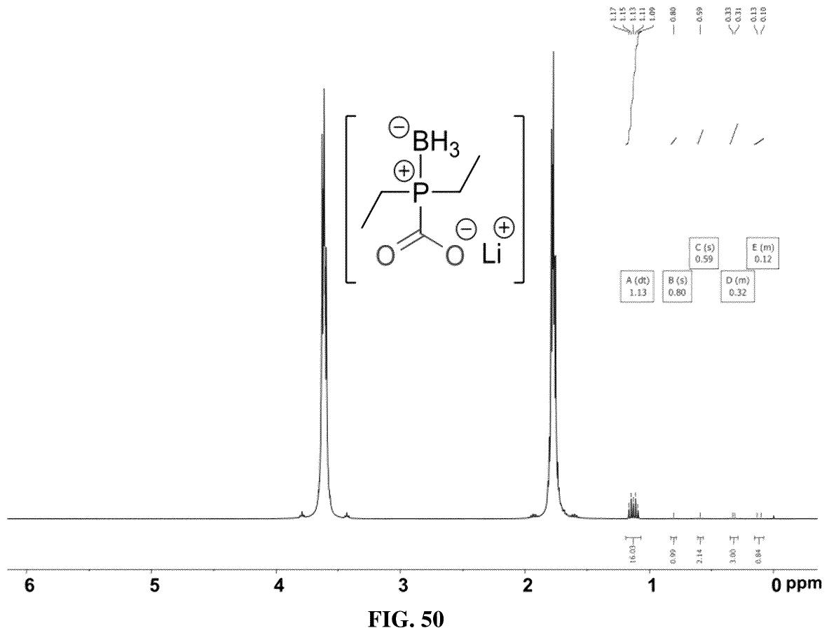

¹H NMR (400 MHz, THF-d₈) δ 1.13 (dt, J=15.0, 7.6 Hz, 16H), 0.80 (s, 1H), 0.59 (s, 2H), 0.35-0.29 (m, 3H), 0.15-0.08 (m, 1H). (see FIG. 50)

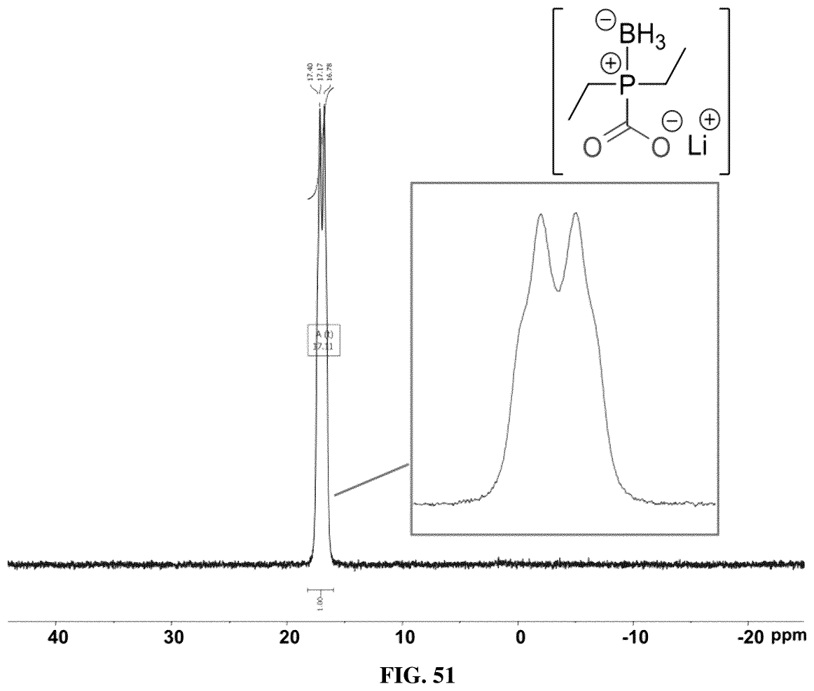

³¹P NMR (162 MHz, THF-d₈) δ 17.11 (t, J=50.1 Hz). (see FIG. 51)

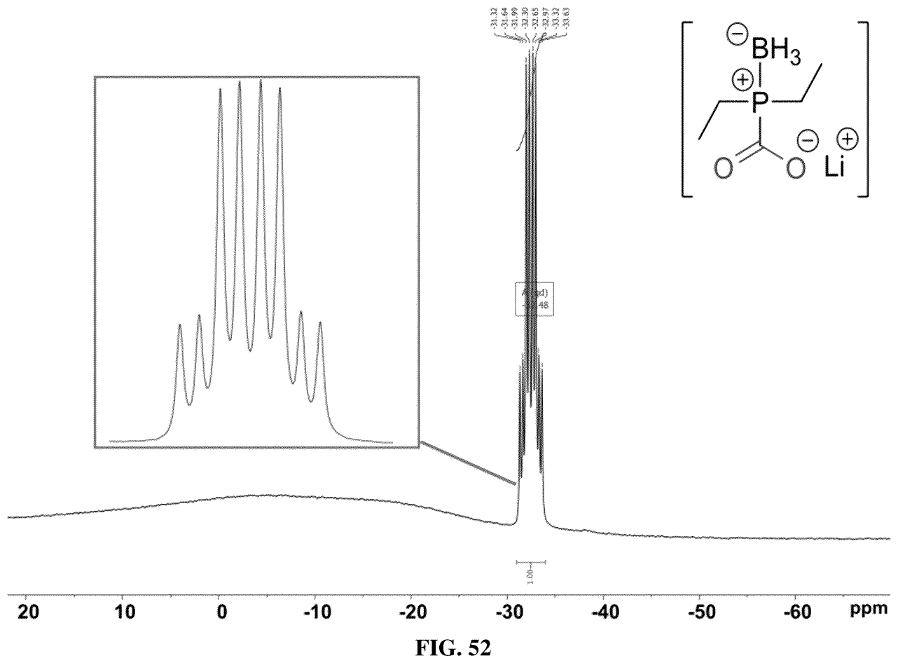

¹¹B NMR (128 MHz, THF-d₈) δ −32.48 (qd, J=85.2, 40.6 Hz). (see FIG. 52)

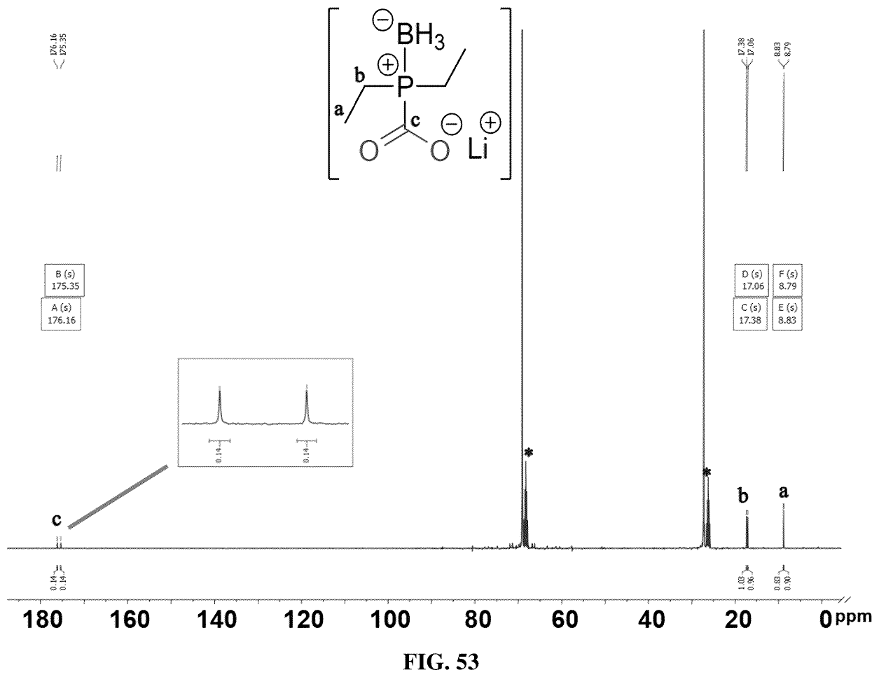

¹³C NMR (101 MHz, THF-d₈) δ 176.16, 175.35, 17.38, 17.06, 8.83, 8.79. (see FIG. 53)

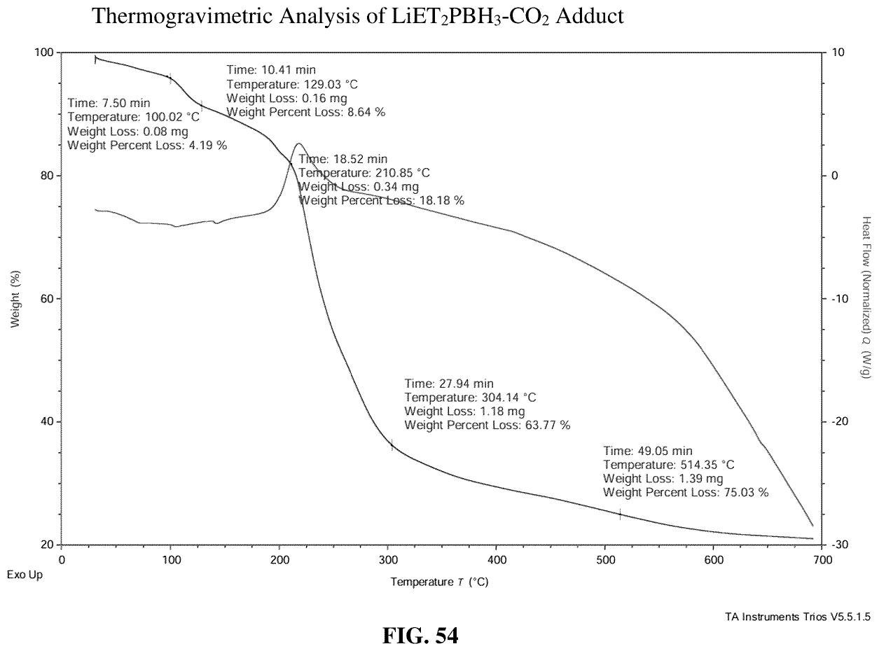

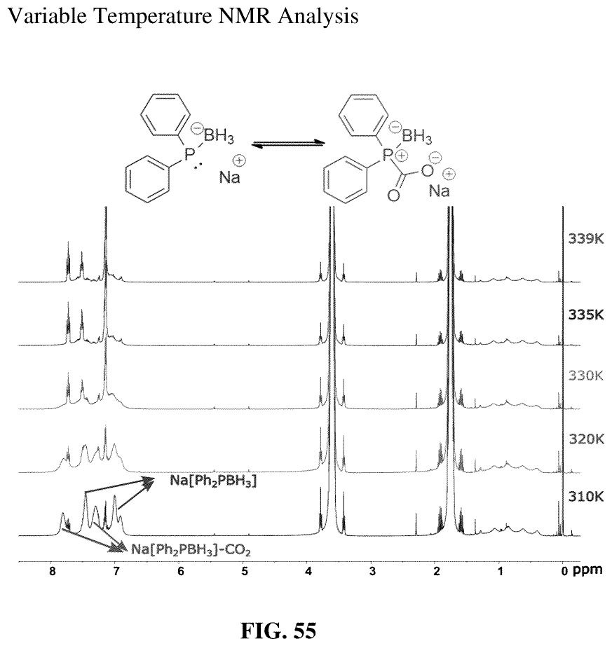

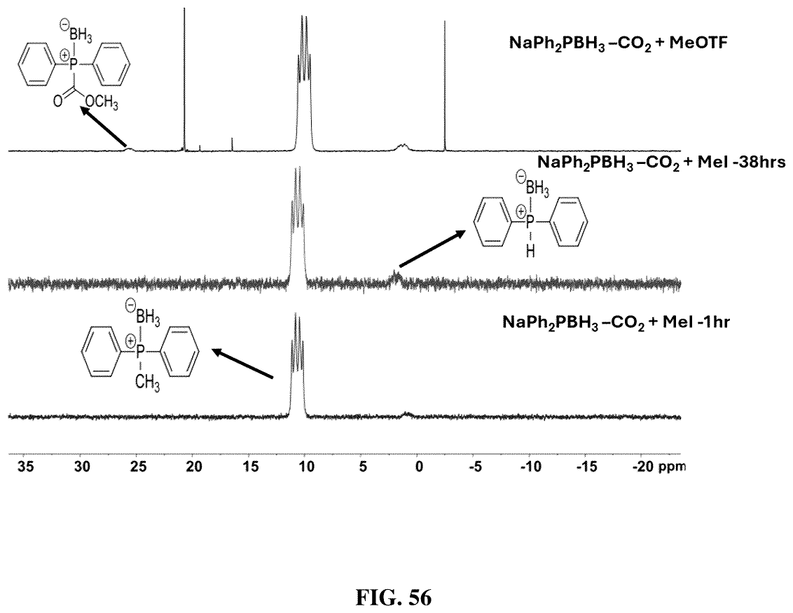

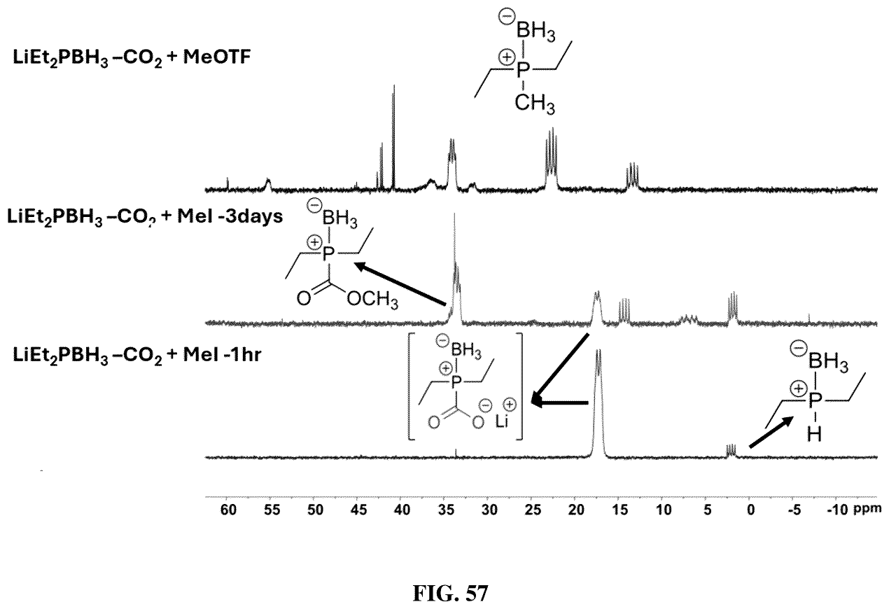

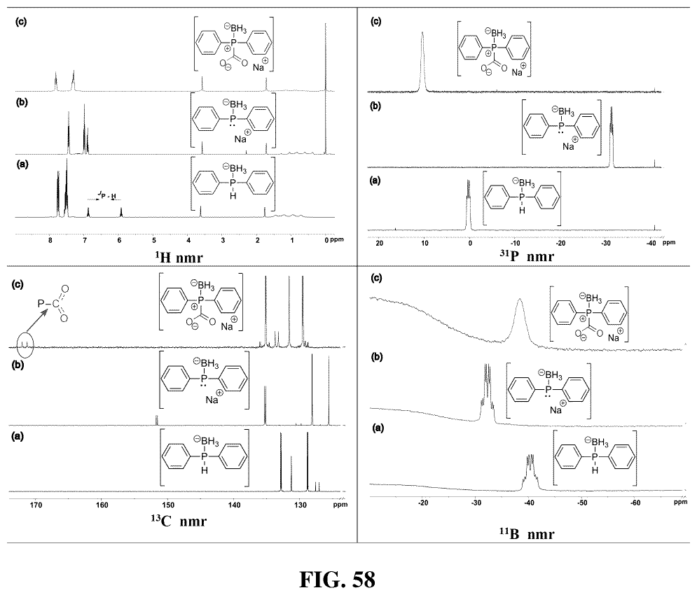

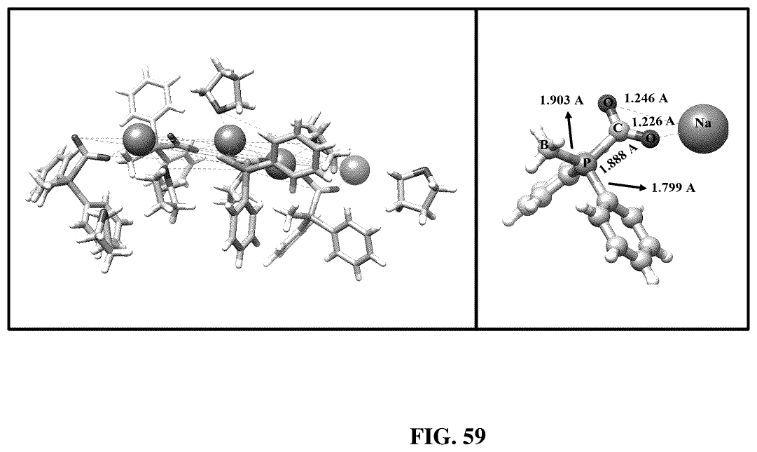

The thermogravimetric Analysis of LiET₂PBH₃—CO₂ Adduct provided the results
TGA of LiETPBH₃ CO₂ Adduct Variable Temperature NMR Analysis shown in FIG. 54;
VT NMR of NaPh₂PBH₃ and NaPh₂PBH₃—CO₂ Adduct in equilibrium shown in FIG. 55:
³¹P NMR of Attempted O-Alkylation of NaPh₂PBH₃—CO₂ Adduct shown in FIG. 56
³¹P NMR of Attempted O-Alkylation of LiEt₂PBH₃—CO₂ Adduct shown in FIG. 57.

### Example 13: Experimental Validation of DTF determination Diphenyl Phosphine Borane

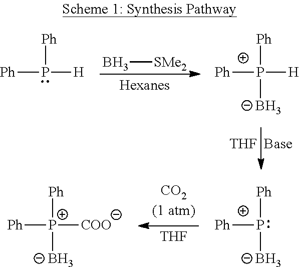

The scheme above describes a summary synthetic pathway of the disclosure. The procedure involves complexation of the borane moiety on the phosphorus atom to synthesize the parent phosphine borane, followed by deprotonation of the parent phosphine borane to create the anionic phosphine borane complex which was then subjected to a stream of CO₂ gas leading to the creation of the CO₂ adducts. The synthesized materials were further validated using Nuclear Magnetic Resonance (NMR) technique and Single Crystal X-Ray analysis as shown in FIG. 3 and FIG. 4 respectively. The chemical shifts and multiplicity of the NMR spectra of the synthesized material are reported below:

NMR Analysis of The Synthesized Diphenyl Phosphine Borane (Ph₂PHBH₃) provided the following result

¹H NMR (400 MHz, THF-d₈) δ 7.75-7.65 (m, 5H), 7.53-7.40 (m, 7H), 6.83 (q, J=7.1 Hz, 1H), 5.88 (q, J=7.1 Hz, 1H), 1.04 (dd, J=199.4, 94.5 Hz, 4H).

³¹P NMR (162 MHz, THF-d₈) δ 1.16-−0.79 (m).

¹¹B NMR (128 MHz, THF-d₈) δ −40.33 (qd, J=101.1, 100.0, 43.3 Hz).

¹³C NMR (101 MHz, THF-d₈) δ 132.88, 132.79, 131.29, 131.27, 128.87, 128.76, 66.22, 66.00, 24.15, 23.95.

NMR Analysis of The Synthesized Anionic Diphenyl Phosphine Borane (Na[Ph₂PBH₃]) provided the following

¹H NMR (400 MHz, THF-d₈) δ 7.44 (ddt, J=7.2, 6.0, 1.4 Hz, 5H), 6.99 (tq, J=6.7, 0.9 Hz, 5H), 6.93-6.85 (m, 2H), 0.72 (dd, J=176.9, 87.4 Hz, 4H).

³¹P NMR (162 MHz, THF-d₈) δ −30.69-−32.08 (m).

¹¹B NMR (128 MHz, THF-d₈) δ −32.28 (qd, J=89.5, 35.5 Hz).

¹³C NMR (101 MHz, THF-d₈) δ 151.71, 151.51, 135.31, 135.17, 128.16, 128.11, 125.61, 68.73, 68.65, 68.52, 26.72, 26.59, 26.52, 26.39, 26.32

NMR Analysis of The Synthesized Anionic Diphenyl Phosphine Borane—CO₂ Adducts (Na[Ph₂PBH₃]—CO₂) provided the following results

¹H NMR (400 MHz, THF-d₈) δ 7.94-7.67 (m, 5H), 7.32 (ddt, J=16.2, 8.7, 4.1 Hz, 7H), 1.47-0.52 (m, 4H).

³¹P NMR (162 MHz, THF-d₈) δ 10.35.

¹¹B NMR (128 MHz, THF-d₈) δ −38.45.

¹³C NMR (101 MHz, THF-d₈) δ 171.98, 171.22, 135.17, 135.09, 133.72, 133.23, 131.61, 131.58, 129.58, 129.49, 68.72, 68.64, 68.50, 26.72, 26.59, 26.52, 26.39, 26.32

The related spectra are illustrated in FIG. 1 EV: NMR Spectra of (a) Ph₂PHBH₃, (b) Na[Ph₂PBH₃] and (c) Na[Ph₂PBH₃]—CO₂

### Example 14: Single Crystal X-ray Diffraction Measurements of Na(Ph₂PBH₃)—CO₂ Adduct

Diffraction data were collected at 100 K on a Bruker D8 Advance Quest diffractometer with a graphite monochromator using Mo Kα radiation (λ=0.71073 Å). The frames were integrated with the Bruker SAINT software package using a narrow-frame algorithm. An empirical absorption correction using the Multi-Scan method SADABS was applied to the data. The structure was solved by direct methods using the Bruker SHELXTL Software Package, version 2019/1. All atoms except the H atoms were refined anisotropically. Hydrogen atoms were calculated and placed in idealized positions. Hydrogen atoms were not able to be added to atom B3 as a consequence of twinning.

TABLE EV
Crystallographic information for 1.

| | 1 |
|---|---|
| Formula | C₂₂8H₂₉₁B₁₂Na₁₂O₄₂P₁₂ |
| FW | 4480.84 |
| Crystal Class | Trigonal |
| Space Group | P3₁ |
| a (Å) | 14.062(6) |
| b (Å) | 14.062(6) |
| c (Å) | 35.554(10) |
| α (°) | 90 |
| β (°) | 90 |
| γ (°) | 120 |
| Z | 1 |
| Cell volume (Å³) | 6088.1(12) |
| Density (mg m⁻³) | 1.222 |
| μ (mm⁻¹) | 0.173 |
| Temp (K) | 101(2) |
| Total Reflns | 22737 |
| Rᵢₙₜ | 0.0405 |
| R₁^{a} | 0.0396 |
| wR₂^{a} | 0.0941 |
| GOF | 1.044 |

${}^{a} R 1 = \sum \frac{‖ {F}_{O} \vert - \vert {F}_{C} ‖}{\vert {F}_{O} \vert} ; wR 2 = {( \frac{\sum [ {w ( {F}_{O}^{2} - {F}_{C}^{2} )}^{2} ]}{\sum [ {w ( {F}_{O}^{2} )}^{2} ]} )}^{1 / 2}$

Single crystal analysis of the NaPh₂PBH₃—CO₂ adducts confirmed that CO₂ binds at the phosphorus atom with a P—C bond of 1.888 Å. The phosphine borane ligand (CO₂ molecule) is coordinated to the phosphorus atom and coordinated to the sodium through the oxygen molecules atoms of the CO₂ via some form of η² interaction. The coordination sphere of sodium is completed by approximately 1.5 molecules of the THF since it is evident from the unit cell that there are four molecules of the CO₂ adduct connected to four molecules of the sodium and 6 molecules of the THF. The P—C bond of the phenyl ring to the phosphorus is comparable to the P—C bond of the CO₂ molecule indicating a legit P—C bond formed between the phosphine borane and the CO₂ molecule. The P—B bond length of 1.903 Å, clearly suggest a dative covalent bond where the shared electron is donated by the P atom.

There results of the Single Crystal Analysis of Na(Ph₂PBH₃)—CO₂ Adduct are also illustrated in FIG. 2EV.

### Example 15 Device for CO₂ Capture

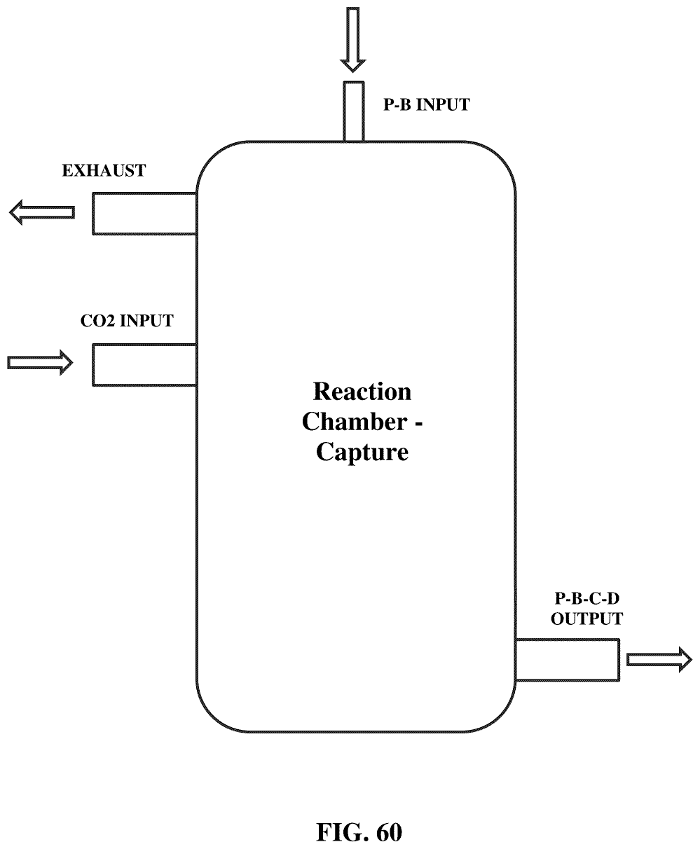

In some embodiments, the carbon dioxide capture is performed in a reaction chamber. An example capture reaction chamber is shown in FIG. 60.

The device includes a capture reaction chamber that houses the phosphine-borane compound in solid or liquid form, with an inlet providing the carbon dioxide infused fluid (such as ambient air, or the exhaust from an industrial process) to have the carbon dioxide, at least in part, removed. The fluid flows over or through the phosphine-borane compound in the chamber, producing a phosphine-borane-carbon-dioxide complex, which can be removed from the chamber through an outlet. The fluid, after having the carbon dioxide removed (in part or completely), can be expelled or collected from an exhaust outlet. Additional phosphine-borane compound can be introduced into the capture reaction chamber by an inlet. In some embodiments, the phosphine-borane compound can be in a liquid solution, where the fluid is bubbled through the solution. In some embodiments, the phosphine-borane compound can be in solid form, where the fluid is passed over through the solid. Examples of solid forms include powders, beads, meshes, honeycombs, and surfaces.

### Example 16 Device for Conversion Phosphine-Borane-Carbon-Dioxide Complex

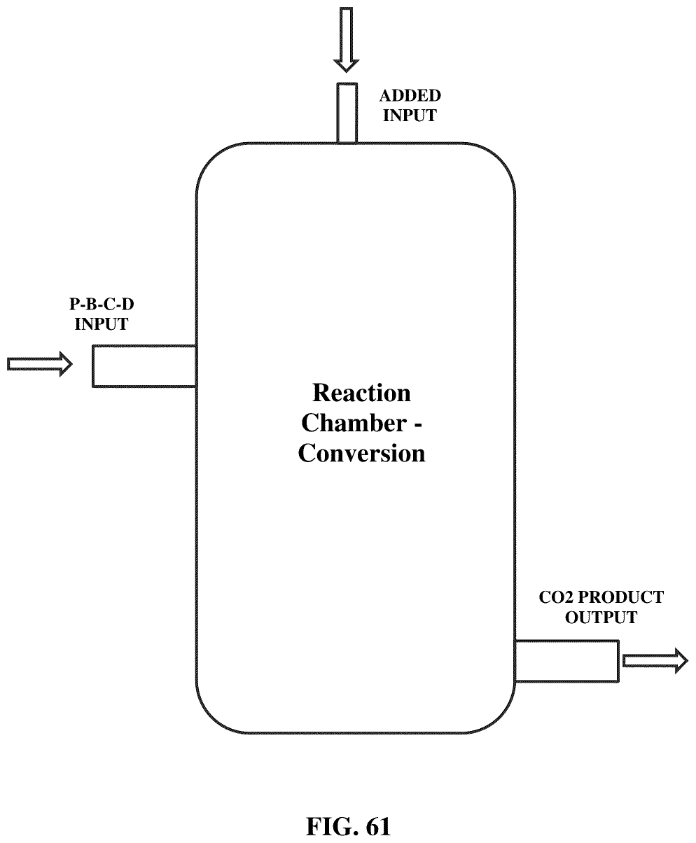

In some embodiments, the phosphine-borane-carbon-dioxide complex (which can possibly have been produced from a capture reaction chamber) can be processed in a “conversion” reaction chamber to further process the complex into a carbon/oxygen based product (such as carbon dioxide gas, ethanol, formic acid, ethylene, and other high order carbon based products). An example conversion reaction chamber is shown in FIG. 61.

The device includes a conversion reaction chamber that includes a means to convert the phosphine-borane-carbon-dioxide complex into the product. Examples of means to do this include 1) using a metal hydride or borohydride, 2) using an alkylating agent in a solvent, 3) applying a voltage across electrodes, 4) use of bio-reactive yeast, fungus, or bacterium. Alternative methods comprise operating temperature and pressure for releasing the CO₂. The phosphine-borane-carbon-dioxide complex is input to the conversion reaction chamber by an inlet, and the product is removed from the chamber by an outlet. The carbon dioxide based product can then be sent to a further processing system to convert it to a further product, such as one that would be useful in commerce (e.g. plastics, feedstock, electrolyte additives).

The device includes a conversion reaction chamber that includes reducing agents to convert the phosphine-borane-carbon-dioxide complex into the phosphine-borane-carbon-dioxide complex of Formula V.

The phosphine-borane-carbon-dioxide complex is input to the conversion reaction chamber by an inlet, and the phosphine-borane-carbon-dioxide complex of Formula V is removed from the chamber by corresponding outlets. The phosphine-borane-carbon-dioxide complex of Formula V can then be sent to a further processing system to convert it to a further product, such as one that would be useful in commerce. Exemplary products comprise plastics, ethanol, formate, methanol, formic acid, acetic acid and methane gasoline, ethylene, and other higher order C-products identifiable by a skilled person upon reading of the present disclosure.

### Example 17 Device for Conversion of Carbon Dioxide a Carbon Dioxide Reduction Product

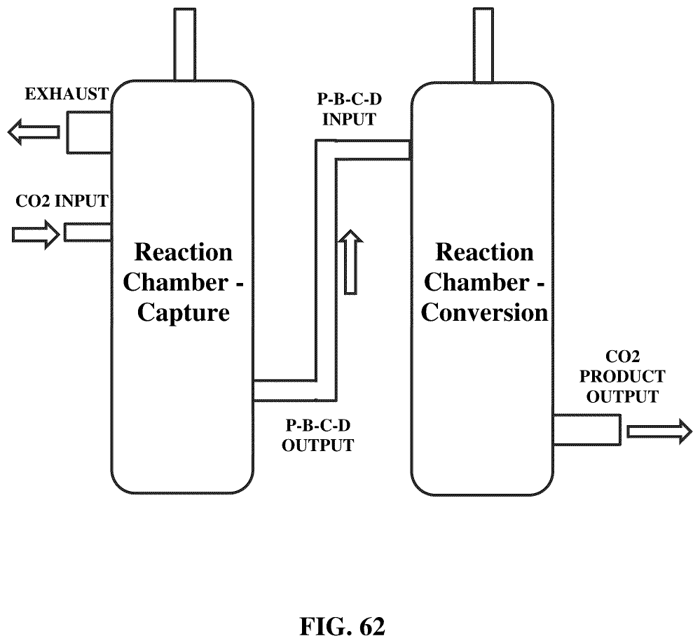

In some embodiments, the capture reaction chamber and the conversion reaction chambers can be connected to provide a system for utilizing carbon dioxide taken from a source. An example is shown in FIG. 62.

The phosphine-borane-carbon-dioxide complex outlet of the capture chamber is connected to the phosphine-borane-carbon-dioxide complex inlet of the conversion chamber. Additional chambers/tanks can be included in the system. For example, a storage tank of the phosphine-borane compound can be connected to the phosphine-borane compound inlet to store extra phosphine-borane compound for the capture chamber. As mentioned above, the product output can be sent to a further chamber for processing of the product into a further product, e.g. plastics, for a recycling of the carbon dioxide into useful products.

### Example 18 Device-CO₂ Air Scrubber

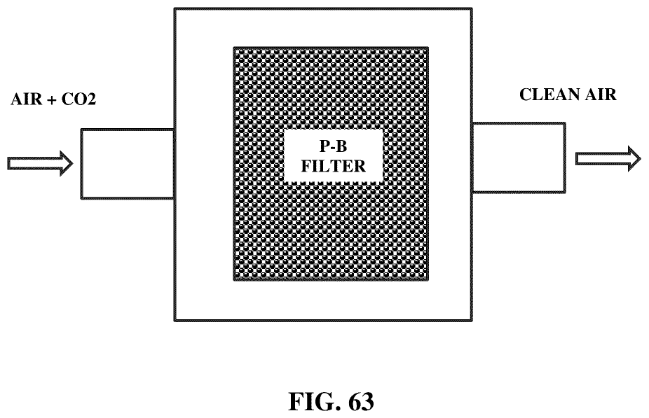

In some embodiments, carbon dioxide can be “scrubbed” from air (or other fluid) by a device that contains the phosphine-borane compound as a filter. An example of this device is shown in FIG. 63.

In the example, air containing carbon dioxide is input to the device in an inlet, passed over the filter (comprising the phosphine-borane compound) and expelled from an outlet as “cleaned” air (having a lower percentage of carbon dioxide). The filter can be a structure that maximizes the surface area of the phosphine-borane compound to the incoming air, such as a 3D honeycomb structure, a mesh, packed beads, layered surfaces, etc. The reaction changes the phosphine-borane compound into a phosphine-borane-carbon-dioxide complex. In some embodiments, the filter is replaceable or disposable from within the device as it is “used up”.

The examples set forth above are provided to give those of ordinary skill in the art a complete disclosure and description of how to make and use the embodiments of the phosphorous-boron bonded compounds, materials, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles including related supplemental and/or supporting information sections, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art, and that such modifications and variations are considered to be within the scope of this disclosure as defined by the appended claims.

It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

The term “alkyl” as used herein refers to a linear, branched, or cyclic saturated hydrocarbon group typically although not necessarily containing 1 to about 15 carbon atoms, or 1 to about 6 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 15 carbon atoms. The term “cycloalkyl” intends a cyclic alkyl group, typically having 4 to 8, or 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “heteroatom-containing” as in a “heteroatom-containing alky group” refers to an alkyl group in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, and additional substituents identifiable by a skilled person.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Aryl groups can contain 5 to 24 carbon atoms, or aryl groups contain 5 to 14 carbon atoms. Exemplary aryl groups contain one aromatic ring or two fused or linked aromatic rings, e.g., phenyl, naphthyl, biphenyl, diphenyl ether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The terms “cyclic”, “cyclo-”, and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic or polycyclic.

The term “isomers” as used refers to heterocyclic aromatic groups that have the same core molecular but may differ in atomic connectivity and/or location of unsaturation and is meant to include all possible structural variants. For example, as shown below, “pyrrole isomers” refers to all possible substituted variants of 1H-pyrrole and 2H-pyrrole; “indole isomers” refers to all possible substituted variants of 3H-indole, 1H-indole and 2H-isoindole, and so on:

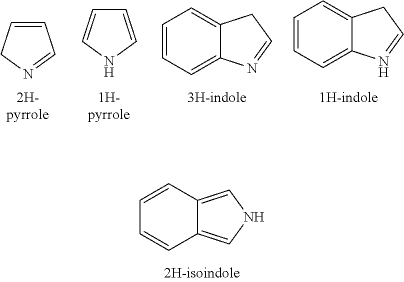

Likewise, as shown below, “triazole isomers” refers to all possible substituted variants of 1,2,4-triazole and 1,2,3-triazole; “oxadiazole isomers” refers to all possible substituted variants of 1,2,5-oxadiazole and 1,2,3-oxadiazole, and so on:

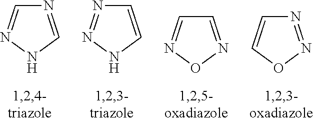

The terms “halo”, “halogen”, and “halide” are used in the conventional sense to refer to a chloro, bromo, fluoro or iodo substituent or ligand.

The term alkylene as used herein refers to an alkanediyl group which is a divalent saturated aliphatic group, with two carbon atoms as points of attachment, a linear or branched, cyclo, cyclic or acyclic structure. Exemplary alkylene includes propane-1,2-diyl group (—CH(CH3)CH2-) or propane-1,3-diyl group (—CH2CH2CH2-).

The term alkenylene refers to an alkenediyl group which is a divalent unsaturated aliphatic group, with two carbon atoms as points of attachment, a linear or branched, cyclo, cyclic or acyclic structure, at least one nonaromatic carbon-carbon double bond. Exemplary alkylene includes 2-butene-1,4-diyl group (—CH2CH═CHCH2-).

The term alkynylene refers to an alkynediyl group which is a divalent unsaturated aliphatic group, with two carbon atoms as points of attachment, a linear or branched, cyclo, cyclic or acyclic structure, at least one nonaromatic carbon-carbon triple bond. Exemplary alkylene includes 2-butyne-1,4-diyl group (—CH2C≡CCH2-).

The term “substituted” as in “substituted alkyl,” “substituted aryl,” and the like, is meant that in the alkyl, aryl, or other moiety, at least one hydrogen atom bound to a carbon (or other) atom is replaced with one or more non-hydrogen substituents.

Examples of such substituents include, without limitation: functional groups such as halo, hydroxyl, sulfhydryl, C1-C24 alkoxy, C2-C24 alkenyloxy, C2-C24 alkynyloxy, C5-C24 aryloxy, C6-C24 aralkyloxy, C6-C24 alkaryloxy, acyl (including C2-C24 alkylcarbonyl (—CO-alkyl) and C6-C24 arylcarbonyl (—CO-aryl)), acyloxy (—O-acyl, including C2-C24 alkylcarbonyloxy (—O—CO-alkyl) and C6-C24 arylcarbonyloxy (—O—CO-aryl)), C2-C24 alkoxycarbonyl (—(CO)—O-alkyl), C6-C24 aryloxycarbonyl (—(CO)—O-aryl), halocarbonyl (—CO)—X where X is halo), C2-C24 alkylcarbonato (—O—(CO)—O-alkyl), C6-C24 arylcarbonato (—O—(CO)—O-aryl), carboxy (—COOH), carboxylato (COO—), carbamoyl (—(CO)—NH2), mono-(C1-C24 alkyl)-substituted carbamoyl (—(CO)—NH(C1-C24 alkyl)), di-(C1-C24 alkyl)-substituted carbamoyl (—(CO)—N(C1-C24 alkyl)2), mono-(C5-C24 aryl)-substituted carbamoyl (—(CO)—NH-aryl), di-(C5-C24 aryl)-substituted carbamoyl (—(CO)—N(C5-C24 aryl)2), di-N—(C1-C24 alkyl), N—(C5-C24 aryl)-substituted carbamoyl, thiocarbamoyl (—(CS)—NH2), mono-(C1-C24 alkyl)-substituted thiocarbamoyl (—(CO)—NH(C1-C24 alkyl)), di-(C1-C24 alkyl)-substituted thiocarbamoyl (—(CO)—N(C1-C24 alkyl)2), mono-(C5-C24 aryl)-substituted thiocarbamoyl (—(CO)—NH-aryl), di-(C5-C24 aryl)-substituted thiocarbamoyl (—(CO)—N(C5-C24 aryl)2), di-N—(C1-C24 alkyl), N—(C5-C24 aryl)-substituted thiocarbamoyl, carbamido (—NH—(CO)—NH2), cyano(—C—N), cyanato (—O—C—N), thiocyanato (—S—C—N), formyl (—(CO)—H), thioformyl ((CS)—H), amino (—NH2), mono-(C1-C24 alkyl)-substituted amino, di-(C1-C24 alkyl)-substituted amino, mono-(C5-C24 aryl)-substituted amino, di-(C5-C24 aryl)-substituted amino, C2-C24 alkylamido (—NH—(CO)-alkyl), C6-C24 arylamido (—NH—(CO)-aryl), imino (—CR=NH where R=hydrogen, C1-C24 alkyl, C5-C24 aryl, C6-C24 alkaryl, C6-C24 aralkyl, etc.), C2-C20 alkylimino (CR=N(alkyl), where R=hydrogen, C₁-C₂₄ alkyl, C₅-C₂₄ aryl, C₆-C₂₄ alkaryl, C₆-C₂₄ aralkyl, etc.), arylimino (—CR=N(aryl), where R=hydrogen, C₁-C₂₀ alkyl, C5-C24 aryl, C6-C24 alkaryl, C6-C24 aralkyl, etc.), nitro (—NO2), nitroso (—NO), sulfo (—SO2-OH), sulfonato (—SO2-O—), C1-C24 alkylsulfanyl (—S-alkyl; also termed “alkylthio”), C5-C24 arylsulfanyl (—S-aryl; also termed “arylthio”), C1-C24 alkylsulfinyl (—(SO)-alkyl), C5-C24 arylsulfinyl (—(SO)-aryl), C1-C24 alkylsulfonyl (—SO2-alkyl), C5-C24 arylsulfonyl (—SO2-aryl), boryl (—BH2), borono (—B(OH)2), boronato (—B(OR)2 where R is alkyl or other hydrocarbyl), phosphono (—P(O)(OH)2), phosphonato (—P(O)(O⁻)2), phosphinato (—P(O)(O⁻)), phospho (—PO2), phosphino (—PH2), silyl (—SiR3 wherein R is hydrogen or hydrocarbyl), and silyloxy (—O-silyl); and the hydrocarbyl moieties C1-C24 alkyl (e.g. C1-C12 alkyl and C1-C6 alkyl), C2-C24 alkenyl (e.g. C2-C12 alkenyl and C2-C6 alkenyl), C2-C24 alkynyl (e.g. C2-C12 alkynyl and C2-C6 alkynyl), C5-C24 aryl (e.g. C5-C14 aryl), C6-C24 alkaryl (e.g. C6-C16 alkaryl), and C6-C24 aralkyl (e.g. C6-C16 aralkyl).

The term “acyl” refers to substituents having the formula —(CO)-alkyl, —(CO)-aryl, or —(CO)-aralkyl, and the term “acyloxy” refers to substituents having the formula —O(CO)— alkyl, —O(CO)-aryl, or —O(CO)-aralkyl, wherein “alkyl,” “aryl, and “aralkyl” are as defined above.

The term “alkaryl” refers to an aryl group with an alkyl substituent, and the term “aralkyl” refers to an alkyl group with an aryl substituent, wherein “aryl” and “alkyl” are as defined above. In some embodiments, alkaryl and aralkyl groups contain 6 to 24 carbon atoms, and particularly alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “Periodic Table” refers to the version of IUPAC Periodic Table of the Elements dated Nov. 28, 2016 [12].

When a Markush group or other grouping is used herein, all individual members of the group and all combinations and possible sub-combinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all subranges, as well as all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations, which is not specifically disclosed herein.

“Optional” or “optionally” means that the subsequently described circumstance may or may not occur, so that the description includes instances where the circumstance occurs and instances where it does not according to the guidance provided in the present disclosure. For example, the phrase “optionally substituted” means that a non-hydrogen substituent may or may not be present on a given atom, and, thus, the description includes structures wherein a non-hydrogen substituent is present and structures wherein a non-hydrogen substituent is not present. It will be appreciated that the phrase “optionally substituted” is used interchangeably with the phrase “substituted or unsubstituted.” Unless otherwise indicated, an optionally substituted group may have a substituent at each substitutable position of the group, and when more than one position in any given structure may be substituted with more than one substituent selected from a specified group, the substituent may be either the same or different at every position. Combinations of substituents envisioned can be identified in view of the desired features of the compound in view of the present disclosure, and in view of the features that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In summary, in several embodiments, described herein are organosilicon compound, related complex that allow performance of fluorocarbon compound or olefin-based reactions and in particular polymerization of olefins to produce polyolefin polymers, and related methods and systems are described.

In particular, it will be understood that various modifications may be made without departing from the spirit and scope of the present disclosure. Accordingly, other embodiments are within the scope of the following claims.

### REFERENCES

(1) Wuebbles, D. J.; Jain, A. K. Concerns About Climate Change and the Role of Fossil Fuel Use. Fuel Process. Technol. 2001, 71 (1-3), 99-119. Accessed: Apr. 23, 2024.
(2) Boden, T. A.; Marland, G.; Andres, R. J. Global, Regional, and National Fossil-Fuel CO2 Emissions. Carbon Dioxide Inf Anal. Cent. Oak Ridge Natl. Lab. USA Oak Ridge TN Dep. Energy 2009. Accessed: Apr. 23, 2024.
(3) Hack, J.; Maeda, N.; Meier, D. M. Review on CO2 Capture Using Amine-Functionalized Materials. ACS Omega 2022, 7 (44), 39520-39530. Accessed: May 3, 2024.
(4) Diemert, K.; Hahn, T.; Kuchen, W. Zur Kenntnis Des Natriumdiphenylphosphinoformiats Ph2PCOONa. Phosphorus, Sulfur Silicon Relat. Elem. 1991, 60 (3-4), 287-294. doi.org/10.1080/10426509108036793. Accessed: May 3, 2024.
(5) Musgrave, C. B.; Prokofjevs, A.; Goddard, W. A. Phosphine Modulation for Enhanced CO2 Capture: Quantum Mechanics Predictions of New Materials. J. Phys. Chem. Lett. 2022, 13 (48), 11183-11190. Accessed: Jul. 20, 2024.
(6) Buβ, F.; Mehlmann, P.; Miick-Lichtenfeld, C.; Bergander, K.; Dielmann, F. Reversible Carbon Dioxide Binding by Simple Lewis Base Adducts with Electron-Rich Phosphines. J. Am. Chem. Soc. 2016, 138 (6), 1840-1843. Accessed: Apr. 12, 2024.
(7) Xu, Y. F.; Duchesne, P. N.; Wang, L.; et al. High-Performance Light-Driven Heterogeneous CO2 Catalysis with Near-Unity Selectivity on Metal Phosphides. Nat. Commun. 2020, 11, 5149. Accessed: Jul. 18, 2024.
(8) Welch, G. C.; San Juan, R. R.; Masuda, J. D.; Stephan, D. W. Reversible, Metal-Free Hydrogen Activation. Science 2006, 314, 1124-1126. https://doi.org/10.1126/science.1134230. Accessed: May 23, 2024.
(9) Harhausen, M.; Fröhlich, R.; Kehr, G.; Erker, G. Reactions of Modified Intermolecular Frustrated P/B Lewis Pairs with Dihydrogen, Ethene, and Carbon Dioxide. Organometallics 2012, 31 (7), 2801-2809. doi.org/10.1021/om201076f. Accessed: Apr. 23, 2024.
(10) Dornhaus, F.; Bolte, M.; Lerner, H.-W.; Wagner, M. Phosphanylborohydrides: First Assessment of the Relative Lewis Basicities of [BH3PPh2]—, CH3PPh2, and HPPh2. Eur. J. Inorg. Chem. 2006, 2006, 1777-1785. doi.org/10.1002/ejic.200501126. Accessed: Apr. 28, 2024.
(11) Lebel, H.; Morin, S.; Paquet, V. Alkylation of Phosphine Boranes by Phase-Transfer Catalysis. Org. Lett. 2003, 5 (13), 2347-2349. doi.org/10.1021/ol0347139. Accessed: Jul. 12, 2024.
(12) Barozzino Consiglio, G.; Queval, P.; Harrison-Marchand, A.; Mordini, A.; Lohier, J.-F.; Delacroix, O.; Gaumont, A.-C.; Gérard, H.; Maddaluno, J.; Oulyadi, H. Ph₂P(BH3)Li: From Ditopicity to Dual Reactivity. J. Am. Chem. Soc. 2011, 133 (16), 6472-6480. https://doi.org/10.1021/ja201760c. Accessed: Jul. 19, 2024.
(13) Lebel, H.; Morin, S.; Paquet, V. Alkylation of Phosphine Boranes by Phase-Transfer Catalysis. Org. Lett. 2003, 5 (13), 2347-2349. doi.org/10.1021/ol0347139. Accessed: Jul. 21, 2024.
(14) Valentin, M.; Montes, E.; Adam, W. Counterion Effects in the Nucleophilic Substitution Reaction of the Acetate Ion with Alkyl Bromides in the Synthesis of Esters. J. Chem. Educ. 2009, 86 (11). Accessed: Jul. 12, 2024.
(15) Buβ, F.; Röthel, M. B.; Werra, J. A.; Rotering, P.; Wilm, L. F. B.; Daniliuc, C. G.; Löwe, P.; Dielmann, F. Tris(tetramethylguanidinyl)phosphine: The Simplest Non-ionic Phosphorus Superbase and Strongly Donating Phosphine Ligand. Chem. Eur. J. 2022, 28, e202104021. Accessed: Jul. 14, 2024.
(16) Buβ, F.; Mehlmann, P.; Miick-Lichtenfeld, C.; Bergander, K.; Dielmann, F. Reversible Carbon Dioxide Binding by Simple Lewis Base Adducts with Electron-Rich Phosphines. J. Am. Chem. Soc. 2016, 138 (6), 1840-1843. https://doi.org/10.1021/jacs.5b13116. Accessed: Jul. 11, 2024.
(17) Mehlmann, P.; Muck-Lichtenfeld, C.; Tan, T. T.; Dielmann, F. Tris (imidazolin-2-ylidenamino) phosphine: A Crystalline Phosphorus (III) Superbase That Splits Carbon Dioxide. Chem. Eur. J. 2017, 23 (25), 5929-5933. Accessed: Jul. 16, 2024.
(18) Diemert, K.; Hahn, T.; Kuchen, W.; Tommes, P. Darstellung und Reaktionen Einiger Lithiumphosphinoformiate R2PCOOLi und RHPCOOLi—Ab Initio-Rechnungen im System H2PCOOH—PH3/CO₂. Phosphorus, Sulfur Silicon Relat. Elem. 1993, 83 (1-4), 65-76. Accessed: Jul. 20, 2024.
(19) Stankevič, M.; Pietrusiewicz, K. M. An Expedient Reduction of sec—Phosphine Oxides to sec—Phosphine-Boranes by BH3·SMe2. Synlett 2003, 2003 (7), 1012-1016. Accessed: Apr. 8, 2024.
(20) Jaska, C. A.; Lough, A. J.; Manners, I. Linear Hybrid Aminoborane/Phosphinoborane Chains: Synthesis, Proton-Hydride Interactions, and Thermolysis Behavior. Inorg. Chem. 2004, 43 (3), 1090-1099. Accessed: Apr. 9, 2024.
(21) Zhao, Y.; Truhlar, D. G. Density Functionals with Broad Applicability in Chemistry. Acc. Chem. Res. 2008, 41 (2), 157-167. Accessed: Mar. 25, 2024.
(22) Grimme, S.; Antony, J.; Ehrlich, S.; Krieg, H. A Consistent and Accurate Ab Initio Parametrization of Density Functional Dispersion Correction (DFT-D) for the 94 Elements H—Pu. J. Chem. Phys. 2010, 13 (15). Accessed: Mar. 25, 2024.
(23) Hehre, W. J.; Ditchfield, K.; Pople, J. A. Self-Consistent Molecular Orbital Methods. XII. Further Extensions of Gaussian-Type Basis Sets for Use in Molecular Orbital Studies of Organic Molecules. J. Chem. Phys. 1972, 56 (5), 2257-2261. Accessed: Mar. 25, 2024.
(24) Takano, Y.; Houk, K. N. Benchmarking the Conductor-like Polarizable Continuum Model (CPCM) for Aqueous Solvation Free Energies of Neutral and Ionic Organic Molecules. J. Chem. Theory Comput. 2004, 1 (1), 70-77. Accessed: Mar. 25, 2024.
ADDIN EN.REFLIST

1. A method for capturing carbon dioxide from a target environment, the method comprising:
contacting the target environment with at least one phosphorous boron-bonded compound
the contacting performed for a time and under condition to allow binding of CO₂ if present with the at least one phosphorous boron-bonded compound to form a phosphino-borane-carbon dioxide complex
wherein the at least one phosphorous boron-bonded compound comprises one or more of
(i) a phosphine-borane of Formula I 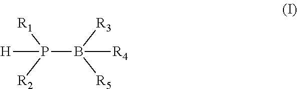
in which R₁, R₂, R₃, R₄ and R₅ are each independently H, F or a substituted or unsubstituted alkyl, aryl, alkoxy, aryloxy or dialkylamino group, or group of Formula II 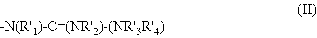
in which R′₁, R′₂, R′₃, and R′₄ are independently a substituted or unsubstituted alkyl or aryl group, and
in which R₁, R₂, R₃, R₄ and R₅ are configured to provide a P—H bond having pKa 5≤pKa≤25 at 298K, at 1 atm
(ii) a cyclic phosphine-borane of Formula III 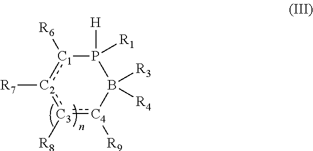
in which
n is 0 or 1
R₁, R₃, and R₄ are each independently H, F or a substituted or unsubstituted alkyl, aryl, alkoxy, aryloxy or dialkylamino group, or group of Formula II 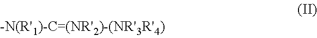
in which R′₁, R′₂, R′₃, and R′₄ are independently a substituted or unsubstituted alkyl or aryl group
R₆ is H or is joined to R₇ to form a substituted or unsubstituted and optionally heteroatom containing aliphatic or aromatic 5 membered or 6-membered ring
R₇ is H or is joined to R₆ or R₈ to form a substituted or unsubstituted and optionally heteroatom containing aliphatic or aromatic 5 membered or 6-membered ring
R₈ is H or is joined to R₇ or R₉ to form a substituted or unsubstituted and optionally heteroatom containing aliphatic or aromatic 5 membered or 6-membered ring and
R₉ is H or is joined to R₈ to form a substituted or unsubstituted and optionally heteroatom containing aliphatic or aromatic 5 membered or 6-membered ring
and
(iii) a phosphine-borane salt of Formula IV 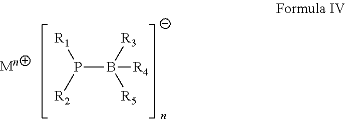
in which n is an integer from 1 to 3
R₁, R₂, R₃, R₄ and R₅ are each independently H, F or a substituted or unsubstituted alkyl, aryl, alkoxy, aryloxy or dialkylamino group, or group of Formula II 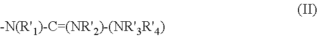
in which R′₁, R′₂, R′₃, and R′₄ are independently a substituted or unsubstituted alkyl or aryl group,
M is Li, Na, K, Rb, Cs or tetraalkylammonium when n=1; Mg, Ca, Sr, Ba when n=2 or Al when n=3
and in which R₁, R₂, R₃, R₄ and R₅ are in configured such that the phosphino-borane exhibits a reactivity toward CO₂ with a ΔG value <0 kcal/mol at 298K, at 1 atm,
wherein the phosphino-borane-carbon dioxide complex is a complex of Formula V 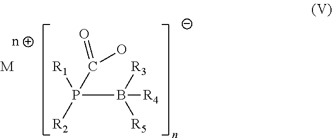
in which
n is an integer from 1 to 3
R₁, R₂, R₃, R₄ and R₅ are each independently H, F or a substituted or unsubstituted alkyl, aryl, alkoxy, aryloxy or dialkylamino group, or group of Formula II 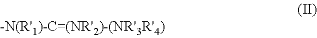
in which R′₁, R′₂, R′₃, and R′₄ are independently a substituted or unsubstituted alkyl or aryl group, and
M is Li, Na, K, Rb, Cs or tetraalkylammonium when n=1; Mg, Ca, Sr, Ba when n=2 or Al when n=3,
and wherein when the at least one phosphorous boron-bonded compound comprises a phosphino-borane of Formula (I) and/or a cyclic phosphino-borane of Formula (III), the contacting is performed in presence of a base.

2. The method of claim 1, wherein the method is directed to an irreversible CO₂, and wherein in the phosphine-borane of Formula I, the cyclic phosphine-borane of Formula III and the phosphine-borane salt of Formula IV, Mnⁿ⁺=Li, Na, Mg, Al and substituents are electro donating groups.

3. The method of claim 2, wherein R₁ and R₂ are electro donating groups.

4. The method of claim 3, wherein the electron donating group are alkyl groups.

5. The method of claim 1, wherein the method is directed to a reversible CO₂ capture, and wherein the phosphine-borane of Formula I, the cyclic phosphine-borane of Formula III and the phosphine-borane salt of Formula IV, M⁺=K, Rb, Cs, tetraalkylammonium and substituents are electro withdrawing groups.

6. The method of claim 1, wherein R₁ and R₂ are electro withdrawing groups.

7. The method of claim 6, wherein the electron donating group are aryl groups.

8. The method of claim 2, wherein M=Li, Na, K in Formula V.

9. The method of claim 1, wherein the contacting is performed by exposing the phosphino-borane complex to a gaseous stream from the target environment.

10. The method of claim 1, wherein the base is an alkoxide, hydroxide, amide, aryloxide, amine or silazide.

11. The method of claim 1, wherein the at least one phosphorous boron-bonded compound comprises a compound of Formula I and/or Formula III and the method further comprises contacting the at least one phosphorous boron-bonded compound base is between 10 seconds and 1 hour.

12. The method of claim 1, wherein the method further comprises mixing the least phosphorous boron-bonded compound with a vehicle to provide a phosphorous boron-bonded composition.

13. The method of claim 12, wherein the vehicle comprises a solid sorbent having a surface configured for contacting a target environment and the contacting is performed on the surface of the solid sorbent presenting the phosphino-borane absorbed on the surface.

14. The method of claim 13, where the solid sorbent comprises a sorbent material that is porous, with pore sizes ranges between 1-500 nm.

15. The method of claim 13, where the sorbent material has a surface area from 0.1-100 m²/g.

16. The method of claim 13, where the solid sorbent is a polymer material, a silica or alumina material, a zeolite or a metal-organic framework.

17. A method for converting carbon dioxide from a target environment to a carbon dioxide reaction product, the method comprising:
capturing the carbon dioxide from the target environment by performing the method of claim 8, to provide the phosphorous boron-bonded compound of Formula V,
reacting the compound of Formula V with a reducing agent and/or an alkylating agent to obtain the carbon dioxide reaction product.

18. The method of claim 17, wherein the phosphorous boron-bonded compound of Formula V is within a composition further comprising a vehicle and reacting is performed within the composition.

19. The method of claim 17 wherein the reducing agent is hydrogen gas, hydrogen chloride or a metal hydride or borohydride reducing agent.

20. The method of claim 17, wherein the reducing agent is an electrode configured to donate electrons to a solution so as to reduce a compound present therein, thereby functioning as a reducing agent during the reacting, the reacting being an electrochemical reduction.

21. The method of claim 17, where the alkylating agent is an alkyl halide, an alkyl triflate or an alkyl sulfonate.

22. The method of claim 21, wherein the reacting with and alkylating agent is performed to obtain a carbonate ester or a polycarbonate.

23. A method for making carbon dioxide reaction products, the method comprising:
reacting the compound of Formula V with a reducing agent and/or an alkylating agent to obtain the carbon dioxide reaction product of Formula VI 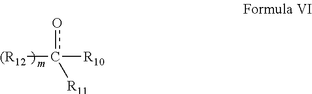
wherein
m is 0 or 1;
R₁₀, =H, OH, O-alkyl, O-aryl, CH₃, C(OH)H₂, C(O)H or C(O)OH
R₁₁ and R₁₂ are each independently H, OH, O-alkyl or O-aryl and
wherein when m=0 and R₁₀=OH, R₁₁ is H, O-alkyl or O-aryl.

24. The method of claim 23 where the alkylating agent is an alkyl halide, alkyl triflate or alkyl sulfonate, and m=0, R₁₀=O-alkyl and R₁₁=O-alkyl.

25. The method of claim 23 where the alkylating agent is an alkyl halide, alkyl triflate or alkyl sulfonate, and m=0, R₁₀=O-alkyl and R₁₁=O-alkyl such that the product of Formula VI is a polymer with the polymer chain joined through R₁₀ and R₁₁.

26. The method of claim 23 where the product of Formula VI is formic acid.

27. The method of claim 23 where the product of Formula VI is methanol.

28. The method of claim 23 where the product of Formula VI is acetic acid.

29. The method of claim 23 where the product of Formula VI is ethanol.

30. The method of claim 23 wherein a further reduction of the initial product of Formula VI occurs to provide methane as the final product.
